SEQUENCE LISTING (34 sequences)

SEQ ID NO: 1 - DNA, 645 bp, Mus musculus
atcctgaagggtataaaatgaacggagagtgggtcatcacagcagtaactcactcatgcaaagtaagatcctgcagtagtgggtggagaagagtgagcgcatacgagtggagagaaaaatattctgcttcagtgaagagctcgggttgttcaaagcatgagtgctgccgtcctcctctccgtgctttttgctcttgcttgtgggcaagcagcatccttttgtattcccactgagtatacaatgtacgtggataggagagagtgtgcctactgcctgaccatcaacaccaccatctgtgctgggtattgtatgacacgggatatcaatggcaaactgtttcttcccaaatatgcactctctcaggatgtctgtacatacagagacttcatctacagaacggtggaaataccaggatgcccgcaccatgttactccttatttctccttccctgtcgccgtaagctgcaagtgtggcaagtgtaatactgacaacagtgactgcatacacgaggctgtcagaaccaactactgcaccaagccgcagtctttctatctggggggattttctgtttaacttcaatagcagtgcaatctggttaaatgtgtttacctggaatagaacgaataaaataccattgagacgtct

SEQ ID NO: 2 - protein, 138 aa, Mus musculus
MSAAVLLSVLFALACGQAASFCIPTEYTMYVDRRECAYCLTINTTICAGYCMTRDINGKLFLPKYALSQDVCTYRDFIYRTVEIPGCPHHVTPYFSFPVAVSCKCGKCNTDNSDCIHEAVRTNYCTKPQSFYLGGFSV

SEQ ID NO: 3 - DNA, 354 bp, Mus musculus
atgttaagatctcttttctttccacaggatatcaatggcaaactgtttcttcccaaatatgcactctctcaggatgtctgtacatacagagacttcatctacagaacggtggaaataccaggatgcccgcaccatgttactccttatttctccttccctgtcgccataagctgcaagtgtggcaagtgtaatactgacaacagtgactgcatacacgaggctgtcagaaccaactactgcaccaagccgcagtctttctatctggggggattttctgtttaacttcaatagcagtgcaatctggttaaatgtgtttacctggaatagaacgaataaaataccattgagacgtct

SEQ ID NO: 4 - protein, 93 aa, Mus musculus
MLRSLFFPQDINGKLFLPKYALSQDVCTYRDFIYRTVEIPGCPHHVTPYFSFPVAVSCKCGKCNTDNSDCIHEAVRTNYCTKPQSFYLGGFSV

SEQ ID NO: 5 - DNA, 578 bp, Homo sapiens
ggtataaaatgaaccaagagcttttagtttgggtcaccacagcatctgctcaccaatgcaaagtaagcatgactgctctctttctgatgtccatgctttttggccttacatgtgggcaagcgatgtctttttgtattccaactgagtatacaatgcacatcgaaaggagagagtgtgcttattgcctaaccatcaacaccaccatctgtgctggatattgtatgacacgggatatcaatggcaaactgtttcttcccaaatatgctctgtcccaggatgtttgcacatatagagacttcatctacaggactgtagaaataccaggatgcccactccatgttgctccctatttttcctatcctgttgctttaagctgtaagtgtggcaagtgcaatactgactatagtgactgcatacatgaagccatcaagacaaactactgtaccaaacctcagaagtcttatctggtaggattttctgtctaatagtgatataatttgcaatttggttaaatgtgcttgcctgaaataaagctaataaaaatattatgtttcacattatcttctgttcattttgag

SEQ ID NO: 6 - protein, 138 aa, Homo sapiens
MTALFLMSMLFGLTCGQAMSFCIPTEYTMHIERRECAYCLTINTTICAGYCMTRDINGKLFLPKYALSQDVCTYRDFIYRTVEIPGCPLHVAPYFSYPVALSCKCGKCNTDYSDCIHEAIKTNYCTKPQKSYLVGFSV

SEQ ID NO: 7 - DNA, 375 bp, Homo sapiens
atgctctcttttctgttctttccccaggatatcaatggcaaactgtttcttcccaaatatgctctgtcccaggatgtttgcacatatagagacttcatctacaggactgtagaaataccaggatgcccactccatgttgctccctatttttcctatcctgttgctttaagctgtaagtgtggcaagtgcaatactgactatagtgactgcatacatgaagccatcaagacaaactactgtaccaaacctcagaagtcttatctggtaggattttctgtctaatagtgatataatttgcaatttggttaaatgtgcttgcctgaaataaagctaataaaaatattatgtttcacattatcttctgttcattttgag

SEQ ID NO: 8 - protein, 93 aa, Homo sapiens
MLSFLFFPQDINGKLFLPKYALSQDVCTYRDFIYRTVEIPGCPLHVAPYFSYPVALSCKCGKCNTDYSDCIHEAIKTNYCTKPQKSYLVGFSV

SEQ ID NO: 9 - DNA, 374 bp, Mus musculus
cgactggagcacgaggacactgaccagagctcaggagtcctttattgatcatcactgatacaaagggatatcagtaagttgtggtctttctattacactatctaaagttgtgtggagtttatcatgttaagatctcttttctttccacaggatatcaatggcaaactgtttcttcccaaatatgcactctctcaggatgtctgtacatacagagacttcatctacagaacggtggaaataccaggatgcccgcaccatgttactccttatttctccttccctgtcgccataagctgcaagtgtggcaagtgtaatactgacaacagtgactgcatacacgaggctgtcagaaccaactactgcaccaagccgca

SEQ ID NO: 10 - DNA, 312 bp, Homo sapiens
attatgctctcttttctgttctttccccaggatatcaatggcaaactgtttcttcccaaatatgctctgtcccaggatgtttgcacatatagagacttcatctacaggactgtagaaataccaggatgcccactccatgttgctccctatttttcctatcctgttgctttaagctgtaagtgtggcaagtgcaatactgactatagtgactgcatacatgaagccatcaagacaaactactgtaccaaacctcagaagtcttatctggtaggattttctgtctaatagtgatataatttgcaatttggttaa

SEQ ID NO: 11 - DNA, 55 bp, Artificial Sequence
  Synthetic PCR Primer
gatccatctcttttctttccacagttcaagagactgtggaaagaaaagagatcta

SEQ ID NO: 12 - DNA, 55 bp, Artificial Sequence
  Synthetic PCR Primer
agcttagatctcttttctttccacagtctcttgaactgtggaaagaaaagagatg

SEQ ID NO: 13 - DNA, 20 bp, Artificial Sequence
  Synthetic PCR Primer
aagagctcgggttgttcaaa

SEQ ID NO: 14 - DNA, 22 bp, Artificial Sequence
  Synthetic PCR Primer
cacatttaaccagattgcactg

SEQ ID NO: 15 - DNA, 20 bp, Artificial Sequence
  Synthetic PCR Primer
tctccgtgctttttgctctt

SEQ ID NO: 16 - DNA, 20 bp, Artificial Sequence
  Synthetic PCR Primer
gcaagcagcatccttttgta

SEQ ID NO: 17 - DNA, 21 bp, Artificial Sequence
  Synthetic PCR Primer
cgtggataggagagagtgtgc

SEQ ID NO: 18 - DNA, 20 bp, Artificial Sequence
  Synthetic PCR Primer
tcaacaccaccatctgtgct

SEQ ID NO: 19 - DNA, 21 bp, Artificial Sequence
  Synthetic PCR Primer
tgctgggtattgtatgacacg

SEQ ID NO: 20 - DNA, 20 bp, Artificial Sequence
  Synthetic PCR Primer
ccgcaccatgttactcctta

SEQ ID NO: 21 - DNA, 20 bp, Artificial Sequence
  Synthetic PCR Primer
acagcctcgtgtatgcagtc

SEQ ID NO: 22 - DNA, 24 bp, Artificial Sequence
  Synthetic PCR Primer
cgactggagcacgaggacactgac

SEQ ID NO: 23 - DNA, 27 bp, Artificial Sequence
  Synthetic PCR Primer
tgcggcttggtgcagtagttggttctg

SEQ ID NO: 24 - DNA, 24 bp, Artificial Sequence
  Synthetic PCR Primer
atcatgttaagatctcttttcttt

SEQ ID NO: 25 - DNA, 23 bp, Artificial Sequence
  Synthetic PCR Primer
aaccagattgcactgctattgaa

SEQ ID NO: 26 - DNA, 23 bp, Artificial Sequence
  Synthetic PCR Primer
ttgttcaatgcatttcttttagc

SEQ ID NO: 27 - DNA, 22 bp, Artificial Sequence
  Synthetic PCR Primer
gaaaggaagtggggataaatca

SEQ ID NO: 28 - DNA, 24 bp, Artificial Sequence
  Synthetic PCR Primer
gatgggttaattgtagatgtgtgg

SEQ ID NO: 29 - DNA, 23 bp, Artificial Sequence
  Synthetic PCR Primer
cagagctcaggagtcctttattg

SEQ ID NO: 30 - DNA, 20 bp, Artificial Sequence
  Synthetic PCR Primer
agcatgactgctctctttct

SEQ ID NO: 31 - DNA, 25 bp, Artificial Sequence
  Synthetic PCR Primer
aaccaaattgcaaattatatcacta

SEQ ID NO: 32 - DNA, 24 bp, Artificial Sequence
  Synthetic PCR Primer
attatgctctcttttctgttcttt

SEQ ID NO: 33 - DNA, 23 bp, Artificial Sequence
  Synthetic PCR Primer
atggattactacagaaaatatgc

SEQ ID NO: 34 - DNA, 24 bp, Artificial Sequence
  Synthetic PCR Primer
agatttgtgataataacaagtact

(19) United States
(12) Patent Application Publication
(10) Pub. No.: US 2011/0052591 A1
(43) Pub. Date: Mar. 3, 2011

(54) Variants of Thyroid Stimulating Hormone Beta

(75) Inventors: JOHN R. KLEIN, HOUSTON, TX (US); JEREMY S. SCHAEFER, PEARLAND, TX (US); BRYCE H. VINCENT, HOUSTON, TX (US)

(73) Assignee: BOARD OF REGENTS OF THE UNIVERSITY OF TEXAS SYSTEM, AUSTIN, TX (US)

(21) Appl. No.: 12/871,716

(22) Filed: Aug. 30, 2010

Related U.S. Application Data
(60) Provisional application No. 61/238,078, filed on Aug. 28, 2009.

(51) Int. Cl. C07K 14/575; C12N 15/16; C12N 15/85; C12N 15/86; C12N 5/10; C12P 21/02; C07K 16/26; A61K 39/395

(52) U.S. Cl. 424/139.1; 536/23.1; 435/320.1; 435/325; 435/365; 435/358; 435/364; 435/367; 435/369; 435/354; 435/252.33; 435/69.4; 530/324; 530/387.9; 514/9.7

(57) ABSTRACT

Polynucleotide and polypeptide sequences that encode novel variants of mouse or human thyroid stimulating hormone-β proteins are disclosed that can be used in therapeutic, diagnostic, and pharmacogenomic applications to prevent, treat or reduce the severity of thyroid stimulating hormone-β-related disorders.

### CROSS REFERENCE TO RELATED APPLICATIONS

This application claims priority to U.S. Provisional Patent Application No. 61/238,078, filed on Aug. 28, 2009, the entirety of which is incorporated herein by reference.

### STATEMENT REGARDING FEDERALLY SPONSORED RESEARCH OR DEVELOPMENT

This invention was made with U.S. Government support under Grant Nos. DK035566 and DE015355 awarded by the National Institutes of Health. The government has certain rights in the invention.

### FIELD OF THE INVENTION

The present invention generally relates to the fields of endocrinology and immunology. More particularly, but not by way of limitation, the present invention pertains to the discovery and characterization of previously unrecognized polynucleotides and amino acids encoding proteins that are expressed splice variants of the human and mouse TSH-beta (TSHβ) gene (aliases include thyroid stimulating hormone beta-subunit, thyrotropin beta-subunit, TSHβ, TSHB, TSHB-beta, TSHBB CHNG4, TSH-BETA, OTTHUMP00000013654; and thyrotropin beta subunit). The present invention also encompasses the described polynucleotides, host cell expression systems, the encoded proteins, fusion proteins, polypeptides and peptides, antibodies to the encoded proteins and peptides, and genetically engineered animals that either lack or overexpress the disclosed polynucleotides, antagonists and agonists of the proteins, and other compounds that modulate the expression or activity of the proteins encoded by the disclosed polynucleotides.

### BACKGROUND OF THE INVENTION

Thyroid-stimulating hormone (TSH: also known as TSHB or thyrotropin) is a peptide hormone synthesized and secreted by thyrotrope cells in the anterior pituitary gland which regulates the endocrine function of the thyroid gland. The TSH receptor is found mainly on thyroid follicular cells where stimulation of the receptor by TSH increases triiodothyronine (T3) and thyroxine (T4) production and secretion. Thus, TSH stimulates the rate of release of T3 and T4, which influence many aspects of mammalian physiology, including basal metabolism, growth, development, mood, and cognition. TSH induces thyroid hormone synthesis by promoting the proteolytic conversion of thyroglobulin to T4 and T3 in thyroid follicles. Levels of thyroid hormones are controlled by the presence of circulating pituitary-derived TSH. In addition, feedback mechanisms, such as those responsive to T4 levels, modulate TSH synthesis via hypothalamic-derived thyrotropin releasing hormone (TRH).

TRH is manufactured in the hypothalamus and transported to the anterior pituitary gland via the superior hypophyseal artery, where it increases TSH production and release. Somatostatin is also produced by the hypothalamus, and has an opposite effect on the pituitary production of TSH by decreasing or inhibiting its release. The level of thyroid hormones (e.g, T3 and T4) in the blood also has an effect on the pituitary release of TSH. For example, when the levels of T3 and T4 are low, the production of TSH is increased. Conversely, when levels of T3 and T4 are high, the TSH production is decreased. This effect creates a regulatory negative feedback loop. The production of antibodies that bind the TSHB receptor can mimic TSH action and such antibodies are found in patients with Graves' disease.

TSH, luteinizing hormone (LH), follicle-stimulating hormone (FSH), and chorionic gonadotropin (HCG: somatostatin) are members of a family of glycoprotein hormones that share a common α-subunit and have unique β-subunits. It is the β-subunit, therefore, that is responsible for hormone specificity.

Irregular TSHβ levels are associated with numerous medical conditions and diseases (i.e., TSHβ-related disorders). Such TSHβ-related disorders include adenoma, thyroid hormone resistance, hypopituitarism, hyperthyroidism, Graves' disease, congenital hypothyroidism (cretinism), hypothyroidism, and Hashimoto's thyroiditis. Current methods to treat TSHβ-related disorders have limitations. Thus, there is currently a need for polynucleotides, host cell expression systems, proteins, polypeptides, peptides, antibodies, genetically engineered animals, antagonists, agonists, and other compounds that can be used for the diagnosis, drug screening, clinical trial monitoring, and treatment of TSHβ-related disorders.

### BRIEF SUMMARY OF THE INVENTION

In some embodiments, the disclosure describes the discovery, identification, and characterization of nucleotides that encode novel variants of both mouse and human TSHβ (e.g., isolated nucleic acid molecules of SEQ ID NOS: 3 or 7). The disclosure also describes the corresponding amino acid sequences of these nucleotides (e.g., the amino acid sequence of SEQ ID NOS: 4 or 8).

In further embodiments, the disclosure pertains to expression vectors that comprise the isolated nucleic acid molecules of SEQ ID NOS: 3 or 7. In some embodiments, the expression vectors may be plasmids, cosmids, bacteriophages, and/or viral expression vectors (e.g., baculoviruses, cauliflower mosaic viruses, CaMV, tobacco mosaic viruses, and TMV). Further embodiments of the present disclosure pertain to host cells that comprise the above-mentioned expression vectors. In various embodiments, such host cells can be eukaryotic cells or prokaryotic cells.

In further embodiments, the above-described host cells may be used to produce polypeptides that comprise the amino acid sequence of SEQ ID NOS: 4 or 8. Such embodiments can utilize methods that comprise culturing host cells under conditions that permit the expression of the expression vector in the host cell.

Other embodiments of the present disclosure pertain to substantially isolated polypeptides that comprise the amino acid sequence of SEQ ID NOS: 4 or 8. Further embodiments of the present disclosure pertain to a substantially isolated antibody that binds TSH or TSH-β, wherein the antibody has immunospecificity for an epitope that comprises the first 9 amino acids of SEQ ID NOS: 4 or 8.

In additional embodiments, the present disclosure pertains to methods of treating a subject with a TSH-β-related disorder. In some embodiments, the method comprises the administration of a TSH protein to the subject (e.g., a TSH protein comprising a variant TSHβ chain that comprises the amino acid sequence of SEQ ID NOS: 4 or 8). In some embodiments, the method comprises the administration of an antagonist to the subject (e.g., an antagonist that has specificity for an epitope comprising the first 9 amino acids of SEQ ID NOS: 4 or 8).

The disclosure also describes agonists and antagonists of the described TSHβ proteins, including small molecules, large molecules, mutant variant TSHβ proteins, or portions thereof, that compete with native variant TSHβ proteins, peptides, and antibodies, as well as nucleotide sequences that can be used to inhibit the expression of the described variant TSHβ proteins (e.g., antisense and ribozyme molecules, and open reading frame or regulatory sequence replacement constructs) or to enhance the expression of the described variant TSHβ proteins (e.g., expression constructs that place the described polynucleotide under the control of a strong promoter system), and transgenic animals that express a transporter protein sequence, or “knock-outs” (which can be conditional) that do not express a functional transporter protein.

The unique TSHβ encoding sequences described in SEQ ID NOS: 1-8 are useful for, among other things, to diagnose and screen for thyroid disorders, to screen newborns for an underactive thyroid, monitor thyroid replacement therapy in people with hypothyroidism, to diagnose and monitor infertility problems, and to treat or diagnose TSHβ-related disorders.

These unique TSHβ encoding sequences are also useful for the identification of protein coding sequences, and mapping a unique gene to a particular chromosome. These sequences identify biologically verified exon splice junctions, as opposed to splice junctions that may have been bioinformatically predicted from genomic sequence alone. The sequences of the present invention are also useful as additional DNA markers for restriction fragment length polymorphism (RFLP) analysis, in population biology and in forensic biology, particularly given the presence of nucleotide polymorphisms within the described sequences.

Processes are also described for identifying compounds that modulate, i.e., act as agonists or antagonists of, TSHβ protein expression and/or TSHβ protein activity that utilize purified preparations of the described variant TSHβ proteins and/or TSH protein gene products, or cells expressing the same.

The above-described polynucleotides, host cell expression systems, proteins, polypeptides, peptides, antibodies, genetically engineered animals, antagonists, agonists, and other compounds can be used for the diagnosis, drug screening, clinical trial monitoring, and the treatment of TSHβ-related disorders. Such compounds can also be used as therapeutic agents for the treatment of any of a wide variety of symptoms associated with TSHβ-related disorders.

### BRIEF DESCRIPTION OF THE SEQUENCE LISTING

The Sequence Listing provides the known sequence of mouse and human TSH and novel variants thereof. These mouse and human TSHβ polynucleotide sequences (SEQ ID NOS: 1, 3, 5 and 7) and encoded amino acid sequences (SEQ ID NOS: 2, 4, 6 and 8) of the described proteins are presented in the Sequence Listing. PCR-amplified nucleotides encoding mouse and human TSHβ are also provided (SEQ ID NOS: 9 and 10). Also provided in the Sequence Listing are shRNA oligos used for RNA-inhibition of TSHβ splice variants (SEQ ID NOS: 11 and 12). Various synthetic primers used for PCR amplification of mouse and human TSHβ nucleotides are also provided in the Sequence Listing (SEQ ID NOS: 13-34). The contents of the Sequence Listing are also summarized in the table below:

| SEQ ID NO. | Description |
|---|---|
| 1 | Mouse Full Length Native TSHβ DNA |
| 2 | Mouse Full Length Native TSHβ Protein |
| 3 | Mouse Variant TSHβ DNA |
| 4 | Mouse Variant TSHβ Protein |
| 5 | Human Full Length Native TSHβ DNA |
| 6 | Human Full Length Native TSHβ Protein |
| 7 | Human Variant TSHβ DNA |
| 8 | Human Variant TSHβ Protein |
| 9 | 5′ RACE Product of Mouse TSHβ. see FIG. 1C |
| 10 | PCR-amplified Product of Novel Human TSHβ. See FIG. 8 |
| 11 | FWD shRNA Oligo. See FIG. 12B |
| 12 | RVR shRNA Oligo. See FIG. 12B |
| 13 | 470 FWD Mouse PCR Primer |
| 14 | 470 RVR Mouse PCR Primer |
| 15 | UP1 FWD Mouse PCR Primer |
| 16 | UP2 FWD Mouse PCR Primer |
| 17 | UP3 FWD Mouse PCR Primer |
| 18 | UP4 FWD Mouse PCR Primer |
| 19 | UP5 FWD Mouse PCR Primer |
| 20 | 98 FWD Mouse PCR Primer |
| 21 | 98 RVR Mouse PCR Primer |
| 22 | 5′ RACE Oligo FWD Mouse PCR Primer |
| 23 | TSHβ GSP RVR Mouse PCR Primer |
| 24 | Novel TSHβ FWD Mouse PCR Primer |
| 25 | Novel TSHβ RVR Mouse PCR Primer |
| 26 | Intron Primer 4 (FWD Mouse PCR Primer) |
| 27 | Intron Primer 3 (FWD Mouse PCR Primer) |
| 28 | Intron Primer 2 (FWD Mouse PCR Primer) |
| 29 | Intron Primer 1 (FWD Mouse PCR Primer) |
| 30 | FWD TSHβ Human Native PCR Primer |
| 31 | RVR TSHβ Human Native and Novel PCR Primer |
| 32 | FWD TSHβ Human Novel PCR Primer |
| 33 | FWD TSHα Human PCR Primer |
| 34 | RVR TSHα Human PCR Primer |

### BRIEF DESCRIPTION OF THE FIGURES

In order that the manner in which the above recited and other advantages and objects of the invention are obtained, a more particular description of the invention briefly described above will be rendered by reference to specific embodiments thereof, which are illustrated in the appended Figures. Understanding that these Figures depict only typical embodiments of the invention and are therefore not to be considered limiting of its scope, the invention will be described with additional specificity and detail through the use of the accompanying Figures in which:

FIG. 1 illustrates the characterization of the mouse novel TSHβ splice variant produced in bone marrow (BM) cells.

FIG. 1A (SEQ ID NO. 1), is the full-length mouse TSHβ mRNA sequence showing the locations of the five TSHβ exons (E1-E5), and the primers used for qRT-PCR (SEQ ID NOS: 13-21). The TSHβ transcript is produced starting with the second nucleotide of E4, which is the beginning of the ATG methionine codon, and extends to the TAA stop codon at the distal end of E5.

FIG. 1B shows the results of qRT-PCR analyses using pituitary and BM RNA and the primer sets identified in FIG. 1A. This graph illustrates the statistically-significant difference (p<0.01) in gene expression when detected using upstream primer sequences targeted to exons 3 and 4 vs. when using upstream primer sequences targeted to exon 5. The location of the primers used for PCR amplification are as shown in FIG. 1A.

FIG. 1C shows the sequence of the 5′ RACE product (SEQ ID NO: 9) generated with the 5′ RACE oligo (SEQ ID NO: 22) and the downstream TSHβ gene specific primer (GSP: underlined) (SEQ ID NO: 23). Nucleotides from 60 to 185 are located in intron 4 in the portion that immediately precedes exon 5. Exon 5 begins with nucleotide 186. The 27 nucleotides that precede exon 5 encode a putative 9 amino acids signal peptide (shown by the single-letter amino acid codes). This segment is in-frame with exon 5.

FIG. 1D shows an alignment obtained in a BLAST analysis of the 5′ RACE sequence, revealing complete identity to the mouse TSHβ gene in portions of intron 4 and exon 5. The splice variant nucleotides that encode the signal peptide begin with nucleotide 159 (Query sequence). The coding sequence of the splice variant begins with nucleotide 186 (Query sequence), which is the beginning of exon 5.

FIG. 2 illustrates that native mouse TSHβ is expressed at high levels in the pituitary but not in the bone marrow or in the thyroid, whereas the novel TSHβ splice variant is expressed in all three tissues.

FIG. 2A shows that PCR combined with agarose gel electrophoresis analysis of RNA from pituitary, bone marrow, and thyroid tissue. The results illustrate that the use of a 470 primer set that spans the TSHβ coding region (SEQ ID NOS: 13 and 14) yields a product that was evident only from pituitary RNA. In contrast, when using a PCR primer set designed to detect the novel TSHβ splice variant (see FIG. 5, primer S1a, SEQ ID NOS: 24 and 25), a PCR product of the anticipated size was obtained using RNA from all three tissues.

FIG. 2B shows the results of a quantitative RT-PCR analysis used to compare the ratio of the PCR product identified using the 470 primer set with the novel splice variant product of pituitary/bone marrow (BM) and pituitary/thyroid. Using the 470 primer set (SEQ ID NOS: 14 and 14), there was an extremely high preference for expression of native TSHβ in the pituitary relative to the BM and thyroid. However, the use of the PCR primer set designed to detect the novel TSHβ splice variant (SEQ ID NOS: 24 and 25) yielded a substantially lower ratio of pituitary/BM and pituitary/thyroid.

FIG. 2C shows the results of a one-step PCR reaction used to confirm that amplification using the primer set designed to detect the novel TSHβ splice variant (SEQ ID NOS: 24 and 25) was not the result of the amplification of genomic DNA sequences. In this one-step PCR reaction, reverse transcriptase was either included or omitted. Amplification occurred only in the presence of reverse transcriptase, thus excluding the possibility that contaminating genomic DNA was present and was responsible for the detected novel TSHβ splice variant in the reactions.

FIG. 2D shows the results of another one-step PCR reaction used to rule out the presence of contaminating genomic DNA. In these reactions, four upstream primers targeted to introns 1-4 were used (see FIG. 5, primer S1b, SEQ ID NOS: 26-29). Amplification with these intron primers along with the 98-3′ reverse primer (SEQ ID NO: 21) and genomic DNA yielded four PCR products of the anticipated sizes. In contrast, when BM RNA was used instead of genomic DNA in the same experiment, PCR products were obtained only by intron primers 1 and 2 (SEQ ID NOS: 28 and 29), both of which target a region near the 5′ RACE start site (see lower panel). These findings further confirm that the data shown in FIGS. 1C and 1D accurately reflect the 5′ RACE start site.

FIG. 3 illustrates the amino acid composition of the novel mouse TSHβ splice variant (SEQ ID NOS: 3 and 4).

FIG. 3A shows the predicted amino acid sequence of the novel TSHβ slice variant, consisting of a first nine amino acids MLRSLFFPQ, which make up the signal peptide, and the remaining eighty-four amino acids, which make up the splice variant polypeptide.

FIG. 3B shows the location of the novel TSHβ isoform (underlined residues) within the 118 amino acid sequence of the full-length native TSHβ molecule (both non-underlined and underlined residues; SEQ ID NO: 2, residues 21-138).

FIG. 3C shows a secondary structure analysis of the novel TSHβ polypeptide, indicating the hydrophobic momentum index, the transmembrane helix momentum, and the beta preference indices. Note the high hydrophobic momentum index and the high transmembrane helix momentum of the first 7-9 amino acids, which comprise the signal peptide.

FIG. 3D shows that TSHβ is secreted into the media from CHO cells transfected with native or novel splice variant TSHβ constructs, indicating that both forms of TSHβ are produced as secreted proteins. Control CHO cells transfected with LacZ had no detectable TSHβ. Data are mean values±SEM of three replicate samples.

FIG. 3E shows TSHβ immunoprecipitation analyses from various cell lysates using antibody that binds TSHβ. Cell lysates from non-transfected CHO cells were non-reactive by immunoprecipitation. Immunoprecipitation of cell lysates from CHO cells transfected with the native TSHβ construct identified a 17 kDa product. Immunoprecipitation of lysates of CHO cells transfected with the novel TSHβ construct identified an 8 kDa product.

FIG. 4 illustrates that recombinant mouse novel TSHβ splice variant is capable of delivering a cAMP signal and is upregulated in the thyroid following systemic virus infection.

FIG. 4A shows the cAMP response of an alveolar macrophage cell line (AM cells) cultured with log₁₀ dilutions of recombinant native TSHβ, novel splice variant TSHβ, media (negative control), or forskolin (positive control) at the concentration indicated. Both native and variant forms of TSHβ elicited a cAMP response in a dose-dependent fashion. (*p<0.05 compared to other molar concentrations for that form of TSH). Data are mean values±SEM of four replicate samples.

FIG. 4B shows cAMP response of FRTL-5 cells. The cells were seeded into 24 well plates as described in the examples below, were cultured with log₁₀ dilutions of recombinant native TSHβ, splice variant TSHβ, media (negative control), or forskolin (positive control) at the concentrations indicated. Both native and variant forms of TSHβ elicited a cAMP response in a dose-dependent fashion. (*p<0.01 compared to other molar concentrations for that form of TSH). Data are mean values±SEM of three replicate samples.

FIG. 4C shows the results of a quantitative RT-PCR analysis of RNA from thyroid tissues 48 hrs post-reovirus infection using the 470 and novel primer sets (SEQ ID NOS: 13-14 and 24-25). Note the statistically-significant increase in the novel TSHβ splice variant gene expression in the thyroid of infected mice compared to the thyroid of non-infected mice, and the lack of change in the thyroid in gene expression of native TSHβ in the thyroid during virus infection as identified with the 470 primers. Data are mean values±SEM of three replicate values. In each case, gene expression of virus infected mice was compared to that of non-infected mice, the latter being designated as a gene expression level of 1.0.

FIG. 5 illustrates the specific positions of primer sequences. S1a refers to the location of the primer set designed to detect the novel TSHβ splice variant. This primer set consists of a 24 nucleotide upstream primer targeted to a region within intron 4 (SEQ ID NO: 24) and a downstream primer sequence located just after the TAA TSHβ stop codon used in FIG. 2A (SEQ ID NO: 25). S1b refers to the location of the four upstream primer sequences designed to detect introns 1-4 and used for the experiment in FIG. 2D (SEQ ID NOS: 26-29).

FIG. 6 illustrates gene expression levels of native and novel TSHβ in various human tissues as determined by conventional PCR.

FIG. 6A shows agarose gel analysis of PCR-amplified transcripts obtained from human pituitary, thyroid, PBL, and bone marrow RNAs using human native or novel primer sets (SEQ ID NOS: 30-32). PCR-amplified products of both native and novel TSHβ were detected in pituitary RNA. However, only novel TSHβ message was detected in thyroid and PBL RNA. Neither native nor novel TSHβ products were detected in bone marrow RNA.

FIG. 6B shows that the TSHβ gene was expressed in the pituitary, the thyroid, and PBL, but not bone marrow.

FIG. 6 C shows that 18s gene expression levels were equivalent in all four samples.

FIG. 6 C shows that 18s gene expression levels were equivalent in all four types of tissue samples.

FIG. 7 illustrates gene expression levels of native and novel human TSHβ in various tissues as determined by quantitative real-time PCR (qRT-PCR) using the human native or novel primer sets (SEQ ID NOS: 30-32).

FIG. 7A shows qRT-PCR results revealing high levels of both native and novel human TSHβ transcript in pituitary RNA. In contrast, only the novel variant TSHβ transcript was detected in thyroid and PBL RNA. Neither native nor novel TSHβ transcript were detected in bone marrow (BM) RNA.

FIG. 7B shows qRT-PCR results identifying expression levels of TSHα in various tissues. High levels of expression of TSHα were detected in the pituitary, modest levels of expression were detected in the thyroid, and low levels of expression were detected in PBL. No TSHα gene expression was detected in BM. Data are mean values±SEM of three replicate samples.

FIG. 8 shows a result from a BLAST sequence analysis of a PCR-amplified product of novel human TSH from thyroid RNA. The amplified sequence (Query, SEQ ID NO: 10) was compared to the known human TSHβ sequence (Sbjct). The underlined nucleotides are the twenty-seven nucleotides in human intron 2 that are in-frame with exon 3, and which begin with an ATG start codon. The three nucleotides (ATT) prior to the twenty-seven nucleotides are the first three nucleotides of the upstream novel TSHβ primer (SEQ ID NO: 32). The non-underlined nucleotides correspond to human exon 3 down to the TAA stop codon.

FIG. 9 illustrates the relationship between nucleotide and amino acid sequences of human and mouse novel splice variant TSHβ.

FIG. 9A shows a nucleotide sequence alignment of the human (top rows, SEQ ID NO:7) and mouse (bottom rows, SEQ ID NO: 3) TSHβ splice variant. Underlined nucleotides code for the putative human and mouse signal peptides that are encoded by human intron 4 and mouse intron 2, respectively. The non-underlined nucleotides are encoded by human exon 3 or mouse exon 5, of TSHβ, respectively.

FIG. 9B shows a comparison of the amino acid sequences of the signal peptide of the human and mouse TSHβ splice variants (SEQ ID NOS: 8 and 4, respectively), indicating differences at amino acid positions three and four.

FIG. 10 illustrates that murine TSHβ splice variant expression can be suppressed using siRNA. The mouse alveolar macrophage (AM) cell line was transfected with the pSilencer™ 4.1-CMV puro expression vector containing the construct shown in FIG. 12B. Results show that TSHβ splice variant expression was suppressed in AM transient transfectants (48 hrs) and stable transfectants (4 wks), relative to TSHβ gene expression in mock-transfected cells at 4 wks. Results also show that stable transfectants had normal expression levels of 18s.

FIG. 11 illustrates that murine TSHβ splice variant recombinant protein suppresses circulating levels of the thyroid hormone, T4. Mice were injected either with PBS (N=5) or the mouse TSHβ splice variant recombinant protein (N=4) for 3 days. Serum T4 levels were measured 24 hours later. Note the statistically-significant (p<0.001) suppression in circulating T4 levels in mice injected with the TSHβ splice variant protein relative to animals injected with PBS.

FIG. 12 shows a siRNA construct used to selectively suppress expression of the TSHβ splice variant.

FIG. 12A shows the location of the 21 nucleotide sequence (underlined) used to make the mouse siRNA for suppression of the TSHβ splice variant (SEQ ID NO: 3, residues 9-29). The first 27 nucleotides are located in mouse intron 4; the remaining nucleotides make up mouse exon 5. 20 of the 21 nucleotides for the siRNA sequence (underlined) are from intron 4; the last nucleotide is from the start of exon 5.

FIG. 12B shows the template for the two strands of the shRNA oligos to be used with the pSilencer™ 4.1-CMV puro expression vector for generating an shRNA used for RNA-inhibition of the TSHβ splice variant (SEQ ID NOS: 11-12). This construct was used to obtain the results shown in FIG. 10.

### DETAILED DESCRIPTION OF EXEMPLARY EMBODIMENTS

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

### DEFINITIONS

As used herein, and unless otherwise indicated, the terms “treat”, “treating”, and “treatment” contemplate an action that occurs while a patient is suffering from TSHβ-related disorders that reduces the severity of one or more symptoms or effects of TSHβ-related disorders, or a related disease or disorder. Where the context allows, the terms “treat”, “treating”, and “treatment” also refers to actions taken toward ensuring that individuals at increased risk of TSHβ-related disorders are able to receive appropriate surgical and/or other medical intervention prior to onset of TSHβ-related disorders. As used herein, and unless otherwise indicated, the terms “prevent”, “preventing”, and “prevention” contemplate an action that occurs before a patient begins to suffer from TSHβ-related disorders that delays the onset of, and/or inhibits or reduces the severity of, TSHβ-related disorders. As used herein, and unless otherwise indicated, the terms “manage”, “managing”, and “management” encompass preventing, delaying, or reducing the severity of a recurrence of TSHβ-related disorders in a patient who has already suffered from such a disease or condition. The terms encompass modulating the threshold, development, and/or duration of the TSHβ-related disorders or changing how a patient responds to the TSHβ-related disorders.

As used herein, and unless otherwise specified, a “therapeutically effective amount” of a compound is an amount sufficient to provide any therapeutic benefit in the treatment or management of TSHβ-related disorders or to delay or minimize one or more symptoms associated with TSHβ-related disorders. A therapeutically effective amount of a compound means an amount of the compound, alone or in combination with one or more other therapies and/or therapeutic agents that provides any therapeutic benefit in the treatment or management of TSHβ-related disorders, or related diseases or disorders. The term “therapeutically effective amount” can encompass an amount that alleviates TSHβ-related disorders, improves or reduces TSHβ-related disorders, improves overall therapy, or enhances the therapeutic efficacy of another therapeutic agent.

As used herein, and unless otherwise specified, a “prophylactically effective amount” of a compound is an amount sufficient to prevent or delay the onset of TSHβ-related disorders, or one or more symptoms associated with TSHβ-related disorders or prevent or delay its recurrence. A prophylactically effective amount of a compound means an amount of the compound, alone or in combination with one or more other treatment and/or prophylactic agent that provides a prophylactic benefit in the prevention of TSHβ-related disorders. The term “prophylactically effective amount” can encompass an amount that prevents TSH-related disorders, improves overall prophylaxis, or enhances the prophylactic efficacy of another prophylactic agent.

Thyroid Stimulating Hormone

Human and mouse TSHβ nucleic acid sequences for use in the present invention include, but are not limited to, those described below. The inventors have discovered that previously unrecognized splice variants of mammalian TSHβ (aliases include thyroid stimulating hormone beta-subunit, thyrotropin beta-subunit, TSHB, TSHB-beta, TSHBB CHNG4, TSH-BETA, OTTHUMP00000013654; and thyrotropin beta subunit) are expressed in both humans and mice (SEQ ID NOS: 4 and 8).

The native human TSHβ gene is described, among other places, in SEQ ID NO: 5; Accession Nos: AH003629 (Homo sapiens thyrotropin beta-subunit (TSHBB) gene); M23669 (Homo sapiens thyrotropin beta-subunit (TSHBB) gene, exon 1 and promoter region); M23671 (Homo sapiens thyrotropin beta-subunit (TSHBB) gene, exon 3); M23670 (Homo sapiens thyrotropin beta-subunit (TSHBB) gene, exon 2); NM_{—}000549 (Homo sapiens thyroid stimulating hormone, beta (TSHBB), mRNA); AH001548 (Human thyrotropin beta (TSHB-beta) subunit gene); M21024 (Human thyrotropin beta (TSHB-beta) subunit gene, exons 2 and 3); M21023 (Human thyrotropin beta (TSHB-beta) subunit gene, exon 1); BC069298 (Homo sapiens thyroid stimulating hormone, beta, mRNA (cDNA clone MGC: 97444 IMAGE: 7262720), complete cds); 551112 (thyrotropin beta subunit [human, lymphocytes, mRNA Partial, 262 nt]); NM_{—}000549 (Homo sapiens thyroid stimulating hormone, beta (TSHB), mRNA); among others is present on human chromosome: 1; Location: 1p13 (annotated as Chromosome 1, NC_{—}000001.10 (115572415 . . . 115576941), MIM: 188540, GeneID: 7252).

The mouse (Mus musculus) ortholog of the human TSHβ gene, (also known as, among other things as mouse thyrotropin beta-subunit, TSHB-beta, Tshb thyroid stimulating hormone, beta subunit [Mus musculus], MGC151206, MGC151208 and thyrotropin) is described, among other places in SEQ ID NO: 1; Accession Nos: AH002108 (Mouse thyrotropin beta-subunit (TSHB-beta) gene); M22740 (Mouse thyrotropin beta-subunit (TSHB-beta) gene, exons 3 and 4, complete cds, clone lambda-TSHB-beta); M22739 (Mouse thyrotropin beta-subunit (TSHB-beta) gene, exons 1, 2 and 3, clone lambda-TSHB-beta); M54943 (Mouse thyroid stimulating hormone beta-subunit (TSHB-beta) mRNA, complete cds); DQ275152 (Mus musculus strain B6.NODc3 homeodomain interacting protein kinase 1 (Hipk1) mRNA, partial cds); BC144732.1 (Mus musculus thyroid stimulating hormone, beta subunit, mRNA (cDNA clone MGC:178288 IMAGE:9053280), complete cds); BC116829 (Mus musculus thyroid stimulating hormone, beta subunit, mRNA (cDNA clone MGC:151206 IMAGE:40126148), complete cds); BC116831 (Mus musculus thyroid stimulating hormone, beta subunit, mRNA (cDNA clone MGC:151208 IMAGE:40126150), complete cds); NM_{—}009432 (Mus musculus thyroid stimulating hormone, beta subunit (Tshb), mRNA); J00644 (mouse thyrotropin beta subunit (TSHB-beta) mRNA); and is located on Chromosome: 3; Location: 3 48.5 cM (Annotation: Chromosome 3, NC_{—}000069.5 (102581321 . . . 102586637, complement), GeneID: 22094).

Additional animal orthologs of human TSHβ have been identified, predicted and described, including for example, those of other primates, including but not limited to, the common chimpanzee (Pan troglodytes) and the Rhesus Macaque (Macaca mulatta). Nucleic acid sequences encoding for chimpanzee TSHβ are provided, at least in GenBank™ accession numbers: NC_{—}006468 (Pan troglodytes chromosome 1, reference assembly (based on Pan troglodytes-2.1); NW_{—}001229571 (Pan troglodytes chromosome 1 genomic contig, reference assembly (based on Pan troglodytes-2.1)); XM_{—}001160337 (PREDICTED: Pan troglodytes similar to Thyrotropin beta chain precursor (Thyroid-stimulating hormone subunit beta) (TSHB-beta) (TSHB-B) (Thyrotropin alfa) (LOC748248), mRNA). Chimpanzee TSHβ is encoded on chromosome: 1 (Annotation: Chromosome 1, NC_{—}006468.2 (122615156 . . . 122619923, complement), GeneID: 748248. The nucleic acid sequences encoding for Rhesus Macaque TSHβ are provided, at least in GenBank™ accession number XM_{—}001111873 (PREDICTED: Macaca mulatta similar to Thyrotropin beta chain precursor (Thyroid-stimulating hormone beta subunit) (TSHB-beta) (TSHB-B) (Thyrotropin alfa) (TSHBB), mRNA). Rhesus TSHβ is encoded on chromosome: 1 (Annotation: Chromosome 1, NC_{—}007858.1 (118007928 . . . 118012278), GeneID: 709374).

The nucleic acid sequences encoding rat, Rattus norvegicus, TSHβ have also been described and are provided, at least in GenBank™ accession numbers: M13897 (Rattus norvegicus thyrotropin beta subunit (TSHB-beta) gene, complete cds, clones RP100-14 and RP21); AC_{—}000070 (Rattus norvegicus chromosome 2, alternate assembly (based on Celera), whole genome shotgun sequence); NM_{—}013116 (Rattus norvegicus thyroid stimulating hormone, beta (TSHBβ), mRNA); NM_{—}053777 (Rattus norvegicus mitogen-activated protein kinase 8 interacting protein 1 (Mapk8ip1), mRNA); D00578 (Rattus norvegicus mRNA for TSHB beta subunit, partial cds); NC_{—}005101 (Rattus norvegicus chromosome 2, reference assembly (based on RGSC v3.4)); NW_{—}001084807 (Rattus norvegicus chromosome 2 genomic contig, alternate assembly (based on Celera assembly), whole genome shotgun sequence); NW_{—}047627 (Rattus norvegicus chromosome 2 genomic contig, reference assembly (based on RGSC v3.4)); X01454 (Rat mRNA for thyrotropin-beta (TSHB) sequence), AH003533 (Rat thyrotropin (TSHB) beta-subunit gene); BC058488 (Rattus norvegicus thyroid stimulating hormone, beta, mRNA (cDNA clone MGC:72898 IMAGE:6921292), complete cds; M14450 (Rat thyrotropin (TSHB) beta-subunit gene, exons 2 and 3); M14499 (Rat thyrotropin (TSHB) beta-subunit gene, exon 1), M10902 (Rat thyrotropin-beta-subunit (TSHB-Beta) mRNA, complete cds)); and is encoded on chromosome: 2; Location: 2q34 (Annotation: Chromosome 2, NC_{—}005101.2 (197908308 . . . 197913186, complement), GeneID: 25653).

The nucleic acid sequences encoding dog, Canis familiaris, TSHβ have also been described and are provided, at least in GenBank™ accession number U51644 (Canis familiaris thyrotropin beta chain mRNA, complete cds); and NM_{—}001003290 (Canis lupus familiaris thyroid stimulating hormone, beta (TSHB), mRNA) and are encoded on chromosome: 17 (Annotation: Chromosome 17, NC_{—}006599.2 (55759381.55760269), GeneID: 403973). The nucleic acid sequences encoding cat, Felis catus, TSHβ, includes, but is not limited to NM_{—}001048015 (cat: thyroid stimulating hormone, beta (TSHB), mRNA), betaGene ID: 554350).

The nucleic acid sequences encoding swine, Sus scrofa, TSHβ have also been described and are provided, at least in GenBank™ accession numbers: NC_{—}010446 (Sus scrofa chromosome 4, reference assembly (based on Sscrofa5), complete sequence); NW_{—}001886257 (Sus scrofa chromosome 4 genomic contig, reference assembly (based on Sscrofa5), complete sequence); NM_{—}214368 (Sus scrofa thyrotropin beta subunit (TSHB-BETA), mRNA); U39816 (Sus scrofa thyrotropin beta subunit precursor (TSHB-beta) mRNA, complete cds)); and is encoded on chromosome: 4 (Annotation: Chromosome 4, NC_{—}010446.1 (90531104 . . . 90532043, complement), GeneID: 397658).

The nucleic acid sequences encoding horse, Equus caballus, TSHB have also been described and are provided, at least in GenBank™ accession numbers: NC_{—}009148 (Equus caballus chromosome 5, reference assembly (based on EquCab2), whole genome shotgun sequence); NM_{—}001082491 (Equus caballus thyrotropin beta chain (TSHB-BETA), mRNA); NW_{—}001867420 (Equus caballus chromosome 5 genomic contig, reference assembly (based on EquCab2), whole genome shotgun sequence); U51789 (Equus caballus thyrotropin beta chain (TSHB-beta) mRNA, complete cds)); NM_{—}001082491 (Equus caballus thyrotropin beta chain (TSH-BETA), mRNA) and is encoded on chromosome: 5 (Annotation: Chromosome 5, NC_{—}009148.2 (53762959.53763858, complement), GeneID: 100034188).

Additional mammalian orthologs of TSHβ nucleic acid sequences for use in the present invention include, but are not limited to, those described in, for example, GenBank™ accession numbers: NM_{—}001163072 (European rabbit: Oryctolagus cuniculus thyroid stimulating hormone, beta (TSHB), mRNA), EU562212 (Oryctolagus cuniculus thyroid-stimulating hormone beta subunit (Tshb) mRNA, complete cds), GeneID: 100302414; AY048589 (Gray Short-tailed Opossum: Monodelphis domestica thyroid stimulating hormone beta subunit precursor, mRNA, complete cds); NM_{—}174205.1 (domestic cattle: Bos taurus thyroid stimulating hormone, beta (TSHB), mRNA, and is encoded on Chromosome: 3, NC_{—}007301.3 (30779288.30784523, complement), GeneID: 281552.

The TSHβ-related diseases and disorders prevented, treated or reduced by the methods and compositions disclosed herein also occur in other mammals. The word mammal means any mammal that is susceptible to TSHβ-related disorders. Some examples of such mammals include, for example, companion animals, such as, but not limited to, dogs and cats; farm animals, such as, but not limited to, horses, pigs, cattle, sheep and goats; laboratory animals, such as, but not limited to, mice, rats, hamsters, rabbits and guinea pigs; animals used in sports, such as, but not limited to, horses and dogs; primates, such as, but not limited to, monkeys, apes, chimpanzees and humans. In some embodiments, humans are preferably treated according to a method disclosed herein and/or using a composition disclosed herein and in others, non human mammals are the subject of such therapies.

The function, structure and uses for TSH are well known to persons of ordinary skill in the art and have been described (Emerson C H, Torres M S. Recombinant human thyroid-stimulating hormone: pharmacology, clinical applications and potential uses. BioDrugs 2003; 17(1):19-38, 2003; Kelly G S. Peripheral metabolism of thyroid hormones: a review. Altern Med Rev 2000; 5(4):306-33; Szkudlinski M W, Fremont V, Ronin C, Weintraub B D. Thyroid-stimulating hormone and thyroid-stimulating hormone receptor structure-function relationships. Physiol Rev 2002; 82(2):473-502).

Recombinant human TSH is available from Genzyme as Thyrogen® (Genzyme Corporation, Cambridge, Mass.) The alpha and beta subunits of Thyrogen® are identical to those of human pituitary TSH. Thyrogen® has been approved for use in humans by the United States Food and Drug Administration. Thyrogen® is used to prepare thyroid cancer patients for radio-ablation. Thyrogen® is also used in thyroid cancer patients who have been treated by thyroidectomy and radio-ablation but are at risk of harboring residual thyroid cancer. These thyroidectomised thyroid cancer patients are unable to secrete pituitary TSH upon thyroid hormone withdrawal and therefore Thyrogen® is used to prepare them for whole body iodide scans and serum Tg measurements.

TSH has also been the subject of many patent applications and several issued U.S. patents. For example, U.S. Pat. No. 5,177,193 entitled “Modified forms of reproductive hormones” (hereinafter “the '193 patent”) describes recombinant native and mutant forms of human reproductive hormones with characteristic glycosylation patterns which are influential in the metabolic activity of the protein. The invention in the '193 patent also provides recombinant mutant forms of the human alpha subunit common to FSH, LH, CG, and TSH, to obtain hormones which also have unique glycosylation patterns. Also provided in the '193 patent are recombinant materials to produce these subunits separately or together to obtain complete heterodimeric hormones of regulated glycosylation pattern and activity. Modified forms of LH and FSH beta subunits which enhance the rate of dimerization and secretion of the dimers or individual chains are also disclosed in the '193 patent.

U.S. Pat. No. 6,455,282 entitled “Cells, vectors and methods for producing biologically active TSH” (hereinafter “the '282 patent”) describes a biologically active heteropolymeric proteins composed of a plurality of subunits, both subunits being synthesized in a single cell having an expression vector comprising heterologous DNA encoding the subunits. Preferably, the protein in the '282 patent is similar to the human or ungulate fertility hormones, LH and FSH.

U.S. Pat. No. 7,479,549, entitled “Recombinant canine thyroid stimulating hormone and methods of production and use thereof” (hereinafter “the '549 patent”) describes a nucleic acid having a sequence at least 98% homologous to a sequence which encodes the α subunit of canine thyroid stimulating hormone (TSH). The invention in the '549 patent also includes a nucleic acid having a sequence at least 98% homologous to a sequence which encodes the β subunit of canine TSH. The invention in the '549 patent also includes a method of producing recombinant canine thyroid stimulating hormone (rcTSH) subunit by expressing the sequences in a transgenic insect cell modified to silylate proteins and producing a sialylated rcTSH subunit for use in the diagnosis and treatment of canine hypothyroidism.

United States Patent Application Publication No. 2003/0009778, entitled “Transgenic mice containing thyroid stimulating hormone receptor (TSH-R) gene disruptions” (hereinafter “the '778 application”) describes transgenic animals, as well as compositions and methods relating to the characterization of TSH-R gene function. Such transgenic mice in the '778 application are useful as models for disease and for identifying agents that modulate gene expression and gene function, and as potential treatments for various disease states and disease conditions.

United States Patent Application Publication No. 2004/0176294 entitled “Use of thyroid-stimulating hormone to induce lipolysis” describes the use of thyroid-stimulating hormone (TSH) to induce lipolysis, treat various diseases such as obesity, insulin resistance, liver steatosis, hyperlipidemia, and type-2 diabetes.

United States Patent Application Publication No. 2007/0010446, entitled “Methods for Treating Inflammation Using Thyroid Stimulating Hormone” (hereinafter “the '446 application”) describes anti-inflammatory activity by thyroid stimulating hormone. Polypeptides of thyroid stimulating hormone described in the '446 application have a novel use as an anti-inflammatory agent as a stand-alone therapy, or in conjunction with other anti-inflammatory agents. In addition, thyroid stimulating hormone can be used to potentiate the anti-inflammatory activity of glucocorticoid treatment.

United States Patent Application Publication No. 2008/0107605, entitled ‘Biologically active synthetic thyrotropin and cloned gene for producing same” (hereinafter “the '605 application”) describes substantially pure recombinant TSH prepared from a clone comprising complete nucleotide sequence for the expression of the TSH. Diagnostic and therapeutic applications of the synthetic TSH are described in the '605 application.

However, none of the above-cited references teaches or suggests the discovery and characterization of previously unrecognized polynucleotides and amino acids encoding splice variants of TSHβ. Furthermore, the above-cited references do not teach or suggest the application of the TSHβ splice variants for the diagnosis, drug screening, clinical trial monitoring, and treatment of TSHβ-related disorders.

TSHβ Splice Variants

In both humans and mice, the native TSHβ polypeptide consists of 138 amino acids of which 20 amino acids comprise the signal peptide and 118 constitute the mature protein. See SEQ ID NOS: 2 and 6. Overall, there is 82% homology at the nucleic acid level and 88% homology at the amino acid level between human (accession no. NM_{—}000549) and mouse (accession no. NM_{—}009432) TSH.

Mouse Variant TSHβ

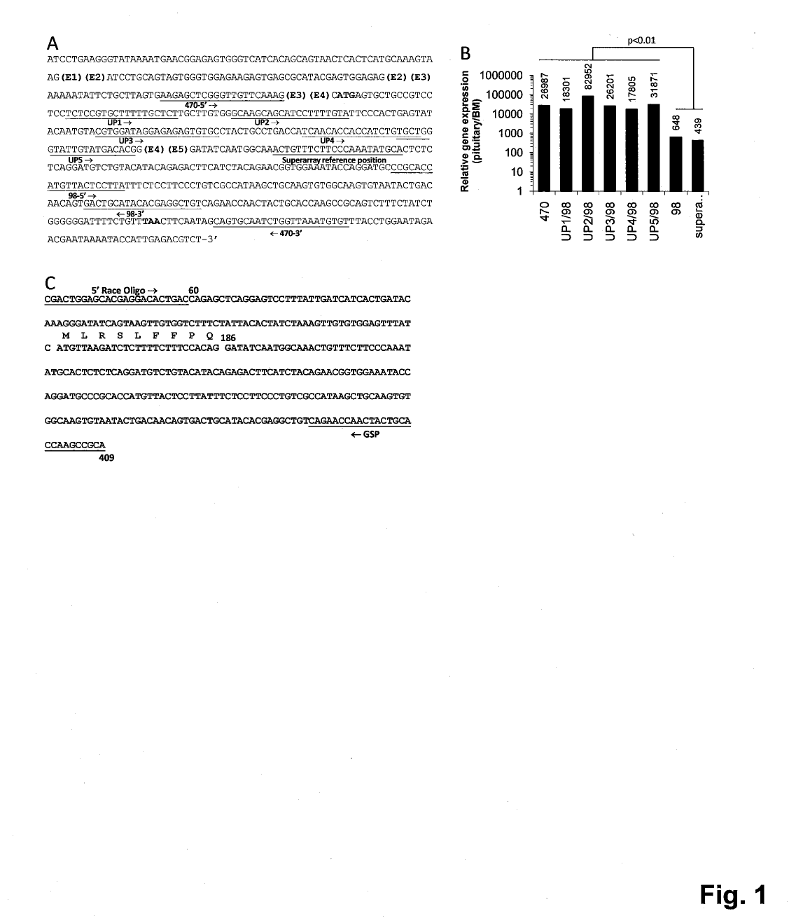

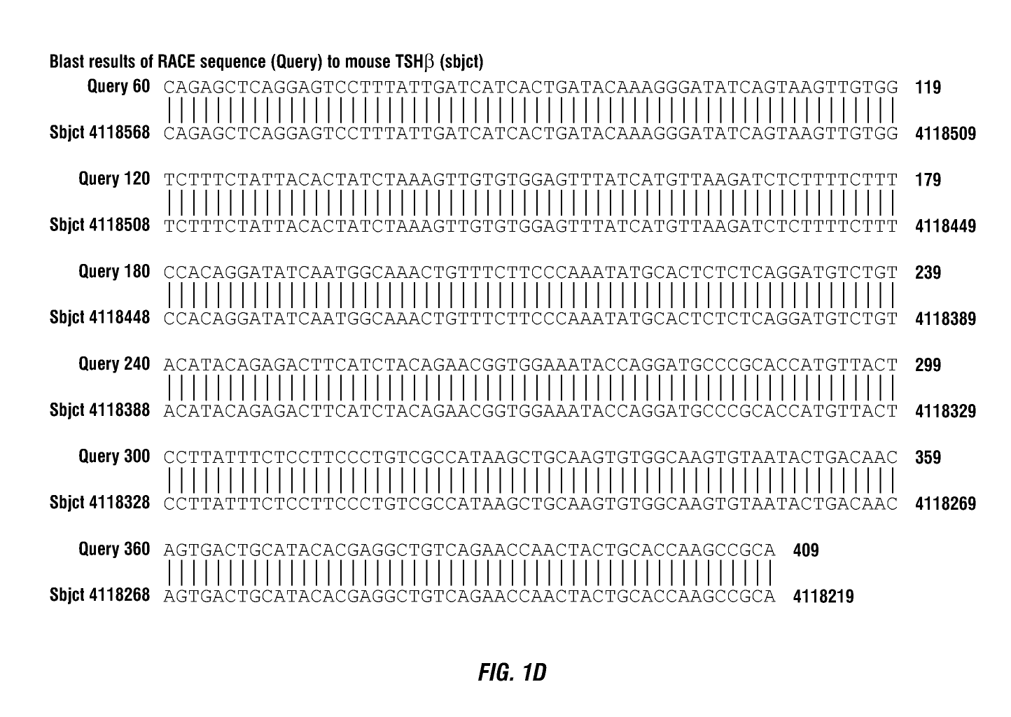

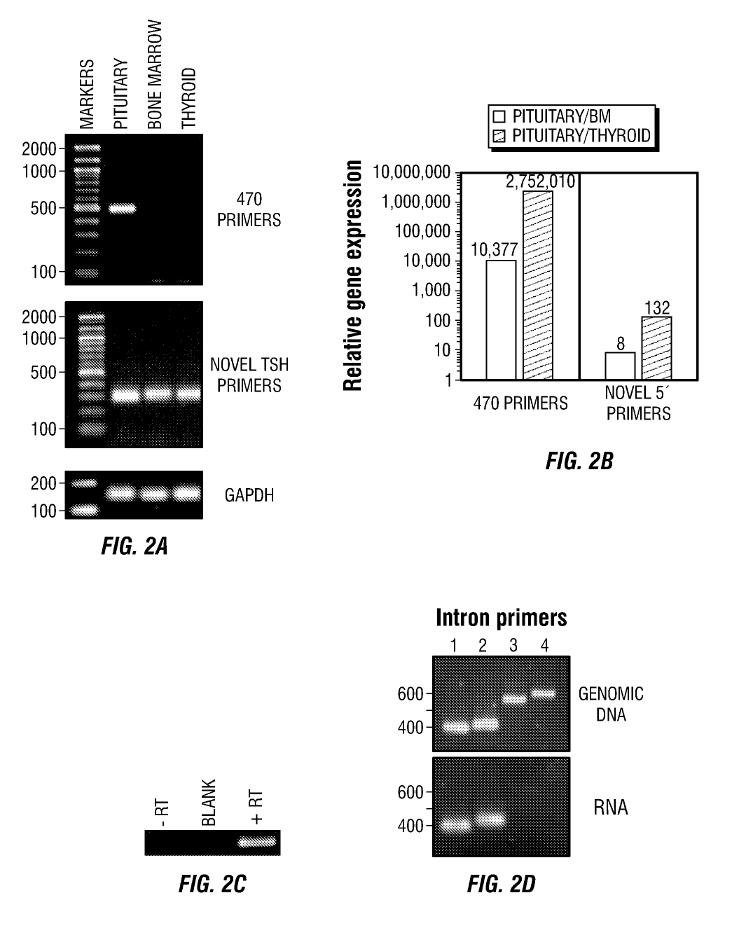

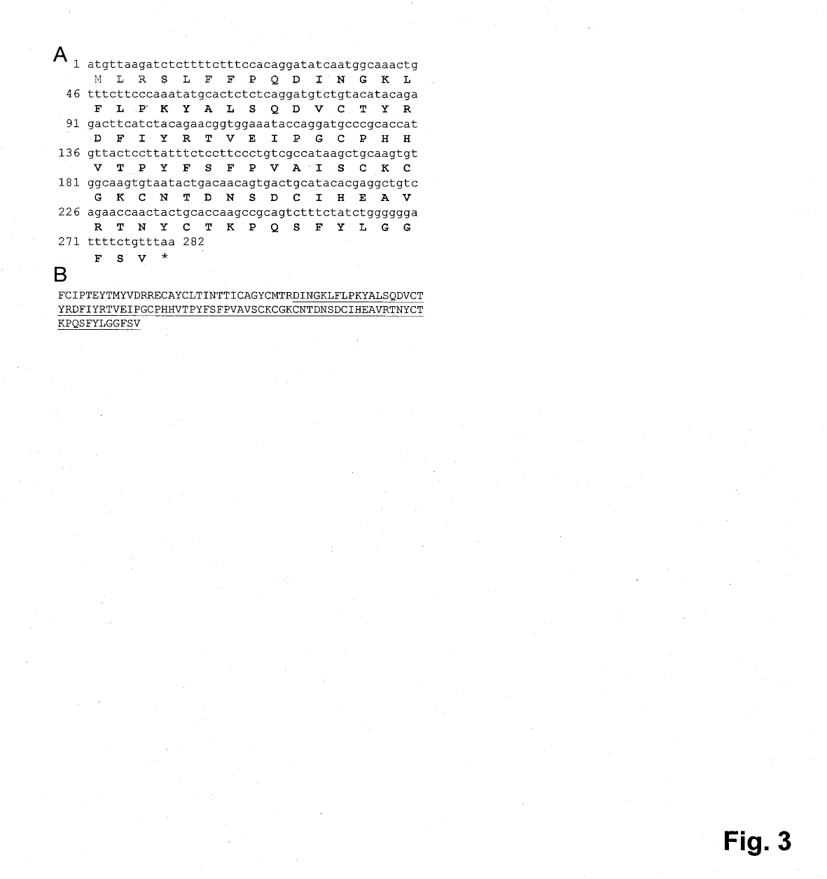

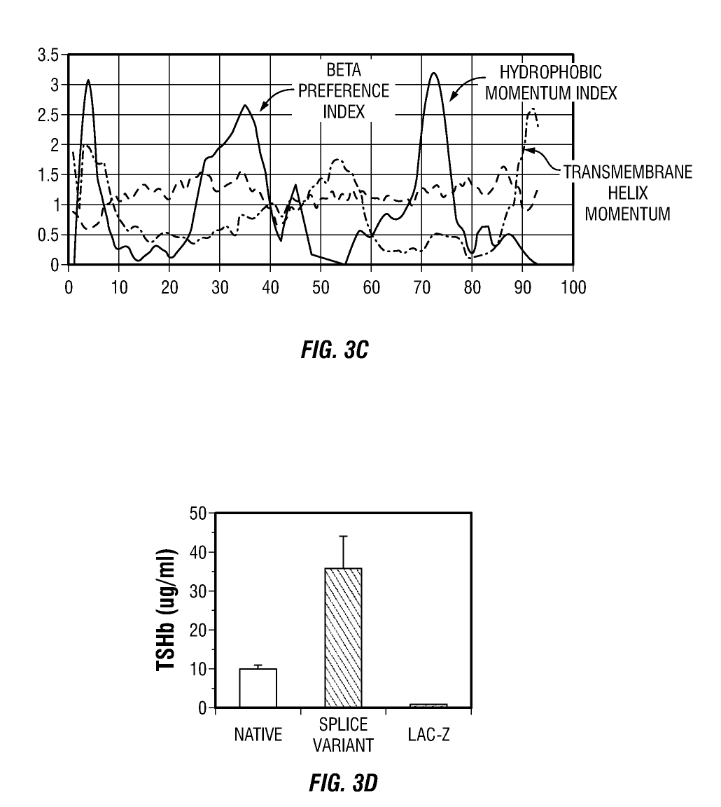

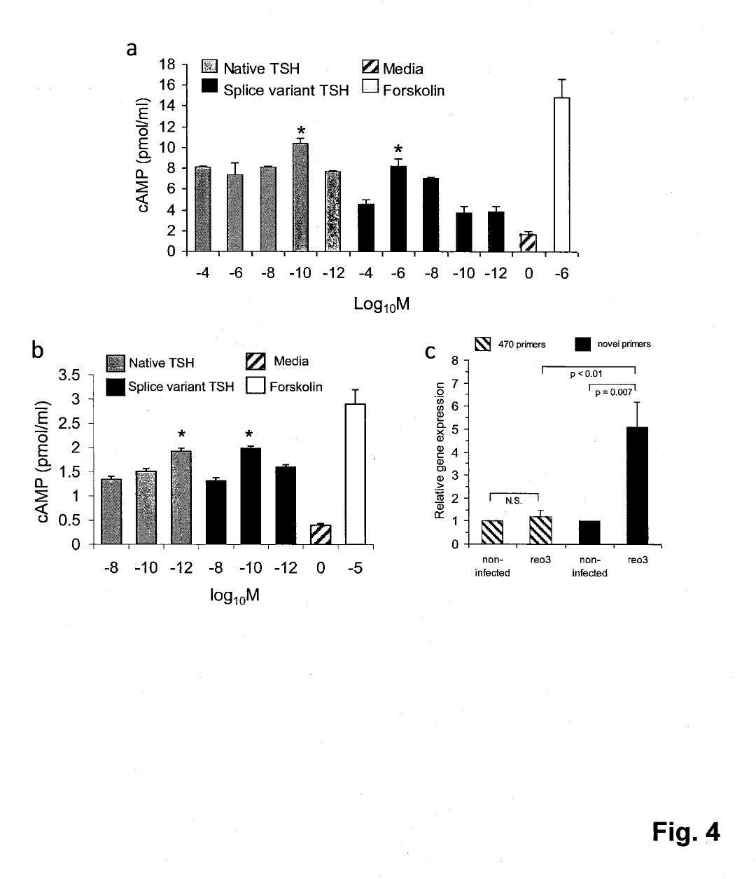

As illustrated in FIG. 1A, the mouse TSHβ gene consists of 5 exons, with the coding region located in portions of exon 4 and exon 5 (Gordon D F, Wood W M, Ridgway E C. Organization and nucleotide sequence of the gene encoding the beta-subunit of murine thyrotropin. DNA 1988; 7(1):17-26). In mice, using 5′ rapid amplification of cDNA, Applicants discovered that mouse bone marrow cells produce a novel in-frame TSHβ splice variant generated from a portion of intron 4 with all of the coding region of exon 5, but none of exon 4. See FIG. 1C. TSHβ splice variant consists of a twenty-seven nucleotide portion of intron 4 that is contiguous with the coding region of exon 5 of mouse TSHβ, resulting in a polypeptide that comprises 71.2% of the native TSHβ molecule. As illustrated in FIG. 2, Applicants also discovered that the TSHβ splice variant gene was expressed at low levels in the mouse pituitary but at high levels in mouse bone marrow and thyroid. By utilizing immunoprecipitation with anti-TSHβ antibody, Applicants also established that lysates of CHO cells that had been transfected with a construct containing native TSHβ expressed a 17 kDa product, while lysates from CHO cells transfected with constructs containing variant TSHβ expressed an 8 kDa product. See FIG. 3E. Applicants also discovered that a splice variant of the TSHβ protein elicited a cAMP response from FRTL-5 thyroid follicular cells and cells from a mouse alveolar macrophage cell line. See FIGS. 4A-4B. In addition, Applicants determined that the expression of the TSHβ splice variant, but not the expression of the native form of TSHβ, was significantly up-regulated in the thyroid during systemic virus infection. See FIG. 4C.

As detailed in the Examples below, a novel TSHβ splice variant was identified in hematopoietic cells from mouse bone marrow (BM) using quantitative RT-PCR (qRT-PCR). The full-length mRNA sequence of native TSHβ is shown in FIG. 1A (SEQ ID NO: 1), which indicates the positions of the five mouse TSHβ exons (designated E1-E5), with the translated portion beginning with the ATG (bolded) at the second nucleotide of exon 4. The TSHβ transcript is produced from the second nucleotide of E4 and extends to the TAA stop codon at the distal end of E5. Using PCR amplification with pituitary and bone marrow RNAs and primer sequences, which span the previously known TSHβ coding region, which were targeted to a region in exon 3 and exon 5, it was consistently observed that a marked difference (26,987 fold greater) in the amount of amplified product for pituitary vs. bone marrow RNA depending on which primer was used. See FIG. 2A. That pattern also held true using five additional upstream primers targeted to regions in exon 4 with a downstream primer targeted to exon 5. When qRT-PCR analysis was done using two primer sets targeted to exon 5, the fold difference in gene expression between pituitary vs. BM was 648 and 439, respectively. See FIG. 2B. This represented a statistically-significant (p<0.01) 62.8-fold reduction (34,019 vs. 543) in the relative gene expression of the ratio of pituitary/BM TSHβ expression using upstream primer sequences targeted to exons 3 or 4 compared to primers targeted to exon 5. Without being bound by theory, it is envisioned that the qRT-PCR differences between BM and pituitary RNAs as a function of the primer target location was the result of alternative splicing of the TSHβ gene at or near the junction of exons 4 and 5.

By performing 5′ RACE analysis using a highly-purified preparation of BM RNA to obtain the sequence of bone marrow TSHβ mRNA, a sequence was consistently obtained that is shown in FIG. 1C (SEQ ID NOS: 3 and 9). The underlined nucleotide regions are the 5′ RACE oligo (FWD) and the 3′ TSHβ GSP (SEQ ID NOS: 22 and 23, respectively). A gene BLAST™ search revealed complete homology to a portion of the mouse TSHβ gene. A striking finding from these studies was that all of the 5′ RACE sequences obtained from BM RNA included a portion of intron 4 that was contiguous with exon 5 FIGS. 1C and 1D. A potential ATG (methionine) start codon is followed by a sequence that codes for 9 amino acids (MLRSLFFPQ) that are in-frame with TSHβ exon 5 beginning at nucleotide 186. Thus, an open reading frame was identified that contains an ATG and a Kozak sequence consisting of the ATC prior to the ATG triplet. Without being bound by theory, these data indicate a modified splicing mechanism for BM TSHβ, and explain the low levels in PCR product from bone marrow RNA using upstream primer sequences targeted to exons 3 or 4 vs. the abundance of product using primers targeted to exon 5.

The physiochemical characteristics of the variant TSHβ polypeptide predicted from the nucleotide sequence is shown in FIG. 3 (SEQ ID NO: 3). This polypeptide consists of a 9 amino acid leader sequence followed by an eighty-four amino acid polypeptide of the mature protein molecule coded for by exon 5 up to the TSHβ stop codon (FIG. 3A; SEQ ID NO: 4). The difference between the splice variant TSHβ polypeptide (FIG. 3B, underlined amino acids) and the native TSHβ molecule (FIG. 3B, both non-underlined and underlined amino acids) is the lack of amino acids coded for by exon 4 (nonunderlined amino acids). The secondary structure of the novel TSHβ splice variant is shown in FIG. 3C, which shows a high hydrophobic moment index and a high transmembrane helix preference for the first 9 amino acids, and thus favoring a transmembrane location and a likely signal peptide function. Cell-free supernatants from CHO cells transfected with native and splice variant TSHβ constructs contained high levels of TSHβ as detected with an anti-mouse TSHβ specific monoclonal antibody (FIG. 3D). The currently described novel isoform variant of mouse TSHβ results from the retention of a portion of intron 4. Splice variants that incorporate intronal pieces possibly occur in upwards of 15% of human and mouse genes. However, most such splicing events are associated with disease conditions or with tumor cells, and they frequently result in truncated proteins or aborted translation due to the generation of a stop codon. The splice variant described here consists of a portion of intron 4 and it includes all of the coding region of exon 5 but none of exon 4, thereby coding for a polypeptide that corresponds to 71.2% of the mature native TSHβ molecule. Exon 5 of TSHβ, the coding portion retained in the novel TSHβ slice variant, is important for the biological function of TSH since it includes an 18 amino acid ‘seatbelt’ region (CNTDNSDCIHEAVRTNYC (SEQ ID NO: 4) that is used for attachment to the α-subunit. Without being bound by theory, this suggests that the splice variant may retain the ability to function as a heterodimeric complex.

The aforementioned results indicate that the novel TSHβ splice variant is preferentially expressed in the bone marrow and the thyroid, and that gene expression increases in the thyroid following systemic virus infection. Inasmuch as bone marrow cells appear to be a primary source of TSHβ in the thyroid, it is envisioned that the higher level of TSHβ gene expression is due either to an increase in TSH synthesis by resident bone marrow cells in the thyroid, or increased trafficking of bone marrow cells to the thyroid during infection.

Human Variant TSHβ

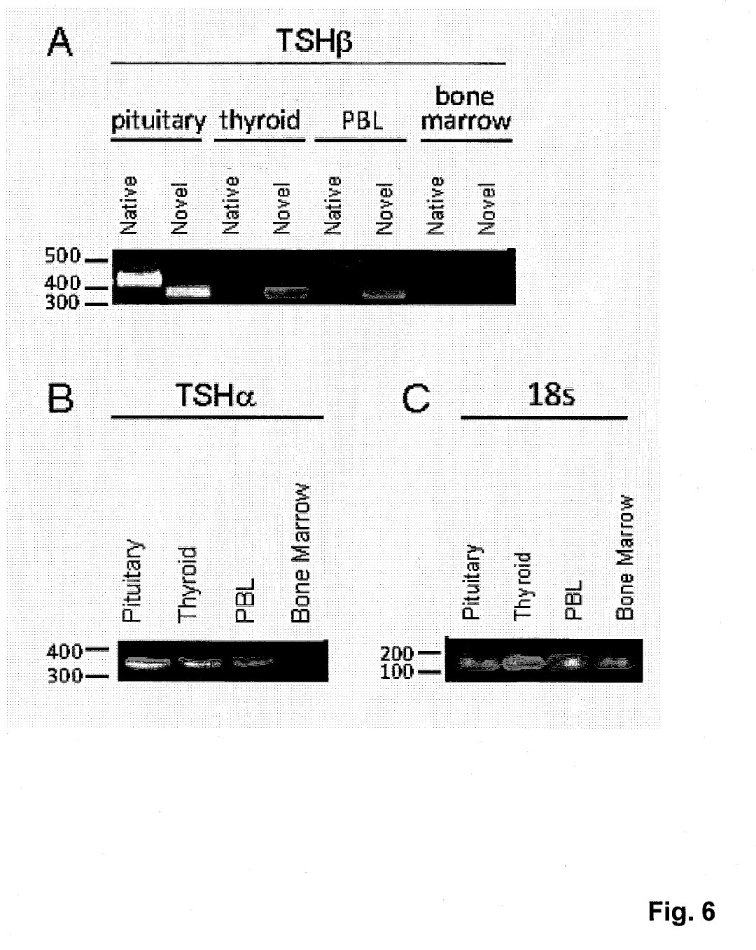

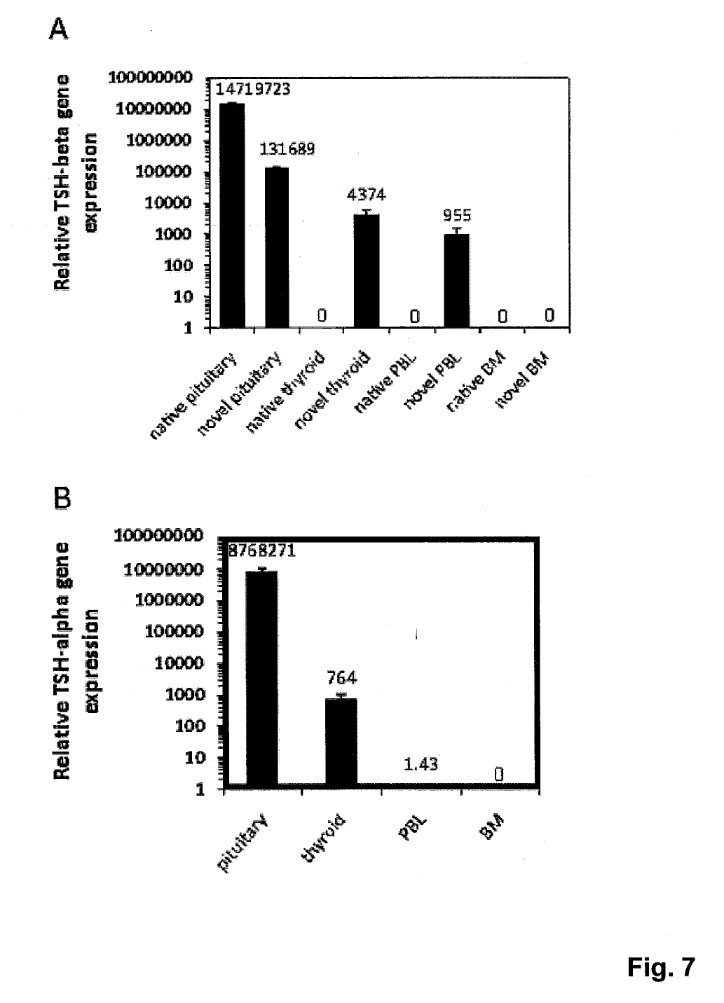

Human variant TSHβ was identified when it was discovered that human pituitary expressed a variant TSHβ isoform that is analogous to the mouse TSHβ splice variant (shown in SEQ ID NOS: 7 and 8). This novel variant consisted of a twenty-seven nucleotide portion of intron 2 and all of exon 3, coding for 71.2% of the native human TSHβ polypeptide. Of particular interest, the TSHβ splice variant was expressed at significantly higher levels than the native form or TSHβ in PBL and the thyroid. See FIGS. 6-7. The TSHα gene also was expressed in the pituitary, thyroid, and PBL, but was not detected in the bone marrow, suggesting that the TSHβ polypeptide in the thyroid and PBL may exist as a dimer with TSHα. See FIG. 7B. These findings identify a previously unknown splice variant of human TSHβ. They also have implications for immune-endocrine interactions in the thyroid, metabolic regulation during immunological stress, autoimmune thyroid disease and other TSHβ-related disorder.

It was determined that human pituitary expressed a TSHβ isoform substantially analogous to the mouse TSHβ splice variant. This novel variant consisted of a twenty-seven nucleotide portion of intron 2 and all of exon 3, coding for 71.2% of the native human TSH/3 polypeptide. Of particular interest, the TSHβ splice variant was expressed at significantly higher levels than the native form or TSHβ in PBL and the thyroid. The TSHβ gene also was expressed in the pituitary, thyroid, and PBL, but was not detected in the bone marrow, suggesting that the TSHβ polypeptide in the thyroid and PBL may exist as a dimer with TSHα.

RT-PCR amplification was done using two primer sets and RNA from human pituitary, thyroid, PBL, and bone marrow. One primer set was designed to amplify the complete human TSHβ open reading frame and consisted of an upstream primer targeted to a region in exon 2 prior to the TSHβ transcriptional start site, and a downstream primer targeted to a region in exon 3 that began one nucleotide after the stop codon. The second primer set consisted of an upstream primer targeted to a region at the end of intron 2 with the same downstream primer used for native TSHβ. Both native (SEQ ID NO: 5) and variant TSHβ (SEQ ID NO: 7) PCR products were obtained from human pituitary RNA, but only variant TSHβ was identified in human thyroid and PBL RNA. See FIGS. 6-7. Neither form of TSHβ was amplified from human bone marrow RNA. RT-PCR identified a product for TSHα from pituitary, thyroid, and PBL, but not bone marrow. See FIG. 7B. 18s gene expression was expressed at equivalent levels in all four samples. See FIG. 6C.

The human TSHβ gene consists of three exons and two introns, with portions of exons 2 and 3 coding for the TSHβ polypeptide. Analysis of intron 2 revealed a twenty-seven nucleotide sequence starting with an ATG triplet at the 3′ end that is in-frame with exon 3.

To measure the differences in human native vs. variant TSH gene expression, qRT-PCR was conducted. Gene expression values were normalized to 18s values for respective tissues RNAs using the method of Livak and Schmittgen, 2001 (Livak, K. J., Schmittgen, T. D., Analysis of relative gene expression data using real-time quantitative PCR and the 2-DDCt Method. Methods 25, 402-408, 2001). Although both native and variant human TSHβ forms were expressed in the pituitary, there was a 111-fold preference for native over novel TSHβ in the human pituitary. See FIG. 7A. That pattern was reversed in human thyroid and PBL where there was a 4,374-fold preference, and a 955-fold preference, of variant over native TSHβ gene expression in the thyroid and in PBL, respectively.

qRT-PCR analysis was done to determine if the human TSHα gene was expressed in tissues that expressed the TSHβ splice variant. The pattern of gene expression observed for the human TSHβ splice variant also was present for TSHα as seen by high level of expression in the pituitary, modest level of expression in the thyroid, low but detectable expression level in PBL, and undetectable levels of TSHα in bone marrow. See FIG. 7B.

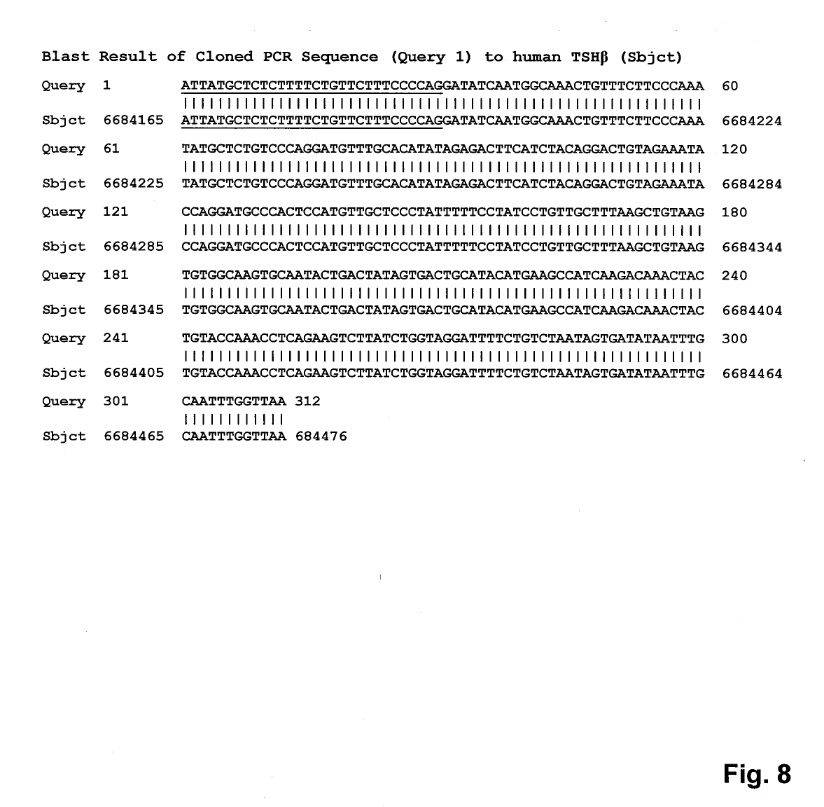

Sequence analysis of the variant human TSHβ PCR product revealed complete homology to human TSHβ (GenBank accession no. NM_{—}000549), including the twenty-seven nucleotides in intron 2 that precede exon 3. See FIG. 8. Moreover, seven of the nine amino acids coded for by human TSHβ intron nucleotides were identical to mouse TSHβ within that region. Hence, there was a high degree of organizational similarity between the human and the mouse TSHβ splice variant.

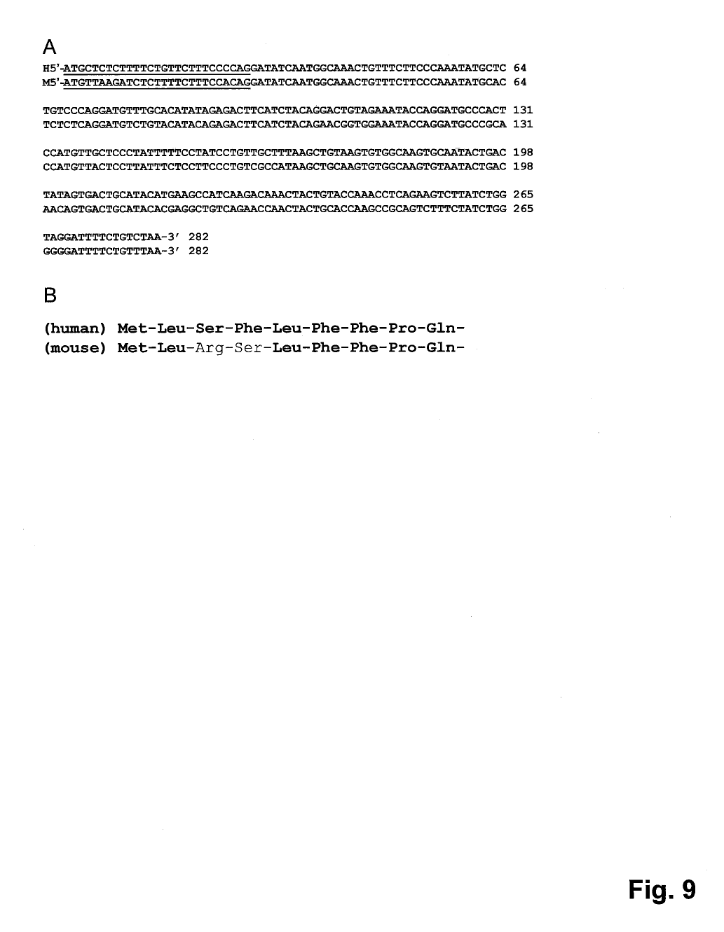

A comparison of the nucleotide sequence of the human and mouse TSHβ splice variant is shown in FIG. 9A. Within the twenty-seven nucleotide region of the putative leader sequence (FIG. 9A, underlined nucleotides), eight nucleotides differed between the two species. However, this resulted in only two amino acid substitutions, as shown in FIG. 9B at amino acid positions three and four of the leader sequence. Because those substitutions consisted of amino acids that were primarily hydrophobic or uncharged polar, a potential transmembrane function of the human leader sequence is likely. The amino acid sequence of human native TSHβ is shown in SEQ ID NO: 6 and that of human variant TSHβ is shown in SEQ ID NO: 8.

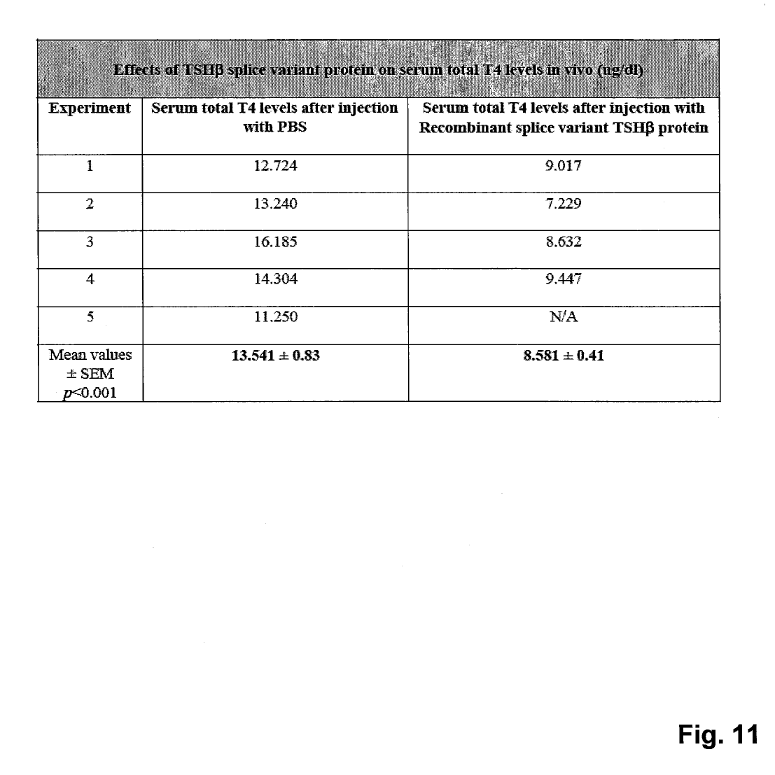

Without being bound by theory or any particular mechanism, applicants envision that there are several potential ways in which the TSH generated by the immune system might serve to regulate thyroid hormone activity. On the one hand, the immune system TSH could function agonistically to elicit a thyroid hormone response leading to increases in T3 and T4 synthesis and an upregulation in cellular and physiologic metabolic activity. Such a possibility is supported by the observation that, in the mouse, the TSHβ splice variant was capable of inducing a cAMP response. See FIG. 4. Conversely, it can also be envisioned that the TSHβ splice variant may have antagonistic activity and that it may limit thyroid hormone synthesis by binding to and competing for TSH receptor signaling. Without again being bound by theory, the inventors envision that the function of the immune system-derived TSHβ splice variant polypeptide may be to block or augment the action of pituitary-derived native TSHβ. Supporting this hypothesis is the finding that mice injected with recombinant TSHβ protein have suppressed levels of circulating total T4. See FIG. 11.

Microregulation of thyroid hormone activity by variant TSH generated by the immune system may adjust metabolic demands during times when energy conservation is needed. The immune system is especially well-suited to determine the host's energy and metabolic needs in the face of an ongoing infection or other types of immunological stress in order to conserve or re-engage energy-generating processes during and following the immune response.

The observed preferential expression of the human variant TSHβ in PBL and the thyroid supports the assertion that variant TSHβ has additional biological functions. For instance, TSHβ-producing myeloid cells can migrate to the thyroid and thus the source of intrathyroidal TSH in hematopoietic cells that have trafficked to the thyroid. Without being bound by theory or any particular mechanism, applicants envision that one possible explanation for the failure to detect variant TSHβ expression in human bone marrow is that the number of cells producing the TSHβ splice variant in the bone marrow fluctuates according to the need for those cells in the circulation. These findings are supportive of a role of immune system-derived variant TSHβ in intrathyroidal microregulation of thyroid hormone output.

In human tissue, TSHβ gene expression paralleled that observed for the TSHβ splice variant, with both being expressed in the pituitary, thyroid, and PBL, but not in the BM. See FIGS. 6-7. Gene expression of the splice variant form of TSHβ (but not native form of TSHβ) was detected in the thyroid and PBL, suggesting that the TSHβ splice variant polypeptide may pair with TSHα to form a heterodimer. This pairing could occur through the 18-amino acid ‘seatbelt’ region (CNTDNSDCIHEAVRTNYC shown in SEQ ID NO: 8), which is present in exon 3 of the human TSHβ splice variant. However, due to a lack of amino acids coded for by exon 2, the human TSHβ splice variant would lack the CAGYC peptide segment that is used to dimerize with TSHα (Hayashizaki, Y., Hiraoka, Y., Endo, Y., Miyai, K., Matsubara, K., 1989. Thyroid-stimulating hormone (TSH) deficiency caused by a single base substitution in the CAGYC region of the beta-subunit. Embo J 8, 2291-2296). Additionally, the absence of exon 2-encoded amino acids would reduce the overall glycosylation pattern of the variant TSHβ molecule, thus potentially limiting its interaction with TSHα (Szkudlinski, M. W., Fremont, V., Ronin, C., Weintraub, B. D., 2002. Thyroid-stimulating hormone and thyroid-stimulating hormone receptor structure-function relationships. Physiol Rev 82, 473-502).

These differences could have negative consequences for the host, particularly if the TSHβ splice variant displays an enhanced potential for immunogenicity relative to native TSHβ. For example, perhaps an event such as systemic virus infection, as was demonstrated in mice, triggers the production of high levels of the variant TSHβ in the thyroid, it might inadvertently lead to the generation of anti-TSH autoantibodies. Such a response could be the consequence of enhanced immunogenicity as a result of the unique physiochemical properties, altered folding, anomalous dimerization, or high local levels of variant TSHβ protein, which could in turn lead to autoimmune thyroiditis, particularly in certain genetically susceptible individuals.

Supporting this position is the finding that the human variant TSHβ is differentially expressed, as determined by quantitative realtime PCR analysis, in normal and diseased human thyroid tissues. See FIGS. 6-7. Expression of variant TSHβ was depressed, as compared to that in normal thyroid tissue, in both benign and malignant thyroid cancers as well as in thyroid tissue obtained from patients suffering Graves' disease. In contrast, thyroid tissue obtained from patients with chronic thyroiditis were determined to have elevated levels of variant TSHβ expression.

Polynucleotides

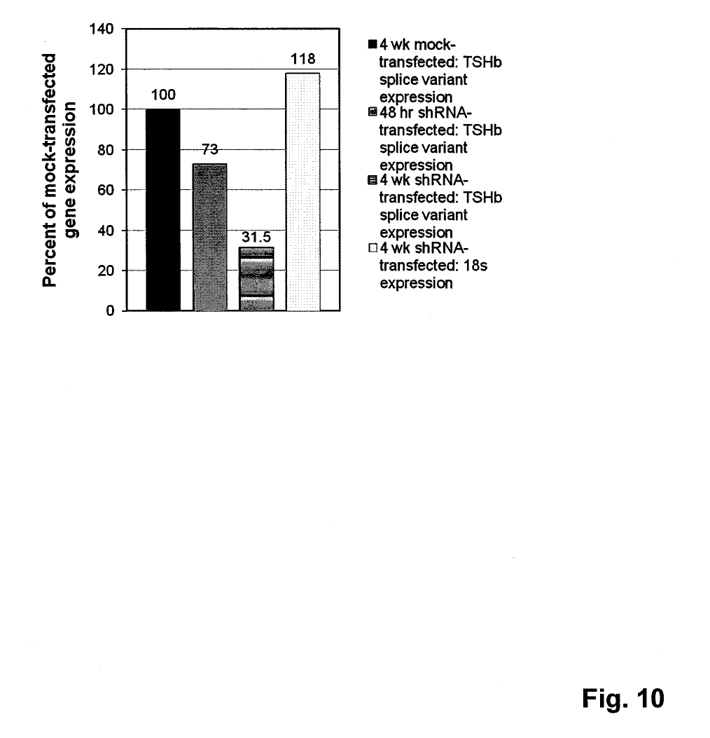

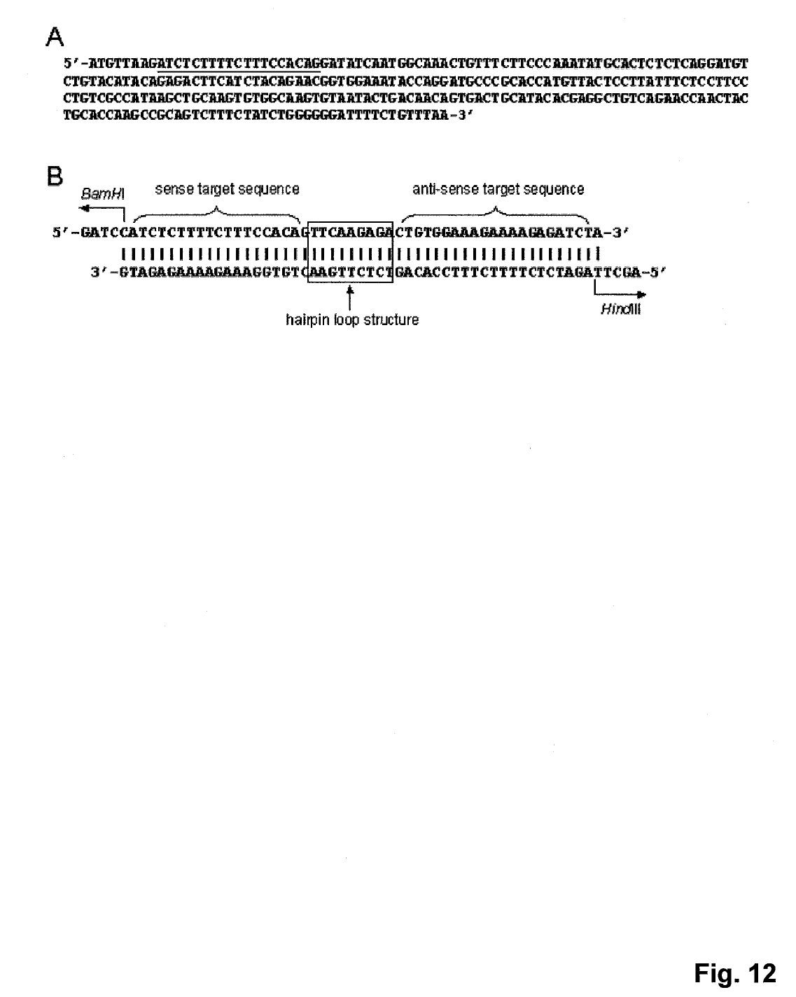

Included in the present disclosure are the nucleotides presented in the Sequence Listing, host cells expressing such nucleotides, the expression products of such nucleotides, and: (a) nucleotides that encode mammalian homologs of the described polynucleotides, including the specifically described variant TSHβ proteins, and the TSHβ protein products; (b) nucleotides that encode one or more portions of the described variant TSHβ proteins and that correspond to functional domains, and the polypeptide products specified by such nucleotide sequences, including, but not limited to, the novel regions of any active domain(s); (c) isolated nucleotides that encode mutant versions, engineered or naturally occurring, of the described variant TSHβ proteins in which all or a part of at least one domain is deleted or altered, and the polypeptide products specified by such nucleotide sequences, including, but not limited to, soluble proteins and peptides in which all or a portion of the signal (or one or more hydrophobic transmembrane) sequence is deleted; (d) nucleotides that encode chimeric fusion proteins containing all or a portion of a coding region of a TSHβ protein, or one of its domains (e.g., a receptor or ligand binding domain, accessory protein/self-association domain, etc.) fused to another peptide or polypeptide; or (e) therapeutic or diagnostic derivatives of the described polynucleotides, such as oligonucleotides, antisense polynucleotides, ribozymes, dsRNA, siRNA, or gene therapy constructs comprising a sequence first disclosed in the Sequence Listing. See, e.g., FIGS. 10 and 12 (disclosing experimental aspects of an embodiment of the present disclosure, where siRNA is used to suppress TSHβ expression).

Also included are the human DNA sequences presented in the Sequence Listing (and vectors comprising the same), and any nucleotide sequence encoding a contiguous human TSHβ protein open reading frame (ORF) that hybridizes to a complement of a DNA sequence presented in the Sequence Listing under highly stringent conditions, e.g., hybridization to filter-bound DNA in 0.5 M NaHPO₄, 7% sodium dodecyl sulfate (SDS), 1 mM EDTA at 65° C., and washing in 0.1.times.SSC/0.1% SDS at 68° C. (Ausubel et al., eds., 1989, Current Protocols in Molecular Biology, Vol. I, Green Publishing Associates, Inc., and John Wiley & Sons, Inc., N.Y., at p. 2.10.3) and encodes a functionally equivalent expression product. Additionally contemplated are any nucleotide sequences that hybridize to the complement of a DNA sequence that encodes and expresses an amino acid sequence presented in the Sequence Listing under moderately stringent conditions, e.g., washing in 0.2 times SSC/0.1% SDS at 42° C. (Ausubel et al., 1989, supra), yet still encodes a functionally equivalent TSHβ product. Functional equivalents of a TSHβ protein include naturally occurring variant TSHβ proteins present in other species, and mutant variant TSHβ proteins, whether naturally occurring or engineered (e.g., by site directed mutagenesis, gene shuffling, directed evolution as described in, for example, U.S. Pat. No. 5,837,458). The invention also includes degenerate nucleic acid variants of the disclosed TSHβ polynucleotide sequences.

Additionally contemplated are polynucleotides encoding TSHβ ORFs, or their functional equivalents, encoded by polynucleotide sequences that are about 99, 95, 90, or about 85 percent similar or identical to corresponding regions of the nucleotide sequences of the Sequence Listing (as measured by BLAST sequence comparison analysis using, for example, the GCG sequence analysis package, as described herein, using standard default settings).

Applications of Polynucleotides

Some applications include nucleic acid molecules, preferably DNA molecules, that hybridize to, and are therefore the complements of, the described TSHβ nucleotide sequences or alternate splice variants thereof. Such hybridization conditions may be highly stringent or less highly stringent, as described herein. In instances where the nucleic acid molecules are deoxyoligonucleotides (“DNA oligos”), such molecules are generally about 16 to about 100 bases long, or about 20 to about 80 bases long, or about 34 to about 45 bases long, or any variation or combination of sizes represented therein that incorporate a contiguous region of sequence first disclosed in the Sequence Listing. Such oligonucleotides can be used in conjunction with the polymerase chain reaction (PCR) to screen libraries, isolate clones, and prepare cloning and sequencing templates, etc.

Alternatively, such TSHβ oligonucleotides can be used as hybridization probes for screening libraries, and assessing gene expression patterns (particularly using a microarray or high-throughput “chip” format). Additionally, a series of TSHβ oligonucleotide sequences, or the complements thereof, can be used to represent all or a portion of the described TSHβ sequences. An oligonucleotide or polynucleotide sequence first disclosed in at least a portion of one or more of the sequences of SEQ ID NOS: 1, 3, 5 and 7 can be used as a hybridization probe in conjunction with a solid support matrix/substrate (resins, beads, membranes, plastics, polymers, metal or metallized substrates, crystalline or polycrystalline substrates, etc.). Of particular note are spatially addressable arrays (i.e., gene chips, microtiter plates, etc.) of oligonucleotides and polynucleotides, or corresponding oligopeptides and polypeptides, wherein at least one of the biopolymers present on the spatially addressable array comprises an oligonucleotide or polynucleotide sequence first disclosed in at least one of the sequences of SEQ ID NOS: 1, 3, 5 and 7, or an amino acid sequence encoded thereby. Methods for attaching biopolymers to, or synthesizing biopolymers on, solid support matrices, and conducting binding studies thereon, are disclosed in, inter alia, U.S. Pat. Nos. 5,700,637, 5,556,752, 5,744,305, 4,631,211, 5,445,934, 5,252,743, 4,713,326, 5,424,186, and 4,689,405, the disclosures of which are herein incorporated by reference in their entirety.

Addressable arrays comprising sequences first disclosed in SEQ ID NOS: 1-8 can be used to identify and characterize the temporal and tissue specific expression of a gene. These addressable arrays incorporate oligonucleotide sequences of sufficient length to confer the required specificity, yet they must be within the limitations of the production technology. The length of these probes is usually within a range of between about 8 to about 2000 nucleotides. Preferably, the probes consist of 60 nucleotides, and more preferably 25 nucleotides, from the sequences shown in SEQ ID NOS: 1, 3, 5 and 7.

For example, a series of the described TSHβ oligonucleotide sequences, or the complements thereof, can be used in chip format to represent all or a portion of the described sequences. The oligonucleotides, typically between about 16 to about 40 (or any whole number within the stated range) nucleotides in length, can partially overlap each other, and/or the sequence may be represented using oligonucleotides that do not overlap. Accordingly, the described polynucleotide sequences shall typically comprise at least about two or three distinct oligonucleotide sequences of at least about 8 nucleotides in length that are each first disclosed in the described Sequence Listing. Such oligonucleotide sequences can begin at any nucleotide present within a sequence in the Sequence Listing, and proceed in either a sense (5′-to-3′) orientation vis-a-vis the described sequence or in an antisense orientation.

Microarray-based analysis allows the discovery of broad patterns of genetic activity, providing new understanding of gene functions, and generating novel and unexpected insight into transcriptional processes and biological mechanisms. The use of addressable arrays comprising sequences shown in SEQ ID NOS: 1-8 provides detailed information about transcriptional changes involved in a specific pathway, potentially leading to the identification of novel components, or gene functions that manifest themselves as novel phenotypes.

Probes consisting of sequences shown in SEQ ID NOS: 1-8 can also be used in the identification, selection, and validation of novel molecular targets for drug discovery. The use of these unique sequences permits the direct confirmation of drug targets, and recognition of drug dependent changes in gene expression that are modulated through pathways distinct from the intended target of the drug. These unique sequences therefore also have utility in defining and monitoring both drug action and toxicity.

As an example of utility, the sequences shown in SEQ ID NOS: 1-8 can be utilized in microarrays, or other assay formats, to screen collections of genetic material from patients who have a particular medical condition. These investigations can also be carried out using the sequences shown in SEQ ID NOS: 1-8 in silico and by comparing previously collected genetic databases and the disclosed sequences using computer software known to those in the art.

Thus, the sequences first disclosed in SEQ ID NOS: 1-8 can be used to identify mutations associated with a particular TSHβ-related disorder.

Therapeutic Applications

TSH preparations (for example, but not limited to, cells expressing TSHβ, TSHβ proteins, peptides, alternative splice variants or fragments thereof) or TSHβ-antagonists (e.g., antibodies, other molecules that interfere with TSHβ's activity or molecules that retard or inhibit the functional expression of TSHβ such as TSHβ antisense or small inhibitory RNA molecules) present opportunities for therapeutic intervention in treating a wide variety of conditions that have been linked to TSHβ.

Diseases and disorders associated with human variant TSHβ proteins include, but are not limited to, diseases associated with TSH, such as TSHβ-related disorders, hypothyroidism (underactive thyroid), hyperthyroidism (overactive thyroid), autoimmune thyroid diseases, Graves' disease and Hashimoto's thyroiditis, Graves' Ophthalmopathy, thyroid nodules, Pendred's Syndrome, post-traumatic stress disorder, chronic diseases such as Lyme disease which can result in TSHβ-related disorders, osteoporosis, obesity, infertility, autoimmune and inflammatory disorders including, but not limited to, acute and chronic inflammation, inflammation associated with rheumatic disorders such as rheumatoid arthritis, system lupus erythematosus, a vasculitic disorder, or another rheumatic disorder, allergic responses, psoriasis, or dermatitis; inflammation in the respiratory tract, lung, or sinus associated with asthma, chronic obstructive pulmonary disease, chronic bronchitis, or emphysema; inflammation located in the gastrointestinal tract, Inflammatory Bowel disease, ulcerative colitis, Crohn's disease or diarrhea; inflammation associated with single-organ or multi-organ failure or sepsis; and inflammation associated with chronic active hepatitis, alcoholic liver disease, or non-alcoholic fatty liver disease. (collectively TSHβ-related disorders). Accordingly, the described novel splice variants of human TSHβ protein can be useful in detecting and treating such conditions, for example by using TSH containing variant TSHβ as an anti-inflammatory agent in a broad spectrum of inflammatory conditions, including methods using variant TSHβ to potentiate the effect of glucocorticoid treatment. Therefore, provided is a method for treating inflammation, comprising administering a therapeutically sufficient amount of variant TSH polypeptide to a mammal, wherein administration of the polypeptide results in a clinically significant improvement in the inflammatory condition of the mammal.

In some applications the described variant human TSHβ is targeted (by drugs, oligos, antibodies, etc.) in order to treat disease, or to therapeutically augment the efficacy of, for example, chemotherapeutic agents used in the treatment of TSHβ-related disorders.

In some cases the use of small molecule inhibitors of TSHβ expression and/or activity and large molecules to effect the level, activity, or bioavailability of TSHβ in vivo, including, but not limited to, mutant TSHβ proteins or peptides that compete with native TSHβ, anti-TSHβ antibodies, and nucleotide sequences that can be used to inhibit (reduce or eliminate) TSHβ gene expression (including, but not limited to, small interfering RNA (siRNA), small hairpin RNA (shRNA), antisense, ribozyme, and/or triplex molecules, and coding or regulatory sequence replacement constructs). See, e.g., FIGS. 10 and 12 (disclosing experimental aspects of an embodiment of the present disclosure, where siRNA is used to suppress TSHβ expression). In certain embodiments of the present invention, such compounds, or pharmaceutical compositions comprising one or more such compounds, can be used as prophylactic or therapeutic agents for the prevention or treatment of TSHβ-related disorders, or any of a wide variety of symptoms or conditions associated with TSHβ-related disorders.

In other cases, one or more such compounds are used to treat or prevent TSHβ-related disorders or related diseases, disorders, or conditions. Such compounds are manufactured of a medicament for treating or preventing TSHβ-related disorders is also contemplated. Compositions comprising a biologically or therapeutically effective amount of one or more of such compounds for use in the preparation of a medicament for use in prevention and/or treatment of TSHβ-related disorders.

The use of antagonists of TSHβ (including small molecules and large molecules), mutant versions of TSHβ, or portions thereof, that compete with native TSHβ, TSHβ antibodies, as well as nucleotide sequences that can be used to inhibit expression of TSHβ (e.g., antisense, siRNA, triplex, and ribozyme molecules, and gene or regulatory sequence replacement constructs), in the treatment of TSHβ-related disorders, such as, but not limited to, hypothyroidism, hyperthyroidism, autoimmune thyroid diseases, Graves' disease and Hashimoto's thyroiditis. Compounds including, but not limited to, those identified via assay techniques such as those described herein, are tested for the ability to ameliorate symptoms associated with TSHβ-related disorders such as hypothyroidism, hyperthyroidism, autoimmune thyroid diseases, Graves' disease and Hashimoto's thyroiditis and related diseases and disorders.

Assays

The assays described herein can identify compounds that affect TSHβ activity or TSHβ gene activity (by affecting TSHβ gene expression, including molecules, e.g., proteins or small organic molecules, that affect or interfere with splicing events so expression of full-length or a truncated form of TSHβ can be modulated). However, it should be noted that such assays can also be used to identify compounds that indirectly modulate TSHβ. The identification and use of compounds that affect a TSHβ-independent step in a TSHβ pathway are also within the scope of the invention. Compounds that indirectly affect TSHβ activity can also be used in therapeutic methods for the treatment of TSHβ-related disorders, such as hypothyroidism, hyperthyroidism, autoimmune thyroid diseases, Graves' disease and Hashimoto's thyroiditis.

In some cases, cell-based and animal model-based assays are used for the identification of compounds exhibiting an ability to ameliorate the symptoms of TSHβ related disorders, such as hypothyroidism, hyperthyroidism, autoimmune thyroid diseases, Graves' disease and Hashimoto's thyroiditis. Cell-based systems used to identify compounds that may act to ameliorate TSHβ-related disorder symptoms include, for example, recombinant or non-recombinant cells, such as cell lines that express a TSHβ sequence. Host cells (e.g., COS cells, CHO cells, fibroblasts) genetically engineered to express a functional TSHβ can also be used.

In utilizing such cell systems, cells are exposed to a compound suspected of exhibiting an ability to ameliorate the symptoms of a TSHβ-related disorder, such as hypothyroidism, hyperthyroidism, autoimmune thyroid diseases, Graves' disease and Hashimoto's thyroiditis, at a concentration and for a time sufficient to elicit such an amelioration of the SHβ-related disorder, such as, hypothyroidism, hyperthyroidism, autoimmune thyroid diseases, Graves' disease and Hashimoto's thyroiditis. After exposure, the cells are assayed to measure alterations in TSHβ expression, e.g., by assaying cell lysates for TSHβ mRNA transcripts (e.g., by Northern analysis or RT-PCR), or by assaying for the level of TSHβ protein expressed in the cell (e.g., by SDS-PAGE and Western blot or immunoprecipitation). Compounds that reduce TSHβ expression or activity are good candidates as therapeutics. Alternatively, the cells are examined to determine whether one or more TSHβ-related disorder like phenotype has been altered to resemble a more normal or TSHβ-related disorder-like phenotype, or a phenotype more likely to produce a lower incidence or severity of a TSHβ-related disorder. Expression and/or activity of components of a signal transduction pathway of which TSHβ and TSH is a part, or a TSHβ signal transduction pathway itself, can also be assayed.

In some cases, animal-based model systems can be used to identify compounds capable of preventing, treating, or ameliorating symptoms associated with TSHβ-related disorders such as hypothyroidism, hyperthyroidism, autoimmune thyroid diseases, Graves' disease and Hashimoto's thyroiditis. These animals may be transgenic, knock-out, or knock-in animals (preferably humanized knock-in animals where, for example, the endogenous animal TSHβ gene has been replaced by a human TSHβ sequence), as described herein.

Knock-out mice can be produced in several ways, one of which involves the use of mouse embryonic stem cell (“ES cell”) lines that contain gene trap mutations in a murine homolog of at least one of the described human transporter sequences. When the unique TSHβ protein sequences described are “knocked-out”, they provide a method of identifying phenotypic expression of the particular gene, as well as a method of assigning function to previously unknown genes. In addition, animals in which the TSHβ sequences described in SEQ ID NOS: 1, 3, 5 and 7 are “knocked-out” provide a unique source in which to elicit antibodies to homologous and orthologous proteins, which would have been previously viewed by the immune system as “self” and therefore would have failed to elicit significant antibody responses. To these ends, gene trapped knockout ES cells have been generated using murine homologs of certain of the described variant TSHβ proteins.

Such animal models are used as test substrates for identification of drugs, pharmaceuticals, therapies, and interventions that are effective in preventing or treating TSHβ-related disorders such as hypothyroidism, hyperthyroidism, autoimmune thyroid diseases, Graves' disease and Hashimoto's thyroiditis. For example, animal models are exposed to a compound suspected of exhibiting an ability to modulate TSHβ-related disorders such as hypothyroidism, hyperthyroidism, autoimmune thyroid diseases, Graves' disease and Hashimoto's thyroiditis, at a sufficient concentration and for a time sufficient to elicit such an amelioration of TSHβ-related disorders in the exposed animals. The response of the animals to the exposure are monitored by assessing the reversal of symptoms associated with TSHβ-related disorders such as hypothyroidism, hyperthyroidism, autoimmune thyroid diseases, Graves' disease and Hashimoto's thyroiditis. Any treatments that prevent, reverse, halt, or slow the progression of any aspect of symptoms associated with a TSHβ-related disorder should be considered as candidates for therapeutic intervention in the prevention or treatment of TSHβ-related disorder such as hypothyroidism, hyperthyroidism, autoimmune thyroid diseases, Graves' disease and Hashimoto's thyroiditis. Dosages of test agents are determined by deriving toxicity and dose-response curves.

In particular aspects of the present invention, one or more compounds of the present invention is administered in combination with one or more additional compounds or drugs (“additional active agents”) for the treatment, management, and/or prevention of TSHβ-related disorders such as hypothyroidism, hyperthyroidism, autoimmune thyroid diseases, Graves' disease and Hashimoto's thyroiditis.

Toxicity and therapeutic efficacy of such compounds are determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, expressed as the ratio LD₅₀/ED₅₀. Compounds that exhibit large therapeutic indices are preferred. Compounds that exhibit toxic side effects are used in certain embodiments, however, care should usually be taken to design delivery systems that target such compounds preferentially to the site of affected tissue, in order to minimize potential damage to uninfected cells and, thereby, reduce side effects.

Data obtained from cell culture assays and animal studies are used in formulating a range of dosages for use in humans. In some cases it is preferred that the dosages of such compounds Ile within a range of circulating concentrations that include the ED₅₀ with little or no toxicity. The dosage may vary within this range depending on the dosage form employed and the route of administration utilized. For any compound used according to the applications described, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information is used to more accurately determine useful doses in humans. Plasma levels may be measured, for example, by high performance liquid chromatography.

Therapeutic Applications and Compositions

When the therapeutic treatment of TSHβ-related disorders such as hypothyroidism, hyperthyroidism, autoimmune thyroid diseases, Graves' disease and Hashimoto's thyroiditis, is contemplated, the appropriate dosage is determined using animal studies to determine the maximal tolerable dose, or MTD, of a bioactive agent per kilogram weight of the test subject. In general, at least one animal species tested is mammalian. Those skilled in the art regularly extrapolate doses for efficacy and avoiding toxicity to other species, including human. Before human studies of efficacy are undertaken, Phase I clinical studies help establish safe doses.

Additionally, if deemed necessary, the bioactive agent is complexed with a variety of well established compounds or structures that, for instance, enhance the stability of the bioactive agent, or otherwise enhance its pharmacological properties (e.g., increase in vivo half-life, reduce toxicity, etc.).

The therapeutic agents can be administered by any number of methods known to those of ordinary skill in the art including, but not limited to, oral administration, inhalation, subcutaneous (sub-q), intravenous (I.V.), intraperitoneal (I.P.), intramuscular (I.M.), or intrathecal injection, or topically applied (transderm, ointments, creams, salves, eye drops, and the like), as described in greater detail below.

Pharmaceutical compositions used in the applications described are formulated in conventional manners using one or more physiologically acceptable carriers or excipients. The pharmaceutical compositions can comprise formulation materials for modifying, maintaining, or preserving, for example, the pH, osmolarity, viscosity, clarity, color, isotonicity, odor, sterility, stability, rate of dissolution or release, adsorption or penetration of the composition. Suitable formulation materials include, but are not limited to: amino acids (for example, glycine, glutamine, asparagine, arginine and lysine); antimicrobials; antioxidants (for example, ascorbic acid, sodium sulfite and sodium hydrogen-sulfite); buffers (for example, borate, bicarbonate, Tris-HCl, citrates, phosphates and other organic acids); bulking agents (for example, mannitol and glycine); chelating agents (for example, ethylenediamine tetraacetic acid (EDTA)); complexing agents (for example, caffeine, polyvinylpyrrolidone, beta-cyclodextrin, and hydroxypropyl-beta-cyclodextrin); fillers; monosaccharides, disaccharides, and other carbohydrates (for example, glucose, mannose and dextrins); proteins (for example, serum albumin, gelatin and immunoglobulins); coloring, flavoring, and diluting agents; emulsifying agents; hydrophilic polymers (for example, polyvinylpyrrolidone); low molecular weight polypeptides; salt-forming counterions (for example, sodium); preservatives (for example, benzalkonium chloride, benzoic acid, salicylic acid, thimerosal, phenethyl alcohol, methylparaben, propylparaben, chlorhexidine, sorbic acid and hydrogen peroxide); solvents (for example, glycerin, propylene glycol and polyethylene glycol); sugar alcohols (for example, mannitol and sorbitol); suspending agents; surfactants or wetting agents (for example, pluronics, PEG, sorbitan esters, polysorbates (for example, polysorbate 20 and polysorbate 80), triton, tromethamine, lecithin, cholesterol, and tyloxapal); stability enhancing agents (for example, sucrose and sorbitol); tonicity enhancing agents (for example, alkali metal halides (for example, sodium or potassium chloride), mannitol, and sorbitol); delivery vehicles; diluents; excipients; and pharmaceutical adjuvants (“Remington's Pharmaceutical Sciences”, 18^{th} Ed. (Gennaro, ed., Mack Publishing Company, Easton, Pa., 1990)).

Additionally, an antibody to TSHβ, variant TSHβ, or other therapeutic molecule can be linked to a half-life extending vehicle. Certain exemplary half-life extending vehicles are known in the art, and include, but are not limited to, the Fc domain, polyethylene glycol, and dextran (see, e.g., PCT Patent Application Publication No. WO 99/25044).

The compounds and their physiologically acceptable salts and solvates may be formulated for administration by inhalation or insufflation (either through the mouth or the nose), or oral, buccal, parenteral or rectal administration. For oral administration, the pharmaceutical compositions sometimes take the form of, for example, tablets or capsules prepared by conventional means with pharmaceutically acceptable excipients such as binding agents (e.g., pregelatinised maize starch, polyvinylpyrrolidone or hydroxypropyl methylcellulose), fillers (e.g., lactose, microcrystalline cellulose or calcium hydrogen phosphate), lubricants (e.g., magnesium stearate, talc or silica), disintegrants (e.g., potato starch or sodium starch glycolate), or wetting agents (e.g., sodium lauryl sulphate). The tablets are coated by methods well-known in the art. Liquid preparations for oral administration may take the form of, for example, solutions, syrups or suspensions, or are presented as a dry product for constitution with water or other suitable vehicle before use. Such liquid preparations are prepared by conventional means with pharmaceutically acceptable additives such as suspending agents (e.g., sorbitol syrup, cellulose derivatives or hydrogenated edible fats), emulsifying agents (e.g., lecithin or acacia), non-aqueous vehicles (e.g., almond oil, oily esters, ethyl alcohol or fractionated vegetable oils), and preservatives (e.g., methyl or propyl-p-hydroxybenzoates or sorbic acid). In some cases, the preparations contain buffer salts, flavoring agents, coloring agents and sweetening agents as appropriate. Preparations for oral administration may also be suitably formulated to give controlled release of the active compound. For buccal administration the compositions may take the form of tablets or lozenges formulated in conventional manner.

For administration by inhalation, the compounds are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges of, e.g., gelatin, for use in an inhaler or insufflator are formulated containing a powder mix of the compound and a suitable powder base such as lactose or starch.

In other cases, compounds (for example variant TSHβ or inhibitors of variant TSHβ) are formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated as compositions for rectal administration such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. For example, compounds may be formulated with suitable polymeric or hydrophobic materials (for example as an emulsion in an acceptable oil), ion exchange resins, or as sparingly soluble derivatives, for example, as a sparingly soluble salt. The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration.

Active ingredients of the invention can be administered by controlled release means or by delivery devices that are well-known to those of ordinary skill in the art. Examples include, but are not limited to, those described in U.S. Pat. Nos. 3,845,770, 3,916,899, 3,536,809, 3,598,123, 4,008,719, 5,674,533, 5,059,595, 5,591,767, 5,120,548, 5,073,543, 5,639,476, 5,354,556, and 5,733,566. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof, to provide the desired release profile in varying proportions. Exemplary sustained release matrices include, but are not limited to, polyesters, hydrogels, polylactides (see, e.g., U.S. Pat. No. 3,773,919 and European Patent Application Publication No. EP 058,481), copolymers of L-glutamic acid and gamma ethyl-L-glutamate (see, e.g., Sidman et al., Biopolymers 22:547-556, 1983), poly (2-hydroxyethyl-methacrylate) (see, e.g., Langer et al., J. Biomed. Mater. Res. 15:167-277, 1981, and Langer, Chemtech 12:98-105, 1982), ethylene vinyl acetate (Langer et al., supra), and poly-D(−)-3-hydroxybutyric acid (European Patent Application Publication No. EP 133,988). Sustained release compositions may include liposomes, which can be prepared by any of several methods known in the art (see, e.g., Eppstein et al., Proc. Natl. Acad. Sci. USA 82:3688-3692, 1985, and European Patent Application Publication Nos. EP 036,676, EP 088,046, and EP 143,949). Suitable controlled-release formulations known to those of ordinary skill in the art, including those described herein, can be readily selected for use with the compounds of this invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this relatively constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

Kits

In some cases, active ingredients of the invention are preferably not administered to a patient at the same time or by the same route of administration. This invention therefore encompasses kits that, when used by the medical practitioner, can simplify the administration of appropriate amounts of active ingredients to a patient. One example to such a kit includes an ELISA to determine the level of variant TSHβ in a patient.

A typical kit comprises a single unit dosage form of one or more of the compounds of this invention, or a pharmaceutically acceptable salt, prodrug, solvate, hydrate, or stereoisomer thereof, and a single unit dosage form of another agent that may be used in combination with the compounds of this invention. Kits of the invention can further comprise devices that are used to administer the active ingredients. Examples of such devices include, but are not limited to, syringes, drip bags, patches, and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate. However, in specific embodiments, the formulations of the invention do not contain any alcohols or other co-solvents, oils or proteins. Although the presently described sequences have been specifically described using nucleotide sequence, it should be appreciated that each of the sequences can uniquely be described using any of a wide variety of additional structural attributes, or combinations thereof.

For example, a given sequence is described by the net composition of the nucleotides present within a given region of the sequence, in conjunction with the presence of one or more specific oligonucleotide sequence(s) shown in SEQ ID NOS: 1, 3, 5 and 7. Alternatively, a restriction map specifying the relative positions of restriction endonuclease digestion sites, or various palindromic or other specific oligonucleotide sequences, is used to structurally describe a given sequence. Such restriction maps, which are typically generated by widely available computer programs (e.g., the University of Wisconsin GCG sequence analysis package, SEQUENCHER 3.0, Gene Codes Corp., Ann Arbor, Mich., etc.), are optionally used in conjunction with one or more discrete nucleotide sequence(s) present in the sequence that is described by the relative position of the sequence relative to one or more additional sequence(s) or one or more restriction sites present in the disclosed sequence.

Oligonucleotide Probes

For oligonucleotide probes, highly stringent conditions may refer, e.g., to washing in 6 times SSC/0.05% sodium pyrophosphate at 37° C. (for 14-base oligos) 48° C. (for 17-base oligos), 55° C. (for 20-base oligos), and 60° C. (for 23-base oligos). These nucleic acid molecules may encode or act as TSHβ antisense molecules useful, for example, in TSHβ protein gene regulation and/or as antisense primers in amplification reactions of TSHβ nucleic acid sequences. With respect to TSHβ protein gene regulation, such techniques are used to regulate biological functions. Further, such sequences may be used as part of ribozyme and/or triple helix sequences that are also useful for TSHβ protein gene regulation.

Inhibitory antisense or double stranded oligonucleotides may additionally comprise at least one modified base moiety that is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, β-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N-6-adenine, 7-methylguanine) 5-methylaminomethyluracil, 5-methoxyaminomethyl-2-thiouracil, β-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. The antisense oligonucleotide can also comprise at least one modified sugar moiety selected from the group including, but not limited to, arabinose, 2-fluoroarabinose, xylulose, and hexose. Antisense oligonucleotides comprise at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

The antisense oligonucleotide may include an α-anomeric oligonucleotide which form specific double-stranded hybrids with complementary RNA in which, contrary to the usual (3-units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330). Alternatively, double stranded RNA is used to disrupt the expression and function of a targeted TSHβ. Such oligonucleotides are synthesized by standard methods known in the art, e.g., by use of an automated DNA synthesizer (such as are commercially available from Biosearch, Applied Biosystems, etc.). As examples, phosphorothioate oligonucleotides can be synthesized by the method of Stein et al. (1988, Nucl. Acids Res. 16:3209), and methylphosphonate oligonucleotides can be prepared by use of controlled pore glass polymer supports (Sarin et al., 1988, Proc. Natl. Acad. Sci. USA 85:7448-7451), etc.

Low stringency conditions are well-known to those of skill in the art, and will vary predictably depending on the specific organisms from which the library and the labeled sequences are derived. For guidance regarding such conditions, see, for example, Sambrook et al., 1989, Molecular Cloning, A Laboratory Manual, Cold Spring Harbor Press, Cold Spring Harbor, N.Y. (and periodic updates thereof), and Ausubel et al., 1989, supra.

In some applications suitably labeled TSHβ nucleotide probes are used to screen a human genomic library using appropriately stringent conditions or by PCR. The identification and characterization of human genomic clones is helpful for identifying polymorphisms (including, but not limited to, nucleotide repeats, microsatellite alleles, single nucleotide polymorphisms, or coding single nucleotide polymorphisms), determining the genomic structure of a given locus/allele, and designing diagnostic tests. For example, sequences derived from regions adjacent to the intron/exon boundaries of the human gene can be used to design primers for use in amplification assays to detect mutations within the exons, introns, splice sites (e.g., splice acceptor and/or donor sites), etc., that are used in diagnostics and pharmacogenomics.

For example, in some applications, the present sequences are used in restriction fragment length polymorphism (RFLP) analysis to identify specific individuals. In this technique, an individual's genomic DNA is digested with one or more restriction enzymes, and probed on a Southern blot to yield unique bands for identification (as generally described in U.S. Pat. No. 5,272,057, incorporated herein by reference). In other applications, the sequences are used to provide polynucleotide reagents, e.g., PCR primers, targeted to specific loci in the human genome, which can enhance the reliability of DNA-based forensic identifications by, for example, providing another “identification marker” (i.e., another DNA sequence that is unique to a particular individual). Actual base sequence information is used for identification as an accurate alternative to patterns formed by restriction enzyme generated fragments.

Isolation and Uses of TSHβ Genes and Nucleic Acids

A TSHβ protein gene homolog is isolated from nucleic acid from an organism of interest by performing PCR using two degenerate or “wobble” oligonucleotide primer pools designed on the basis of amino acid sequences within the TSHβ protein products disclosed herein. The template for the reaction may be genomic DNA, or total RNA, mRNA, and/or cDNA obtained by reverse transcription of mRNA prepared from human or non-human cell lines or tissue known to express, or suspected of expressing, an allele of a TSHβ protein gene.

The PCR product is subcloned and sequenced to ensure that the amplified sequences represent the sequence of the desired TSHβ protein gene. The PCR fragment is then used to isolate a full length cDNA clone by a variety of methods. For example, the amplified fragment is labeled and used to screen a cDNA library, such as a bacteriophage cDNA library. Alternatively, the labeled fragment is used to isolate genomic clones via the screening of a genomic library.

PCR technology can also be used to isolate full length cDNA sequences. For example, RNA is isolated, following standard procedures, from an appropriate cellular or tissue source (i.e., one known to express, or suspected of expressing, a TSHβ protein gene). A reverse transcription (RT) reaction is then performed on the RNA using an oligonucleotide primer specific for the most 5′ end of the amplified fragment for the priming of first strand synthesis. The resulting RNA/DNA hybrid is “tailed” using a standard terminal transferase reaction, the hybrid is digested with RNase H, and second strand synthesis is primed with a complementary primer. Thus, cDNA sequences upstream of the amplified fragment are isolated. For a review of cloning strategies that can be used, see, e.g., Sambrook et al., 1989, supra.

Alternatively, cDNA encoding a mutant TSH/3 protein sequence is isolated, for example, by using PCR. In this case, the first cDNA strand may be synthesized by hybridizing an oligo-dT oligonucleotide to mRNA isolated from tissue known to express, or suspected of expressing, a TSHβ protein, in an individual putatively carrying a mutant TSHβ protein allele, and by extending the new strand with reverse transcriptase. The second strand of the cDNA is then synthesized using an oligonucleotide that hybridizes specifically to the 5′ end of the normal sequence. Using these two primers, the product is then amplified via PCR, optionally cloned into a suitable vector, and subjected to DNA sequence analysis through methods well-known to those of skill in the art. By comparing the DNA sequence of the mutant TSHβ protein allele to that of a corresponding normal TSHβ protein allele, the mutation(s) responsible for the loss or alteration of function of the mutant TSHβ protein gene product can be ascertained.

A genomic library can also be constructed using DNA obtained from an individual suspected of carrying, or known to carry, a mutant TSHβ protein allele (e.g., a person manifesting a TSHβ protein-associated phenotype such as, for example, thyroid disorders such as hypothyroidism, hyperthyroidism, autoimmune thyroid diseases, Graves' disease and Hashimoto's thyroiditis, etc.), or a cDNA library is constructed using RNA from a tissue known to express, or suspected of expressing, a mutant TSHβ protein allele. A normal TSHβ protein gene, or any suitable fragment thereof, is labeled and used as a probe to identify the corresponding mutant TSHβ protein allele in such libraries. Clones containing mutant TSHβ sequences are purified and subjected to sequence analysis according to methods well-known to those skilled in the art. Alternatively, an expression library is constructed utilizing cDNA synthesized from, for example, RNA isolated from a tissue known to express, or suspected of expressing, a mutant TSHβ protein allele in an individual suspected of carrying, or known to carry, such a mutant allele. In this manner, gene products made by the putatively mutant tissue can be expressed and screened using standard antibody screening techniques in conjunction with antibodies raised against a normal TSHβ protein product, as described below (for screening techniques, see, for example, Harlow and Lane, eds., 1988, “Antibodies: A Laboratory Manual”, Cold Spring Harbor Press, Cold Spring Harbor, N.Y.).

The products of the described libraries are screened with labeled TSHβ protein fusion proteins, such as, for example, alkaline phosphatase-TSHβ protein or TSHβ protein-alkaline phosphatase fusion proteins. In cases where a TSHβ protein mutation results in an expression product with altered function (e.g., as a result of a missense or a frameshift mutation), polyclonal antibodies to a TSHβ protein are likely to cross-react with a corresponding mutant TSHβ protein expression product. Library clones detected via their reaction with such labeled antibodies can be purified and subjected to sequence analysis according to methods well-known in the art. Included, therefore is the use of nucleotide sequences that encode mutant isoforms of any of the TSHβ amino acid sequences, peptide fragments thereof, truncated versions thereof, and/or fusion proteins including any of the above fused to another unrelated polypeptide. Examples of such polypeptides include, but are not limited to, an epitope tag that aids in purification or detection of the resulting fusion protein, or an enzyme, fluorescent protein, or luminescent protein that is used as a marker.

In some embodiments, TSHβ nucleic acid molecules encode or act as antisense molecules, useful, for example, in TSHβ gene regulation, and/or as antisense primers in amplification reactions of TSHβ nucleic acid sequences. See, e.g., FIGS. 10 and 12. With respect to TSHβ gene regulation, such methods are used to regulate one or more of the biological functions associated with TSHβ, as described herein. Further, such sequences are used as part of ribozyme and/or triple helix sequences that are also useful for TSHβ gene regulation. Such antisense nucleic acids encompass an RNA molecule that reduces expression of a target nucleic acid by an RNA interference (RNAi)-based mechanism. Certain exemplary RNA molecules suitable for RNAi include, but are not limited to, short interfering RNA (siRNAs), short hairpin RNA (shRNAs), microRNA, tiny non-coding RNA (tncRNA), and small modulatory RNA (smRNA) molecules (see, e.g., Novina and Sharp, Nature 430:161-164, 2004).

In certain cases, the inhibitory antisense or double stranded oligonucleotides comprise at least one modified base moiety that is selected from the group including, but not limited to, 5-fluorouracil, 5-chlorouracil, 5-bromouracil, 5-iodouracil, hypoxanthine, xanthine, 5-(carboxyhydroxylmethyl) uracil, dihydrouracil, 5-methoxyuracil, 5-carboxymethylaminomethyl-2-thiouridine, 5-carboxymethylaminomethyluracil, 5-methoxyaminomethyl-2-thiouracil, 5-methyl-2-thiouracil, 5-methyluracil, 2-thiouracil, 4-thiouracil, pseudouracil, uracil-5-oxyacetic acid (v), uracil-5-oxyacetic acid methylester, 3-(3-amino-3-N-2-carboxypropyl) uracil, methylaminomethyluracil, 5′-methoxycarboxymethyluracil, inosine, 1-methylinosine, N6-adenine, N-6-isopentenyladenine, 2-methyladenine, 2-methylthio-N-6-isopentenyladenine, queosine, beta-D-galactosylqueosine, β-D-mannosylqueosine, 1-methylguanine, 2-methylguanine, 7-methylguanine, 2,2-dimethylguanine, 3-methylcytosine, 5-methylcytosine, 4-acetylcytosine, 2-thiocytosine, wybutoxosine, (acp3)w, and 2,6-diaminopurine.

In some cases the antisense oligonucleotides comprise at least one modified sugar moiety selected from the group including, but not limited to, arabinose, 2-fluoroarabinose, xylulose, and hexose. In other cases the antisense oligonucleotides comprise at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof. In yet other embodiments of the present invention, the antisense oligonucleotides are .alpha.-anomeric oligonucleotides. An .alpha.-anomeric oligonucleotide forms specific double-stranded hybrids with complementary RNA in which, contrary to the usual .beta.-units, the strands run parallel to each other (Gautier et al., Nucl. Acids Res. 15:6625-6641, 1987). The oligonucleotide can also be a 2′-0-methylribonucleotide (Inoue et al., Nucl. Acids Res. 15:6131-6148, 1987), or a chimeric RNA-DNA analogue (Inoue et al., FEBS Lett. 215:327-330, 1987). Alternatively, double stranded RNA can be used to disrupt the expression and function of TSHβ.

The activity of an antisense nucleic acid, such as an antisense DNA or siRNA molecule, is often affected by the secondary structure of the target mRNA (see, e.g., Vickers et al., J. Biol. Chem. 278:7108-7118, 2003). Thus, an antisense nucleic acid is selected that is complementary to a region of a target mRNA that is available for base-pairing. A suitable region of a target mRNA can be identified by performing a “gene walk”, e.g., by empirically testing a number of antisense oligonucleotides for their ability to hybridize to various regions along a target mRNA and/or to reduce target mRNA expression (see, e.g., Vickers et al., supra, and Hill et al., Cell Mol. Biol. 21:728-737, 1999). Alternatively, a suitable region of a target mRNA is identified using an mRNA secondary structure prediction program or related algorithm to identify regions of a target mRNA that do not hybridize to any other regions of the target mRNA (see, e.g., Hill et al., supra). A combination of the above methods are used to identify a suitable region of a target mRNA. See, e.g., FIG. 12. Several software systems exist to compute siRNA sequences, these include, but are not limited to, siDirect™, HuSiDa™, siRNAdb™, siSearch™, SpecificityServer™ and miRacle™.

Also included in some embodiments of the present disclosure are: (a) DNA vectors that contain any of the foregoing TSHβ protein coding sequences and/or their complements (i.e., antisense), such as pSilencer™ 4.1-CMV puro expression vector depicted in FIG. 12B for generating an shRNA used for RNAi inhibition of murine TSHβ coding sequences; (b) DNA expression vectors that contain any of the foregoing TSHβ protein coding sequences operatively associated with a regulatory element that directs the expression of the coding sequences (for example, baculovirus as described in U.S. Pat. No. 5,869,336, herein incorporated by reference); (c) genetically engineered host cells that contain any of the foregoing TSHβ coding sequences operatively associated with a regulatory element that directs the expression of the coding sequences in the host cell; and (d) genetically engineered host cells that express an endogenous TSHβ protein sequence under the control of an exogenously introduced regulatory element (i.e., gene activation). As used herein, regulatory elements include, but are not limited to, inducible and non-inducible promoters, enhancers, operators, and other elements known to those skilled in the art that drive and regulate expression. Such regulatory elements include, but are not limited to, the cytomegalovirus (hCMV) immediate early gene, regulatable, viral elements (particularly retroviral LTR promoters), the early or late promoters of SV40 or adenovirus, the lac system, the trp system, the TAC system, the TRC system, the major operator and promoter regions of phage lambda, the control regions of fd coat protein, the promoter for 3-phosphoglycerate kinase (PGK), the promoters of acid phosphatase, and the promoters of the yeast α-mating factors.

Antibodies, Antagonists and Agonists of TSHβ

In some applications, the present disclosure pertains to antibodies and anti-idiotypic antibodies (including Fab fragments), antagonists and agonists of a TSHβ protein, as well as compounds or nucleotide constructs that inhibit expression of a TSHβ protein sequence (transcription factor inhibitors, antisense and ribozyme molecules, or open reading frame sequence or regulatory sequence replacement constructs), or promote the expression of a TSHβ protein (e.g., expression constructs in which TSHβ protein coding sequences are operatively associated with expression control elements such as promoters, promoter/enhancers, etc.).

In some applications variant TSHβ proteins or TSHβ peptides, TSHβ fusion proteins, TSHβ nucleotide sequences, antibodies, antagonists and agonists are useful for the detection of mutant variant TSHβ proteins, or inappropriately expressed variant TSHβ proteins, for the diagnosis of TSHβ-related disorders.

The variant TSHβ proteins or peptides, TSH fusion proteins, TSHβ nucleotide sequences, host cell expression systems, antibodies, antagonists, agonists and genetically engineered cells and animals are used for screening for drugs (or high throughput screening of combinatorial libraries) effective in the treatment of the symptomatic or phenotypic manifestations of perturbing the normal function of a TSHβ protein in the body. The use of engineered host cells and/or animals may offer an advantage in that such systems allow not only for the identification of compounds that bind to the endogenous receptor for a TSH protein, but identify compounds that trigger TSHβ protein-mediated activities or pathways.

In some applications, the TSHβ protein products are used as therapeutics. For example, soluble derivatives such as TSHβ protein peptides/domains corresponding to variant TSHB proteins, TSHβ fusion protein products (especially TSHβ protein-Ig fusion proteins, i.e., fusions of a TSHβ protein, or a domain of a TSHβ protein, to an IgFc), TSHβ protein antibodies and anti-idiotypic antibodies (including Fab fragments), antagonists or agonists (including compounds that modulate or act on downstream targets in a TSHβ protein-mediated pathway) are used to directly treat TSHβ-related disorders. For instance, the administration of an effective amount of a soluble TSHβ protein, a TSHβ protein-IgFc fusion protein, or an anti-idiotypic antibody (or its Fab) that mimics the TSHβ protein, could activate or effectively antagonize an endogenous TSHβ protein activity. Nucleotide constructs encoding such TSHβ protein products are used to genetically engineer host cells to express such products in vivo; these genetically engineered cells function as “bioreactors” in the body delivering a continuous supply of TSHβ protein, TSHβ peptide, or TSHβ fusion protein to the body. Nucleotide constructs encoding functional variant TSHβ proteins, mutant variant TSH/3 proteins, as well as antisense and ribozyme molecules are used in “gene therapy” approaches for the modulation of TSHβ expression. Thus, also included are pharmaceutical formulations and methods for treating TSHβ-related disorders.

Some applications include cells that contain a disrupted TSHβ gene. There are a variety of techniques that can be used to disrupt genes in cells, and especially ES cells. Examples of such methods are described in co-pending U.S. patent application Ser. No. 08/728,963, and U.S. Pat. Nos. 5,789,215, 5,487,992, 5,627,059, 5,631,153, 6,087,555, 6,136,566, 6,139,833, and 6,207,371.

The cDNA sequences (SEQ ID NOS: 3 and 7) and the corresponding deduced amino acid sequences of the described variant TSHβ proteins (SEQ ID NOS: 4 and 8) and the known sequence of mouse and human TSHβ (SEQ ID NOS: 1, 2, 5 and 6 and 7) are presented in the Sequence Listing. SEQ ID NO:1 is the nucleic acid sequence that encodes native mouse TSHβ whose amino acid sequence is shown in SEQ ID NO: 2. SEQ ID NO:3 is the nucleic acid sequence that encodes variant mouse TSHβ whose amino acid sequence is shown in SEQ ID NO: 4. SEQ ID NO:5 is the nucleic acid sequence that encodes native human TSHβ whose amino acid sequence is shown in SEQ ID NO: 6. SEQ ID NO:7 is the nucleic acid sequence that encodes variant human TSHβ whose amino acid sequence is shown in SEQ ID NO: 8.

An additional application of the described novel human polynucleotide sequences is their use in the molecular mutagenesis/evolution of proteins that are at least partially encoded by the described novel sequences using, for example, polynucleotide shuffling or related methodologies. Such approaches are described in U.S. Pat. Nos. 5,830,721 and 5,837,458, which are herein incorporated by reference in their entirety.

Transgenic Animals

In some cases TSHβ protein gene products are expressed in transgenic animals. Animals of any species, except humans, including, but not limited to, worms, mice, rats, rabbits, guinea pigs, pigs, micro-pigs, birds, goats, and non-human primates, e.g., baboons, monkeys, and chimpanzees, may be used to generate TSHβ protein transgenic animals.

Any technique known in the art may be used to introduce a TSHβ protein transgene into animals to produce the founder lines of transgenic animals. Such techniques include, but are not limited to, pronuclear microinjection (Hoppe and Wagner, 1989, U.S. Pat. No. 4,873,191); retrovirus-mediated gene transfer into germ lines (Van der Putten et al., 1985, Proc. Natl. Acad. Sci. USA 82:6148-6152); gene targeting in embryonic stem cells (Thompson et al., 1989, Cell 56:313-321); electroporation of embryos (Lo, 1983, Mol. Cell. Biol. 3:1803-1814); and sperm-mediated gene transfer (Lavitrano et al., 1989, Cell 57:717-723); etc. For a review of such techniques, see Gordon, 1989, Transgenic Animals, Intl. Rev. Cytol. 115:171-229, which is incorporated by reference herein in its entirety.

Also provided are transgenic animals that carry a TSHβ protein transgene in all their cells, as well as animals that carry a transgene in some, but not all of their cells, i.e., mosaic animals or somatic cell transgenic animals. A transgene may be integrated as a single transgene, or in concatamers, e.g., head-to-head tandems or head-to-tail tandems. A transgene may also be selectively introduced into and activated in a particular cell-type by following, for example, the teaching of Lasko et al., 1992, Proc. Natl. Acad. Sci. USA 89:6232-6236. The regulatory sequences required for such a cell-type specific activation will depend upon the particular cell-type of interest, and will be apparent to those of skill in the art.

When it is desired that a TSHβ protein transgene be integrated into the chromosomal site of the endogenous TSHβ protein gene, gene targeting is preferred. Briefly, when such a technique is to be utilized, vectors containing some nucleotide sequences homologous to the endogenous TSHβ protein gene are designed for the purpose of integrating, via homologous recombination with chromosomal sequences, into and disrupting the function of the nucleotide sequence of the endogenous TSHβ protein gene (i.e., “knockout” animals).

The transgene can also be selectively introduced into a particular cell-type, thus inactivating the endogenous TSHβ protein gene in only that cell-type, by following, for example, the teaching of Gu et al., 1994, Science 265:103-106. The regulatory sequences required for such a cell-type specific inactivation will depend upon the particular cell-type of interest, and will be apparent to those of skill in the art.

Once transgenic animals have been generated, the expression of the recombinant TSHβ protein gene may be assayed utilizing standard techniques. Initial screening is accomplished by Southern blot analysis or using PCR techniques to analyze animal tissue to assay whether integration of the transgene has taken place. The level of mRNA expression of the transgene in the tissues of the transgenic animals is assessed using techniques that include, but are not limited to, Northern blot analysis of tissue samples obtained from the animal, in situ hybridization analysis, and RT-PCR. Samples of TSHβ protein gene-expressing tissue are evaluated immunocytochemically using antibodies specific for the TSHβ protein transgene product.

The some applications, “knock-in” animals are used. Knock-in animals are those in which a polynucleotide sequence (i.e., a gene or a cDNA) that the animal does not naturally have in its genome is inserted in such a way that it is expressed. Examples include, but are not limited to, a human gene or cDNA used to replace its murine ortholog in the mouse, a murine cDNA used to replace the murine gene in the mouse, and a human gene or cDNA or murine cDNA that is tagged with a reporter construct used to replace the murine ortholog or gene in the mouse. Such replacements occur at the locus of the murine ortholog or gene, or at another specific site. Such knock-in animals are useful for the in vivo study, testing and validation of, intra alia, human drug targets, as well as for compounds that are directed at the same, and therapeutic proteins.

Variant TSHβ Proteins

Variant TSHβ proteins, TSHβ polypeptides, TSHβ peptide fragments, mutated, truncated, or deleted forms of the variant TSHβ proteins, and/or TSHβ fusion proteins can be prepared for a variety of uses. These uses include, but are not limited to, the generation of antibodies, as reagents in diagnostic assays, for the identification of other cellular gene products related to a TSHβ protein, and as reagents in assays for screening for compounds that can be used as pharmaceutical reagents useful in the therapeutic treatment of TSHβ-related disorder. By way of example, but not limitation, assays utilizing variant TSHβ are used to diagnose and screen for thyroid disorders, to screen newborns for an underactive thyroid, monitor thyroid replacement therapy in people with hypothyroidism, to diagnose and monitor infertility problems, and to treat or diagnose TSHβ-related disorders.

Given the similar information and expression data, the described variant TSHβ proteins can be targeted (by drugs, oligos, antibodies, etc.) in order to treat TSHβ-related disorders, or to therapeutically augment the efficacy of, for example, chemotherapeutic agents used in the treatment of cancer.

The Sequence Listing discloses the amino acid sequences encoded by the described TSHβ polynucleotides. The described TSHβ amino acid sequences include the amino acid sequences presented in the Sequence Listing, as well as analogues and derivatives thereof. Further, corresponding TSHβ protein homologues from other species are encompassed by the invention. In fact, any TSHβ protein encoded by the TSHβ nucleotide sequences described herein are within the scope of the invention, as are any novel polynucleotide sequences encoding all or any novel portion of an amino acid sequence presented in the Sequence Listing. The degenerate nature of the genetic code is well-known, and, accordingly, each amino acid presented in the Sequence Listing is generically representative of the well-known nucleic acid “triplet” codon, or in many cases codons, that can encode the amino acid. As such, as contemplated herein, the amino acid sequences presented in the Sequence Listing, when taken together with the genetic code (see, for example, Table 4-1 at page 109 of “Molecular Cell Biology”, 1986, J. Darnell et al., eds., Scientific American Books, New York, N.Y., herein incorporated by reference), are generically representative of all the various permutations and combinations of nucleic acid sequences that can encode such amino acid sequences.

Also encompassed are proteins that are functionally equivalent to the variant TSHβ proteins encoded by the presently described nucleotide sequences, as judged by any of a number of criteria, including, but not limited to, the ability to form a heterodimer with TSHα, to bind the TSH, the ability to effect an identical or complementary downstream pathway, or a change in cellular metabolism (e.g., proteolytic activity, ion flux, tyrosine phosphorylation, etc.). Such functionally equivalent variant TSHβ proteins include, but are not limited to, additions or substitutions of amino acid residues within the amino acid sequence encoded by the TSHβ nucleotide sequences described herein, but that result in a silent change, thus producing a functionally equivalent expression product. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Expression and Purification of TSHβ

A variety of host-expression vector systems can be used to express the native and variant TSHβ nucleotide sequences of the invention and some are detailed in the Examples below. Such expression systems also encompass engineered host cells that express a TSHβ protein, or functional equivalent, in situ. Purification or enrichment of a TSHβ protein from such expression systems can be accomplished using appropriate detergents and lipid micelles and methods well-known to those skilled in the art. However, such engineered host cells themselves may be used in situations where it is important not only to retain the structural and functional characteristics of a TSHβ protein, but to assess biological activity, e.g., in certain drug screening assays.

The expression systems that may be used for purposes of the invention include, but are not limited to, microorganisms such as bacteria (e.g., E. coli, B. subtilis) transformed with recombinant bacteriophage DNA, plasmid DNA or cosmid DNA expression vectors containing TSHβ nucleotide sequences; yeast (e.g., Saccharomyces, Pichia) transformed with recombinant yeast expression vectors containing TSHβ nucleotide sequences; insect cell systems infected with recombinant virus expression vectors (e.g., baculovirus) containing TSHβ nucleotide sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing TSHβ nucleotide sequences; or mammalian cell systems (e.g., COS, CHO, BHK, 293, 3T3) harboring recombinant expression constructs containing TSHβ nucleotide sequences and promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the TSHβ protein product being expressed. For example, when a large quantity of such a protein is to be produced for the generation of pharmaceutical compositions of, or containing, a TSHβ protein, or for raising antibodies to a TSHβ protein, vectors that direct the expression of high levels of fusion protein products that are readily purified may be desirable. Such vectors include, but are not limited to, the E. coli expression vector pUR278 (Ruther et al., 1983, EMBO J. 2:1791), in which a TSHβ protein coding sequence may be ligated individually into the vector in-frame with the lacZ coding region so that a fusion protein is produced; pIN vectors (Inouye and Inouye, 1985, Nucleic Acids Res. 13:3101-3109; Van Heeke and Schuster, 1989, J. Biol. Chem. 264:5503-5509); and the like. pGEX® vectors (Pharmacia® or American Type Culture Collection®) can also be used to express foreign polypeptides as fusion proteins with glutathione S-transferase (GST). In general, such fusion proteins are soluble and can easily be purified from lysed cells by adsorption to glutathione-agarose beads, followed by elution in the presence of free glutathione. The PGEX® vectors are designed to include thrombin or factor Xa protease cleavage sites so that the cloned target expression product can be released from the GST moiety.

In an exemplary insect system, Autographa californica nuclear polyhedrosis virus (AcNPV) is used as a vector to express foreign polynucleotide sequences. The virus grows in Spodoptera frugiperda cells. A TSHβ protein coding sequence can be cloned individually into a non-essential region (for example the polyhedrin gene) of the virus and placed under control of an AcNPV promoter (for example the polyhedrin promoter). Successful insertion of a TSHβ protein coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted sequence is expressed (e.g., see Smith et al., 1983, J. Virol. 46:584; Smith, U.S. Pat. No. 4,215,051).

In mammalian host cells, a number of viral-based expression systems may be utilized. In cases where an adenovirus is used as an expression vector, the TSHβ nucleotide sequence of interest may be ligated to an adenovirus transcription/translation control complex, e.g., the late promoter and tripartite leader sequence. This chimeric sequence may then be inserted in the adenovirus genome by in vitro or in vivo recombination. Insertion in a non-essential region of the viral genome (e.g., region E1 or E3) will result in a recombinant virus that is viable and capable of expressing a TSHβ protein product in infected hosts (e.g., see Logan and Shenk, 1984, Proc. Natl. Acad. Sci. USA 81:3655-3659). Specific initiation signals may also be required for efficient translation of inserted TSHβ nucleotide sequences. These signals include the ATG initiation codon and adjacent sequences. In cases where an entire TSHβ protein gene or cDNA, including its own initiation codon and adjacent sequences, is inserted into the appropriate expression vector, no additional translational control signals may be needed. However, in cases where only a portion of a TSHβ protein coding sequence is inserted, exogenous translational control signals, including, perhaps, the ATG initiation codon, may be provided. Furthermore, the initiation codon should be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bitter et al., 1987, Methods in Enzymol. 153:516-544).

In addition, a host cell strain may be chosen that modulates the expression of the inserted sequences, or modifies and processes the expression product in the specific fashion desired. Such modifications (e.g., glycosylation) and processing (e.g., cleavage) of protein products may be important for the function of the protein. Different host cells have characteristic and specific mechanisms for the post-translational processing and modification of proteins and expression products. Appropriate cell lines or host systems can be chosen to ensure the desired modification and processing of the foreign protein expressed. To this end, eukaryotic host cells that possess the cellular machinery for the desired processing of the primary transcript, glycosylation, and phosphorylation of the expression product may be used. Such mammalian host cells include, but are not limited to, CHO, VERO, BHK, HeLa, COS, MDCK, 293, 3T3, WI38, and in particular, human cell lines.

For long-term, high-yield production of recombinant proteins, stable expression is preferred. For example, cell lines that stably express the TSHβ protein sequences described herein are engineered. Rather than using expression vectors that contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells may be allowed to grow for 1-2 days in an enriched media, and then switched to a selective media. The selectable marker in the recombinant plasmid confers resistance to the selection and allows cells to stably integrate the plasmid into their chromosomes and grow to form foci, which in turn can be cloned and expanded into cell lines. This method may advantageously be used to engineer cell lines that express a TSHβ protein product. Such engineered cell lines may be particularly useful in screening and evaluation of compounds that affect the endogenous activity of a TSHβ protein product.

A number of selection systems may be used, including, but not limited to, the Herpes simplex virus thymidine kinase (Wigler et al., 1977, Cell 11:223), hypoxanthine-guanine phosphoribosyltransferase (Szybalska and Szybalski, 1962, Proc. Natl. Acad. Sci. USA 48:2026), and adenine phosphoribosyltransferase (Lowy et al., 1980, Cell 22:817) genes, which can be employed in tk⁻, hgprt⁻ or aprt⁻ cells, respectively. Also, antimetabolite resistance can be used as the basis of selection for the following genes: dhfr, which confers resistance to methotrexate (Wigler et al., 1980, Proc. Natl. Acad. Sci. USA 77:3567; O'Hare et al., 1981, Proc. Natl. Acad. Sci. USA 78:1527); gpt, which confers resistance to mycophenolic acid (Mulligan and Berg, 1981, Proc. Natl. Acad. Sci. USA 78:2072); neo, which confers resistance to the aminoglycoside G-418 (Colberre-Garapin et al., 1981, J. Mol. Biol. 150:1); and hygro, which confers resistance to hygromycin (Santerre et al., 1984, Gene 30:147).

Alternatively, any fusion protein can be readily purified by utilizing an antibody specific for the fusion protein being expressed. Another exemplary system allows for the ready purification of non-denatured fusion proteins expressed in human cell lines (Janknecht et al., 1991, Proc. Natl. Acad. Sci. USA 88:8972-8976). In this system, the sequence of interest is subcloned into a vaccinia recombination plasmid such that the sequence's open reading frame is translationally fused to an amino-terminal tag consisting of six histidine residues. Extracts from cells infected with recombinant vaccinia virus are loaded onto Ni²⁺ nitriloacetic acid-agarose columns, and histidine-tagged proteins are selectively eluted with imidazole-containing buffers.

Also encompassed are fusion proteins that direct a TSHβ protein to a target organ and/or facilitate transport across the membrane into the cytosol. Conjugation of variant TSHβ proteins to antibody molecules or their Fab fragments could be used to target cells bearing a particular epitope. Attaching an appropriate signal sequence to a TSHβ protein would also transport a TSHβ protein to a desired location within the cell. Alternatively, targeting of a TSHβ protein or its nucleic acid sequence might be achieved using liposome or lipid complex based delivery systems. Such technologies are described in “Liposomes: A Practical Approach”, New, R. R. C., ed., Oxford University Press, N.Y., and in U.S. Pat. Nos. 4,594,595, 5,459,127, 5,948,767 and 6,110,490 and their respective disclosures, which are herein incorporated by reference in their entirety. Additionally embodied are novel protein constructs engineered in such a way that they facilitate transport of variant TSHβ proteins to a target site or desired organ, where they cross the cell membrane and/or the nucleus where the variant TSHβ proteins can exert their functional activity. This goal may be achieved by coupling of a TSHβ protein to a cytokine or other ligand that provides targeting specificity, and/or to a protein transducing domain (see generally U.S. Provisional Patent Application Ser. Nos. 60/111,701 and 60/056,713, both of which are herein incorporated by reference, for examples of such transducing sequences), to facilitate passage across cellular membranes, and can optionally be engineered to include nuclear localization signals.

Additionally contemplated are TSHβ oligopeptides that are modeled on an amino acid sequence first described in the Sequence Listing. Such protein oligopeptides are generally between about 10 to about 100 amino acids long, or between about 16 to about 80 amino acids long, or between about 20 to about 35 amino acids long, or any variation or combination of sizes represented therein that incorporate a contiguous region of sequence first disclosed in the Sequence Listing. Such TSHβ protein oligopeptides can be of any length disclosed within the above ranges, and can initiate at any amino acid position represented in the Sequence Listing.

Also contemplated are “substantially isolated” or “substantially pure” proteins or polypeptides. The phrase “substantially isolated” or “substantially pure” protein or polypeptide is meant to describe a protein or polypeptide that has been separated from at least some of those components that naturally accompany it. Typically, the protein or polypeptide is substantially isolated or pure when it is at least 60%, by weight, free from the proteins and other naturally-occurring organic molecules with which it is naturally associated in vivo. Preferably, the purity of the preparation is at least 75%, more preferably at least 90%, and most preferably at least 99%, by weight. A substantially isolated or pure protein or polypeptide may be obtained, for example, by extraction from a natural source, by expression of a recombinant nucleic acid encoding the protein or polypeptide, or by chemically synthesizing the protein or polypeptide.

Purity can be measured by any appropriate method, e.g., column chromatography such as immunoaffinity chromatography using an antibody specific for the protein or polypeptide, polyacrylamide gel electrophoresis, or HPLC analysis. A protein or polypeptide is substantially free of naturally associated components when it is separated from at least some of those contaminants that accompany it in its natural state. Thus, a polypeptide that is chemically synthesized or produced in a cellular system different from the cell from which it naturally originates will be, by definition, substantially free from its naturally associated components. Accordingly, substantially isolated or pure proteins or polypeptides include eukaryotic proteins synthesized in E. coli, other prokaryotes, or any other organism in which they do not naturally occur.

TSHβ Epitopes and Immunogens

The term “epitope” refers to any polypeptide determinant capable of selectively binding to an immunoglobulin or a T-cell receptor. In general, an epitope is a region of an antigen that is selectively bound by an antibody. In certain cases, an epitope may include chemically active surface groupings of molecules such as amino acids, sugar side chains, phosphoryl, and/or sulfonyl groups. Additionally, an epitope may have specific three dimensional structural characteristics (e.g., a “conformational” epitope) and/or specific charge characteristics

An epitope is defined as “the same” as another epitope if a particular antibody selectively binds to both epitopes. In certain cases, polypeptides having different primary amino acid sequences may comprise epitopes that are the same, and epitopes that are the same may have different primary amino acid sequences. Different antibodies are said to bind to the same epitope if they compete for selective binding to that epitope.

One may identify epitopes from primary amino acid sequences on the basis of hydrophilicity. These regions are also referred to as “epitopic core regions.” In general, native or variant TSHβ peptides selected for immunizing an animal comprise one or more epitopes, as such peptides are likely to be immunogenic. In general, peptide immunogens and epitopes are those that are predicted to be hydrophilic and/or likely to be exposed on the surface of native or variant TSHβ in its folded state. In certain embodiments, peptide segments that are predicted to form β-turns, and are therefore likely to be exposed on the surface of a protein, may be selected as immunogens. Alternatively, it is not necessary that the epitope be expressed on the surface of the protein. Many immunological techniques utilize the addition of reagents to facilitate protein unfolding, thereby unmasking epitopes that were unavailable prior to the manipulation. Guidance for selecting suitable immunogenic peptides and related techniques are provided, for example, in “Current Protocols in Molecular Biology”, Vol. 1 and 2 (Ausubel et al., eds., Green Publishing Associates, Incorporated, and John Wiley & Sons, Incorporated, New York, N.Y., 1989) Ch. 11.14, and “Antibodies: A Laboratory Manual” (Harlow and Lane, eds., Cold Spring Harbor Laboratory Press, Cold Spring Harbor, N.Y., 1988) Ch. 5.

Certain algorithms are known to those skilled in the art for predicting whether a peptide segment of a protein is hydrophilic, and therefore likely to be exposed on the surface of the protein. These algorithms use the primary sequence information of a protein to make such predictions, and are based on the method of, for example, Hopp and Woods, Proc. Natl. Acad. Sci. USA 78:3824-3828, 1981, or Kyte and Doolittle, supra. Certain exemplary algorithms are known to those skilled in the art for predicting the secondary structure of a protein based on the primary amino acid sequence of the protein (see, e.g., Corrigan and Huang, Comput. Programs Biomed. 15:163-168, 1982, Chou and Fasman, Ann. Rev. Biochem. 47:251-276, 1978, Moult, Curr. Opin. Biotechnol. 7:422-427, 1996, Chou and Fasman, Biochemistry 13: 222-245, 1974, Chou and Fasman, Biochemistry 13:211-222, 1974, Chou and Fasman, Adv. Enzymol. Relat. Areas Mol. Biol. 47: 45-148, 1978, and Chou and Fasman, Biophys. J. 26: 367-383, 1979).

Moreover, computer programs are currently available to assist with predicting secondary structure. One method of predicting secondary structure is based upon homology modeling. For example, two polypeptides or proteins that have a sequence identity of greater than 30%, or similarity greater than 40%, often have similar structural topologies. The growth of the Protein Structural Database (PSDB); Berman et al., Nucleic Acids Res. 28: 235-242, 2000) and the Protein Data Bank (PDB) has provided enhanced predictability of secondary structure, including the potential number of folds within the structure of a polypeptide (see, e.g., Holm and Sander, Nucleic Acids Res. 27: 244-247, 1999). It has been suggested there are a limited number of folds in a given polypeptide or protein, and once a critical number of structures have been resolved, structural prediction will become much more accurate (Brenner et al., Curr. Opin. Struct. Biol. 7: 369-376, 1997). Additional methods of predicting secondary structure include “threading” (see, e.g., Jones, Curr. Opin. Struct. Biol. 7:377-387, 1997, and Sippl and Flockner, Structure 4:15-19, 1996), “profile analysis” (see, e.g., Bowie et al., Science 253:164-170, 1991, Gribskov et al., Meth. Enzymol. 183:146-159, 1990, and Gribskov et al., Proc. Natl. Acad. Sci. USA 84:4355-4358, 1987), and “evolutionary linkage” (see, e.g., Holm and Sander, 1999, supra, and Brenner et al., 1997, supra).

The use of antibodies that selectively bind to one or more epitopes of TSHβ or epitopes of conserved variants of TSHβ, or to splice variant isoforms of TSHβ and their fragments are also contemplated, particularly for use in the immunoassays described herein. Antibodies for use in these immunoassays include those available commercially. Such antibodies include, but are not limited to, polyclonal antibodies, monoclonal antibodies (mAbs), humanized antibodies, human-engineered antibodies, fully human antibodies, chimeric antibodies, single chain antibodies, Fab fragments, F(ab′)2 fragments, fragments produced by a Fab expression library, anti-idiotypic (anti-Id) antibodies, catalytic antibodies, and epitope-binding fragments of any of the above. In some applications, the antibodies, or fragments thereof, will preferentially bind to native or variant TSHβ, as opposed to other proteins. In such cases, the antibodies, or fragments thereof, selectively bind to native or variant TSHβ with a higher affinity or avidity than they bind to other proteins.

An antibody “selectively binds” an antigen when it preferentially recognizes the antigen in a complex mixture of proteins and/or other macromolecules. The antibodies employed in some of the methods disclosed herein comprise an antigen-binding site that selectively binds to a particular epitope. Such antibodies can be capable of binding to different antigens, so long as the different antigens comprise that particular epitope. In some applications, homologous proteins from different species comprise the same epitope. In various applications, an antibody selectively binds an antigen when the dissociation constant (K_{D}) is 1 uM, or when the dissociation constant is 100 nM, or when the dissociation constant is 10 nM, for example.

Antibodies that selectively bind to native or variant TSHβ may be used, for example, in the detection, enrichment, purification or isolation of cells bearing these cell surface markers.

A native antibody typically has a tetrameric structure comprising two identical pairs of polypeptide chains, each pair having one light chain (typically about 25 kDa) and one heavy chain (typically about 50-70 kDa). In a native antibody, a heavy chain comprises a variable region, V_{H}, and three constant regions, C_{H}1, C_{H}2, and C_{H}3. The V_{H} domain is at the amino-terminus of the heavy chain, and the C_{H}3 domain is at the carboxy-terminus. In a native antibody, a light chain comprises a variable region, V_{L}, and a constant region, C_{L}. The variable region of the light chain is at the amino-terminus of the light chain. In a native antibody, the variable regions of each light/heavy chain pair typically form the antigen binding site. The constant regions are typically responsible for effector function.

In humans, for example, native human light chains are typically classified as kappa and lambda light chains. Native human heavy chains are typically classified as mu, delta, gamma, alpha, or epsilon, and define the isotype of the antibody as IgM, IgD, IgG, IgA, and IgE, respectively. IgG has subclasses, including, but not limited to, IgG1, IgG2, IgG3, and IgG4. IgM has subclasses including, but not limited to, IgM1 and IgM2. IgA has subclasses including, but not limited to, IgA1 and IgA2. Within native human light and heavy chains, the variable and constant regions are typically joined by a “J” region of about 12 or more amino acids, with the heavy chain also including a “D” region of about 10 more amino acids (“Fundamental Immunology”, 2^{nd} Ed., Ch. 7 (Paul, ed., Raven Press, New York, N.Y., 1989)). In various applications, the antibodies used in an immunoassay are of any of the isotypes or isotype subclasses set forth above.

In a native antibody, the variable regions typically exhibit the same general structure in which relatively conserved framework regions (FRs) are joined by three hypervariable regions, also called complementarity determining regions (CDRs). The CDRs from the two chains of each pair typically are aligned by the framework regions, which may enable binding to a specific epitope. From N-terminus to C-terminus, both light and heavy chain variable regions typically comprise the domains FR1, CDR1, FR2, CDR2, FR3, CDR3 and FR4. The CDRs on the heavy chain are referred to as H1, H2, and H3, while the CDRs on the light chain are referred to as L1, L2, and L3. Typically, CDR3 is the greatest source of molecular diversity within the antigen binding site. For example, H3, in certain instances, can be as short as two amino acid residues or greater than 26. The assignment of amino acids to each domain is typically in accordance with the definitions in “Sequences of Proteins of Immunological Interest” (Kabat et al., eds., National Institutes of Health, Publication No. 91-3242, 5^{th} Ed., United States Department of Health and Human Services, Bethesda, Md., 1991), Chothia and Lesk, J. Mol. Biol. 196:901-917, 1987, or Chothia et al., Nature 342:878-883, 1989. In the present application, the term “CDR” refers to a CDR from either the light or heavy chain, unless otherwise specified.

In addition to TSH/3 antibodies and TSHβ kits, as are known to those of skill in the art and may be commercially available, antibodies for use in the TSHβ immunoassays disclosed herein include those that are generated de novo.

For the production of antibodies, various host animals, such as but not limited to chickens, hamsters, guinea pigs, rabbits, sheep, goats, horses, may be immunized by injection with a native or variant TSHβ protein, polypeptide, or peptide, a truncated TSHβ polypeptide, a functional equivalent of TSHβ, a mutant of TSHβ, an antigenic fragment thereof, or combinations thereof. Such host animals may include, but are not limited to, rabbits, mice, and rats, and TSHβ “knock-out” variants of the same. In addition, antibodies can be produced by immunizing female birds (chickens, for example) and harvesting the IgY antibodies present in their eggs. Various adjuvants may be used to increase the immunological response, depending on the host species, including, but not limited to, Freund's adjuvant (complete and incomplete), mineral salts such as aluminum hydroxide or aluminum phosphate, surface active substances, chitosan, lysolecithin, pluronic polyols, polyanions, peptides, oil emulsions, and potentially useful human adjuvants such as BCG (Bacille Calmette-Guerin) and Corynebacterium parvum. Alternatively, the immune response could be enhanced by combination and/or coupling with molecules such as keyhole limpet hemocyanin (KLH), tetanus toxoid, diphtheria toxoid, ovalbumin, cholera toxin, or fragments thereof. Alternatively expression as a fusion protein, such as GST, HIS6, or another suitable fusion protein may be used.

Polyclonal antibodies are heterogeneous populations of antibody molecules, such as those derived from the sera of the immunized animals or by mixing B-cells or monoclonal antibodies. Monoclonal antibodies, which are homogeneous populations of antibodies that arise from a single B-cell or its which selectively bind to a particular antigen or epitope, may be obtained by any technique that provides for the production of antibody molecules by continuous cell lines in culture. These include, but are not limited to, the hybridoma technique (Kohler and Milstein, Nature 256:495-497, 1975, U.S. Pat. No. 4,376,110, and “Antibodies: A Laboratory Manual”, supra, Ch. 6), the human B-cell hybridoma technique (Kozbor and Roder, Immunol. Today 4:72-79, 1983, and Cote et al., Proc. Natl. Acad. Sci. USA 80:2026-2030, 1983), and the EBV-hybridoma technique (Cole et al., Mol. Cell. Biochem. 62:109-120, 1984, and Cole et al., Cancer Res. 44:2750-2753, 1984). A suitable animal, such as a mouse, rat, hamster, monkey, or other mammal, or an avian species, is immunized with an immunogen to produce antibody-secreting cells, including, but not limited to, B-cells, such as lymphocytes or splenocytes. In certain embodiments, lymphocytes (e.g., human lymphocytes) are immunized in vitro to generate antibody-secreting cells (Borrebaeck et al., Proc. Natl. Acad. Sci. USA 85:3995-3999, 1988). The hybridomas producing the monoclonal antibodies that are used in certain embodiments may be cultivated in vitro or in vivo. In some instances, the production of high titer monoclonal antibodies in vivo is the preferred method of producing antibodies for use in a testing method described herein.

For some applications, antibody-secreting cells are fused with an “immortalized” cell line, such as a myeloid-type cell line, to produce hybridoma cells. Hybridoma cells that produce the desired antibodies can be identified, for example, by ELISA, and can then be subcloned and cultured using standard methods, or grown in vivo as ascites tumors in a suitable animal host. For some applications, monoclonal antibodies are isolated from hybridoma culture medium, serum, or ascites fluid using standard separation procedures, such as affinity chromatography (see, e.g., “Antibodies: A Laboratory Manual”, supra, Ch. 8).

In some cases high affinity antibodies are generated using animals that have been genetically engineered to be deficient in native or variant TSHβ production and activity. An example of such knock-out animals (mice) are produced using established gene trapping methods, and viable animals that are genetically homozygous for the genetically engineered native or variant TSHβ mutation are generated and characterized. Given the relatedness of mammalian native or variant TSHβ amino acid sequences, the presently described homozygous knock-out mice (having never seen, and thus never been tolerized to, native or variant TSHβ can be advantageously applied to the generation of antibodies against mammalian TSHβ sequences (i.e., native or variant) will be immunogenic in native or variant TSHβ homozygous knock-out animals). High affinity anti-native or variant TSHβ antibodies generated from such animals can be formulated into immunoassays that are used, as described herein, to identify and treat patients at risk for TSHβ-related disorders.

For example, human monoclonal antibodies are raised in transgenic animals (e.g., mice) that are capable of producing human antibodies (see, e.g., U.S. Pat. Nos. 6,075,181 and 6,114,598, and PCT Patent Application Publication No. WO 98/24893). Human immunoglobulin genes can be introduced (e.g., using yeast artificial chromosomes, human chromosome fragments, or germline integration) into mice in which the endogenous Ig genes have been inactivated (see, e.g., Jakobovits et al., Nature 362:255-258, 1993, Tomizuka et al., Proc. Natl. Acad. Sci. USA 97:722-727, 2000, and Mendez et al., Nat. Genet. 15:146-156, 1997, describing the XenoMouse II® line of transgenic mice), for instance. Additional exemplary methods and transgenic mice suitable for the production of human monoclonal antibodies are described, e.g., in Jakobovits, Curr. Opin. Biotechnol. 6:561-566, 1995, Lonberg and Huszar, Int. Rev. Immunol. 13:65-93, 1995, Fishwild et al., Nat. Biotechnol. 14:845-851, 1996, Green, J. Immunol. Methods 231:11-23, 1999, and Little et al., Immunol. Today 21:364-370, 2000.

In addition, techniques developed for the production of “chimeric antibodies” (Morrison et al., Proc. Natl. Acad. Sci. USA 81:6851-6855, 1984, Neuberger et al., Nature 312:604-608, 1984, and Takeda et al., Nature 314:452-454, 1985), for example by splicing the genes from a mouse antibody molecule of appropriate antigen selectivity together with genes from a human antibody molecule of appropriate biological activity, can be used. A chimeric antibody is a molecule in which different portions are derived from different animal species, such as those having a variable region derived from a murine monoclonal antibody and a human immunoglobulin constant region. Such technologies are described in U.S. Pat. Nos. 6,075,181 and 5,877,397, for example.

Monoclonal antibodies that are employed in some applications for identifying native or variant TSHβ can also be produced by recombinant techniques (see, e.g., U.S. Pat. No. 4,816,567). In such embodiments, nucleic acids encoding monoclonal antibody chains are cloned and expressed in a suitable host cell. For example, RNA can be prepared from cells expressing the desired antibody, such as mature B-cells or hybridoma cells, which can then be used to make cDNA, using standard methods. The cDNA encoding a heavy or light chain polypeptide can be amplified, for example, by PCR, using specific oligonucleotide primers. The cDNA can then be cloned into a suitable expression vector, which is then transformed or transfected into a suitable host cell, such as a host cell that does not endogenously produce antibody.

Transformation or transfection can be accomplished by any known method suitable for introducing polynucleotides into a host cell. Certain exemplary methods include, but are not limited to, packaging the polynucleotide in a virus (or into a viral vector) and transducing a host cell with the virus (or vector) and using certain transfection procedures known in the art, as exemplified by U.S. Pat. Nos. 4,399,216, 4,912,040, 4,740,461, and 4,959,455. In certain embodiments, the transformation procedure used may depend upon the host to be transformed. Various methods for introduction of heterologous polynucleotides into mammalian cells are known in the art and include, but are not limited to, dextran-mediated transfection, calcium phosphate precipitation, polybrene-mediated transfection, protoplast fusion, electroporation, encapsulation of the polynucleotide(s) in liposomes, and direct microinjection of the DNA into nuclei. In embodiments where heavy and light chains are co-expressed in the same host, reconstituted antibody may be isolated.

Alternatively, techniques described for the production of single chain antibodies (Bird et al., Science 242:423-426, 1988, Huston et al., Proc. Natl. Acad. Sci. USA 85:5879-5883, 1988, Ward et al., Nature 341:544-546, 1989, PCT Patent Application Publication No. WO 88/01649, and U.S. Pat. Nos. 4,946,778 and 5,260,203) can be adapted to produce single chain antibodies against TSHβ gene products or epitopes. Single chain antibodies are formed by linking the heavy and light chain fragments of the Fv region via an amino acid bridge, resulting in a single chain polypeptide with an antigen binding region.

In some applications, a method or test kit disclosed herein for identifying and enriching or purifying native or variant TSHβ employs antibody fragments, including, but not limited to, Fab, Fab′, F(ab′)₂, Fv, scFv, Fd, diabodies, and other antibody fragments that retain at least a portion of the variable region of an intact antibody (see, e.g., Hudson and Souriau, Nature Med. 9:129-134, 2003). A Fab fragment comprises one light chain and the C_{H}1 and variable region of one heavy chain. The heavy chain of a Fab molecule cannot form a disulfide bond with another heavy chain molecule. A Fab′ fragment comprises one light chain and one heavy chain that comprises additional constant region, extending between the C_{H}1 and C_{H}2 domains, and can be generated by reducing the disulfide bridges of F(ab′)₂ fragments. An interchain disulfide bond can be formed between two heavy chains of a Fab′ fragment to form a F(ab′)₂ molecule, which can be produced by pepsin digestion of an antibody molecule. A Fv fragment comprises the variable regions from both the heavy and light chains, but lacks the constant regions. In certain instances, a single variable region (one-half of a Fv) may have the ability to recognize and bind antigen, albeit with lower affinity than the Fv. A Fab expression library may also be constructed (Huse et al., Science 246:1275-1281, 1989) to allow rapid and easy identification of monoclonal Fab fragments with the desired selectivity.

Monoclonal antibodies employed in certain embodiments may also be produced using a display-based method. For example, monoclonal antibodies can be produced using phage display techniques (see, e.g., Hoogenboom, Methods Mol. Biol. 178:1-37, 2002, Clackson et al., Nature 352:624-628, 1991, and Marks et al., J. Mol. Biol. 222:581-597, 1991). For example, a library of antibodies can be displayed on the surface of a filamentous phage, such as the nonlytic filamentous phage fd or M13. The antibodies can be antibody fragments, such as scFvs, Fabs, Fvs with an engineered intermolecular disulfide bond to stabilize the V_{H}-V_{L} pair, and diabodies. Using these techniques, antibodies with the desired binding selectivity can then be selected.

For example, in some instances, variable gene repertoires are prepared by PCR amplification of genomic DNA or cDNA derived from the mRNA of antibody-secreting cells, such as B-cells. For example, cDNA encoding the variable regions of heavy and light chains can be amplified by PCR, and the heavy chain cDNA and light chain cDNA cloned into a suitable vector. The heavy chain cDNA and light chain cDNA can be randomly combined during the cloning process, thereby resulting in the assembly of a cDNA library encoding diverse scFvs or Fabs. Alternatively, the heavy chain cDNA and light chain cDNA can be ligated, for example by stepwise cloning, before being cloned into a suitable vector.

Suitable vectors include, but are not limited to, phage display vectors, such as a phagemid vectors. Certain exemplary phagemid vectors, such as pCES1, are known to those skilled in the art. In certain embodiments, cDNA encoding both heavy and light chains is present on the same vector. For example, cDNA encoding scFvs can be cloned in-frame with all or a portion of gene III, which encodes the minor phage coat protein pIII. The phagemid then directs the expression of the scFv-pIII fusion on the phage surface. Alternatively, cDNA encoding heavy chain (or light chain) can be cloned in-frame with all or a portion of gene III, and cDNA encoding light chain (or heavy chain) can be cloned downstream of a signal sequence in the same vector. The signal sequence directs expression of the light chain (or heavy chain) into the periplasm of the host cell, where the heavy and light chains assemble into Fab fragments. In other methods, cDNA encoding heavy chain and cDNA encoding light chain can be present on separate vectors. In these methods, heavy chain and light chain cDNA are cloned separately, one into a phagemid and the other into a phage vector, which both contain signals for in vivo recombination in the host cell. The recombinant phagemid and/or phage vectors are introduced into a suitable bacterial host, such as E. coli. When using certain phagemids, the host can be infected with helper phage to supply phage structural proteins, thereby allowing expression of phage particles carrying the antibody-pIII fusion protein on the phage surface.

“Synthetic” antibody libraries can be constructed using repertoires of variable genes that are rearranged in vitro. For example, individual gene segments encoding heavy or light chains (V-D-J or V-J, respectively) are randomly combined using PCR. Additional sequence diversity can be introduced into the CDRs, such as CDR3 (H3 of the heavy chain), and possibly FRs, by error prone PCR.

“Naïve” or “universal” phage display libraries can be constructed, as described above, using nucleic acids from a naïve (unimmunized) animal, while “immunized” phage display libraries can be constructed, as described above, using nucleic acids from an immunized animal. Exemplary universal human antibody phage display libraries are available from commercial sources, and include, but are not limited to, the HuCAL® series of libraries from MorphoSys AG (Martinstried/Planegg, Germany), libraries from Crucell (Leiden, the Netherlands) using MAbstract® technology, the n-CoDeR™ Fab library from BioInvent International AB (Lund, Sweden), and libraries available from Cambridge Antibody Technology (Cambridge, United Kingdom).

Selection of antibodies having the desired binding selectivity from a phage display library can be achieved by successive panning steps. In panning, library phage preparations are exposed to one or more antigen(s), such as one or more native or variant TSHβ antigen(s). The phage-antigen complexes are then washed, and unbound phage are discarded. The bound phage are recovered, and subsequently amplified by infecting E. coli. Monoclonal antibody-producing phage can be cloned by picking single plaques. In some instances, the above process is repeated one or more times.

The antigen is immobilized on a solid support to allow purification of antigen-binding phage by affinity chromatography. Alternatively, the antigen is biotinylated, thereby allowing the separation of bound phage from unbound phage using streptavidin-coated magnetic beads. In some instances, the antigen is immobilized on cells (for direct panning), in tissue cryosections, or on membranes (e.g., nylon or nitrocellulose membranes). Other variations of these panning procedures may be routinely determined by one skilled in the art. Yeast display systems may also be used to produce monoclonal antibodies. In these systems, an antibody is expressed as a fusion protein with all or a portion of a yeast protein, for example the yeast AGA2 protein, which becomes displayed on the surface of the yeast cell wall. Yeast cells expressing antibodies with the desired binding selectivity can then be identified by exposing the cells to fluorescently labeled antigen, and isolated by flow cytometry (see, e.g., Boder and Wittrup, Nat. Biotechnol. 15:553-557, 1997).

Antibodies that bind native or variant TSHβ may include antibodies that are modified to alter one or more of the properties of the antibody. For some applications, a modified antibody may possess certain advantages over an unmodified antibody, such as increased affinity, for example. An antibody can be modified by linking it to a nonproteinaceous moiety, or by altering the glycosylation state of the antibody, e.g., by altering the number, type, linkage, and/or position of carbohydrate chains on the antibody, or altered so that it is not glycosylated.

In some other modification techniques, one or more chemical moieties may be linked to the amino acid backbone and/or carbohydrate residues of the antibody. Certain exemplary methods for linking a chemical moiety to an antibody include, but are not limited to, acylation reactions or alkylation reactions (see, e.g., Malik et al., Exp. Hematol. 20:1028-1035, 1992, Francis, in “Focus on Growth Factors”, Vol. 3, No. 2, pp. 4-10 (Mediscript, Ltd., London, United Kingdom, 1992), European Patent Application Publication Nos. EP 0 401 384 and EP 0 154 316, and PCT Patent Application Publication Nos. WO 92/16221, WO 95/34326, WO 95/13312, WO 96/11953, and WO 96/19459). These reactions may be used to generate an antibody that is chemically modified at its amino-terminus for use in certain embodiments. An antibody may also be modified by linkage to a detectable label, such as an enzymatic, fluorescent, isotopic or affinity label. Such a detectable label may allow for the detection or isolation of the antibody, and/or the detection of an antigen bound by the antibody in various immunoassays. Depending on the nature of the label, qualitative and/or quantitative measurement of native or variant TSHβ can be made using a colorimeter, a spectrophotometer, an ELISA reader, a fluorometer, or a gamma or scintillation (alpha or beta) counter that detects radioactive decay in assays utilizing isotope labels.

Higher affinity TSHβ antibodies are employed to provide significant advantages in the native or variant TSHβ, as described herein. Potential advantages include, but are not limited to, greater assay sensitivity, increased linearity, and decreased cost of goods. Antibody affinity may, in some cases, determine the formats that are available. The affinity of an antibody for a particular antigen may be increased by subjecting the antibody to affinity maturation (or “directed evolution”) in vitro. In vivo, native antibodies undergo affinity maturation through somatic hypermutation followed by selection. Certain in vitro methods mimic that in vivo process, thereby allowing the production of antibodies having affinities that equal or surpass that of native antibodies.

In certain types of affinity maturation, mutations are introduced into a nucleic acid sequence encoding the variable region of an antibody having the desired binding selectivity (see, e.g., Hudson and Souriau, supra, and Brekke and Sandlie, Nat. Rev. Drug Discov. 2:52-62, 2002). Such mutations can be introduced into the variable region of the heavy chain, light chain, or both, into one or more CDRs, into H3, L3, or both, and/or into one or more FRs. A library of mutations can be created, for example, in a phage, ribosome, or yeast display library, so antibodies with increased affinity may be identified by standard screening methods (see, e.g., Boder et al., Proc. Natl. Acad. Sci. USA 97:10701-10705, 2000, Foote and Eisen, Proc. Natl. Acad. Sci. USA 97:10679-10681, 2000, Hoogenboom, supra, and Hanes et al., Proc. Natl. Acad. Sci. USA 95:14130-14135, 1998).

Mutations can be introduced by site-specific mutagenesis, based on information on the structure of the antibody, e.g., the antigen binding site, or using combinatorial mutagenesis of CDRs. Alternatively, all or a portion of the variable region coding sequence may be randomly mutagenized, e.g., using E. coli mutator cells, homologous gene rearrangement, or error prone PCR. Mutations may also be introduced using “DNA shuffling” (see, e.g., Crameri et al., Nature Med. 2:100-102, 1996, and Fermer et al., Tumour Biol. 25:7-13, 2004).

In addition, “chain shuffling” may be used to generate antibodies with increased affinity. In chain shuffling, one of the chains, e.g., the light chain, is replaced with a repertoire of light chains, while the other chain, e.g., the heavy chain, is unchanged, thus providing selectivity. A library of chain shuffled antibodies can be created, wherein the unchanged heavy chain is expressed in combination with each light chain from the repertoire of light chains. Such libraries may then be screened for antibodies with increased affinity. In particular applications, both the heavy and light chains are sequentially replaced, only the variable regions of the heavy and/or light chains are replaced, or only a portion of the variable regions, e.g., CDRs, of the heavy and/or light chains are replaced (see, e.g., Hudson and Souriau, supra, Brekke and Sandlie, supra, Kang et al., Proc. Natl. Acad. Sci. USA 88:11120-11123, 1991, and Marks et al., Biotechnology (NY) 10:779-783, 1992).

Mouse monoclonal antibodies that selectively bind native or variant human TSHβ or TSHβ from other mammals are subject to sequential chain shuffling. Such monoclonal antibodies include but not limited to, mouse monoclonal antibodies raised against native or variant mouse TSHβ but selectively bind to (i.e., cross-react with) native or variant human TSHβ. For example, the heavy chain of a given mouse monoclonal antibody may be combined with a new repertoire of human light chains, and antibodies with the desired affinity may be selected. The light chains of the selected antibodies may then be combined with a new repertoire of human heavy chains, and antibodies with the desired affinity may be selected. In this manner, human antibodies having the desired antigen binding selectivity and affinity are obtained.

Alternatively, the heavy chain of a given mouse monoclonal antibody may be combined with a new repertoire of human light chains, and antibodies with the desired affinity selected from this first round of shuffling. In addition, the light chain of the original mouse monoclonal antibody is combined with a new repertoire of human heavy chains, and antibodies with the desired affinity selected from this second round of shuffling. Then, human light chains from the antibodies selected in the first round of shuffling are combined with human heavy chains from the antibodies selected in the second round of shuffling. Thus, human antibodies having the desired antigen binding selectivity and affinity may be selected.

Alternatively, a “ribosome display” method may be used that alternates antibody selection with affinity maturation. In the ribosome display method, antibody-encoding nucleic acid is amplified by RT-PCR between the selection steps. Thus, error prone polymerases may be used to introduce mutations into the nucleic acid (see, e.g., Hanes et al., supra).

Antibodies that bind native or variant TSHβ, as disclosed herein, may be screened for binding to native or variant TSHβ (for example, human, mouse, dog, cat, horse) using certain routine methods that detect binding of an antibody to an antigen. In some embodiments, similar methods and assay formats are used to detect variant TSHβ on cells obtained from patients with TSHβ-related disorders. For example, the ability of a monoclonal antibody to bind TSHβ may be assayed by standard immunoblotting methods, such as electrophoresis and Western blotting (see, e.g., Ch. 10.8 in “Current Protocols in Molecular Biology”, Ch. 11.14, Vol. 1 and 2 (Ausubel et al., eds., Green Publishing Associates, Incorporated, and John Wiley & Sons, Incorporated, New York, N.Y., 1989 and “Antibodies: A Laboratory Manual” (Harlow and Lane, eds., Cold Spring Harbor Laboratory Press, Cold Spring Harbor, N.Y., 1988). Alternatively, the ability of a monoclonal antibody to bind TSHβ may be assayed using a competitive binding assay, which evaluates the ability of a candidate antibody to compete with a known anti-TSHβ antibody for binding to native or variant TSHβ, respectively. Competitive binding assays may be performed in various formats including but not limited to ELISA (see, e.g., “Antibodies: A Laboratory Manual”, supra, Ch. 14) the results of which are determined using a colorimeter with one or more fixed wavelengths, or a variable wavelength spectrophotometer, or an ELISA reader, or a fluorometer. In some embodiments, such assays are used to determine the presence of variant TSHβ in patents with TSHβ-related disorders.

A binding assay may be used to quantify the binding kinetics (e.g., rate constant) or the binding affinity (e.g., association or dissociation constant) of an antibody against native or variant TSHβ. The binding kinetics or binding affinity can be determined in the “solid-phase” by immobilizing antigen (e.g., native or variant TSHβ) on a solid support. In such assays, the immobilized antigen “captures” antibody from solution. Alternatively, binding kinetics or binding affinity may be determined using ELISA-based methods, or using biosensor-based technology, such as Biacore surface plasmon resonance technology (Biacore International AB, Uppsala, Sweden). Many such methods are known to those skilled in the art (see, e.g., “Antibody Engineering: A Practical Approach” (McCafferty et al., eds., Oxford University Press, Oxford, United Kingdom, 1996), Goldberg et al., Curr. Opin. Immunol. 5:278-281, 1993, Karlsson et al., J. Immunol. Methods 145:229-240, 1991, Malmqvist, Curr. Opin. Immunol. 5:282-286, 1993, and Hoogenboom, supra).

The binding kinetics or binding affinity of a Fab fragment that selectively binds to native or variant TSHβ may also be determined. Fab fragments do not multimerize. Multimerization may, in certain instances, complicate the measurement of binding kinetics and binding affinity in “solid phase” methods. Thus, Fab fragments that selectively bind to native or variant TSHβ may be suitable for use in certain binding assays in which antigen is immobilized to a solid support, such as, for example, an ELISA-based or Biacore assay. Fab fragments may be generated from an intact antibody that selectively binds to native or variant TSHβ using enzymatic methods, or by expressing nucleic acids encoding Fab fragments in a recombinant expression system.

Alternatively, the binding kinetics or binding affinity of an antibody against native or variant TSHβ can be determined using “solution phase” methods. The measurement of the binding kinetics or the binding affinity of multivalent antibodies and antibodies that multimerize are amenable to solution phase analysis. In such techniques, the kinetics or affinity of binding is measured for an antibody-antigen complex in solution. Such techniques are known to those skilled in the art, including, but not limited to, the “kinetic exclusion assay” (see, e.g., Blake et al., J. Biol. Chem. 271:27677-27685, 1996, and Drake et al., Anal. Biochem. 328:35-43, 2004). Sapidyne Instruments, Incorporated (Boise, Id.), among others, provides instrumentation for performing kinetic exclusion assays. These types of assays may be used to characterize antibodies that can be used to identify TSHβ levels in patents that are thought to be at some risk, or are known to be suffering from TSHβ-related disorders.

Monoclonal antibodies raised, for example, against mouse native or variant TSHβ may be screened for selective binding to human, dog, cat or horse native or variant TSHβ using routine detection methods, such as those described herein. The ability of a monoclonal antibody to selectively bind both mouse and human native or variant TSHβ or those of other mammals (i.e., “cross-reactivity”) indicates the presence of the same epitope in mouse and human native or variant TSHβ or other mammal native or variant TSHβ. In detection methods that use denaturing conditions (e.g., Western blot), cross-reactivity indicates the monoclonal antibody binds to the same “linear” epitope in mouse and human native or variant TSHβ. In detection methods that use non-denaturing conditions, cross-reactivity indicates the monoclonal antibody binds to the same linear epitope or conformational epitope in mouse and human and other mammal native or variant TSHβ.

The epitope to which a monoclonal antibody binds may be identified by any of a number of assays (see, e.g., Morris, Methods Mol. Biol. 66:1-9, 1996). For example, epitope mapping may be achieved by gene fragment expression assays or peptide-based assays. In a gene fragment expression assay, for example, nucleic acids encoding fragments of native or variant TSHβ are expressed in prokaryotic cells and isolated. The ability of a monoclonal antibody to bind those fragments is assessed, e.g., by immunoblotting or immunoprecipitation. Nucleic acids encoding fragments of native or variant TSHβ can be transcribed and translated in vitro in the presence of radioactive amino acids. The radioactively labeled fragments of native or variant TSHβ may then tested for binding to a monoclonal antibody. Fragments of native or variant TSHβ may also be generated by proteolytic fragmentation. An epitope may also be identified using libraries of random peptides displayed on the surface of phage or yeast, or a library of overlapping synthetic peptide fragments of native or variant TSHβ, and testing for binding to a monoclonal antibody. An epitope may also be identified using a competition assay, such as those described below.

Monoclonal antibodies that bind to the same epitope of native or variant TSHβ as a monoclonal antibody of interest may be identified by epitope mapping, as described above, or by routine competition assays (see, e.g., “Antibodies: A Laboratory Manual”, supra, Ch. 14). In an exemplary competition assay, native or variant TSHβ or a fragment thereof, is immobilized onto the wells of a multi-well plate. The monoclonal antibody of interest is labeled with a fluorescent label (e.g., fluorescein isothiocyanate) by standard methods, and then mixtures of the labeled monoclonal antibody of interest and an unlabeled test monoclonal antibody are added to the wells. The fluorescence in each well is quantified to determine the extent to which the unlabeled test monoclonal antibody blocks the binding of the labeled monoclonal antibody of interest. Monoclonal antibodies may be deemed to share an epitope if each blocks the binding of the other by 50% or greater.

Alternatively, to determine if two or more monoclonal antibodies bind the same epitope, epitope binning may be performed (see, e.g., Jia et al., J. Immunol. Methods 288:91-98, 2004), using, for example, Luminex® 100 multiplex technology and the Luminex® 100™ analyzer (Luminex Corporation, Austin, Tex.). Epitope binning typically utilizes an antibody sandwich-type competition assay, in which a “probe” antibody is tested for binding to an antigen bound by a “reference” antibody. If the probe antibody binds to the same epitope as the reference antibody, it will not bind efficiently to the antigen, because that epitope is masked by the reference antibody. Immunoassays based on the above described technologies and devices (both those named and implied) are employed in various embodiments to detect native or variant TSHβ in patents that are thought to be at risk for TSHβ-related disorders.

Antibodies directed against native or variant TSHβ, or conserved variants or peptide fragments thereof, which are discussed above, may also be used to identify and quantify TSHβ from patients with TSHβ-related disorders, as well as in diagnostic and/or prognostic assays, as described herein. Such diagnostic and/or prognostic methods may be used to detect abnormalities in the level of native or variant TSHβ in a patient's body or tissues and may be performed in vivo or in vitro, such as, for example, on biopsy tissue. For example, antibodies directed to epitopes of native or variant TSHβ can be used in vivo to detect the level of TSHβ present in the body. Such antibodies can be labeled, e.g., with a radio-opaque or other appropriate compound, and injected into a subject, in order to visualize cells bearing native or variant TSHβ in the body, using methods such as X-rays, CAT-scans, or MRI.

Alternatively, immunoassays or fusion protein detection assays may be utilized on biopsy and autopsy samples in vitro to permit assessment of the expression pattern of native and variant TSHβ. Such assays may include the use of antibodies directed to epitopes of any of the domains of native or variant TSHβ. For example, in various embodiments antibodies, or fragments thereof, are used to quantitatively or qualitatively detect native or variant TSHβ, conserved variants, or peptide fragments thereof. This may be accomplished, for example, by immunofluorescence techniques employing a fluorescently labeled antibody coupled with ultraviolet microscopic, flow cytometric, or fluorometric detection.

The TSHβ antibodies (or fragments thereof) can be used to determine the level of cells bearing native or variant TSHβ, and can additionally, be employed histologically, for example in immunofluorescence, immunoelectron microscopy, or non-immuno assays, for in situ detection of TSHβ. In situ detection may be accomplished by removing a histological specimen from a patient, and applying thereto a labeled antibody, performing some embodiments of a TSHβ immunoassay. The antibody (or fragment) is preferably applied by overlaying the labeled antibody (or fragment) onto a biological sample. Through the use of such a procedure, it is possible to determine not only the presence of native or variant TSHβ, or conserved variants or peptide fragments, but also its distribution in the examined tissue.

Immunoassays and non-immunoassays for TSHβ will typically comprise incubating a sample, such as a blood or tissue sample, freshly harvested cells, or lysates of cells that have been incubated in cell culture, in the presence of a detectably labeled antibody or antibodies capable of identifying native or variant TSHβ, or conserved variants or peptide fragments thereof, and detecting the bound antibody by any of a number of techniques well-known in the art. The biological sample may be brought in contact with and immobilized onto a solid phase support or carrier such as nitrocellulose, or other solid support that is capable of immobilizing cells, cell particles, or soluble proteins. The support may then be washed with suitable buffers, followed by treatment with the detectably labeled TSHβ antibody or fusion protein. The solid phase support may then be washed with the buffer a second time to remove unbound antibody or fusion protein. The amount of bound label on solid support may then be detected by conventional means.

The terms “solid phase support” or “carrier” are intended to include any support or carrier capable of binding an antigen or an antibody. Well-known supports or carriers include, but are not limited to, glass, polystyrene, polypropylene, polyethylene, polyvinylidene fluoride, dextran, nylon, amylases, natural and modified celluloses, polyacrylamides, gabbros, and magnetite. The nature of the carrier may be either soluble to some extent or insoluble. The support material may have virtually any possible structural configuration, provided that the coupled molecule is capable of binding to an antigen or antibody. Thus, the support configuration may be spherical, as in a bead, or cylindrical, as in the inside surface of a test tube, or the external surface of a rod. Alternatively, the surface may be flat, such as a sheet or test strip. Preferred supports include polystyrene or magnetic beads. Those skilled in the art will know many other suitable carriers for binding antibody or antigen, or will be able to ascertain the same by use of routine experimentation.

The binding activity of a given lot of TSHβ antibody may be determined according to well-known methods. Those skilled in the art will be able to determine operative and optimal assay conditions for each determination by employing routine experimentation.

One of the ways in which a TSHβ antibody may be detectably labeled is by linking the same to an enzyme for use in an enzyme immunoassay (see, e.g., “Immunoassays: A Practical Approach” (Gosling, ed., Oxford University Press, Oxford, United Kingdom, 2000)). The enzyme that is bound to the antibody will react with an appropriate substrate, preferably a chromogenic substrate, in such a manner as to produce a chemical moiety that may be detected, for example, by spectrophotometric, fluorimetric, or visual means. These assays are read and analyzed using chromatometers, spectrophotmeters and fluorometers, respectively. Enzymes that may be used to detectably label the antibody include, but are not limited to, malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, glucose oxidase, asparaginase, beta-galactosidase, ribonuclease, urease, catalase, glucose-6-phosphate dehydrogenase, glucoamylase and acetylcholinesterase. The detection may be accomplished by colorimetric methods that employ a chromogenic substrate for the enzyme. The detection may also be accomplished using methods that employ a fluorogenic substrate in an enzyme-lined fluorescence (ELF) assay. Detection may also be accomplished by visual comparison of the extent of enzymatic reaction of a substrate in comparison with similarly prepared standards.

Additionally, detection may also be accomplished using any of a variety of other immunoassays. For example, by radioactively labeling TSHβ antibodies or antibody fragments, it is possible to detect and quantify TSHβ through the use of a radioimmunoassay (RIA). The radioactive isotope may be detected, for example, by using a gamma or scintillation counter, or by autoradiography. Such antibodies or fragments may also be labeled with a fluorescent compound. When a fluorescently labeled antibody is exposed to light of the proper wavelength, it may be detected due to fluorescence. Exemplary fluorescent labeling compounds include, but are not limited to, fluorescein isothiocyanate, rhodamine, phycoerythrin, phycocyanin, allophycocyanin, o-phthaldehyde, and fluorescamine. Such antibodies may also be detectably labeled using a fluorescence emitting metal, such as ¹⁵²Eu, or others of the lanthanide series. These metals may be attached to an antibody or fragment using such metal chelating groups as diethylenetriaminepentacetic acid (DTPA) or ethylenediaminetetraacetic acid (EDTA).

A TSHβ antibody, or fragment thereof, also may be detectably labeled by coupling it to a chemiluminescent compound. The presence of the chemiluminescent-tagged antibody or fragment is detected by luminescence that arises during the course of a chemical reaction. Examples of useful chemiluminescent labeling compounds include, but are not limited to, luminol, isoluminol, theromatic acridinium ester, imidazole, acridinium salt and oxalate ester. Likewise, a bioluminescent compound may be used to label the TSHβ antibodies, in some cases. Bioluminescence is a type of chemiluminescence found in biological systems, in which a catalytic protein increases the efficiency of the chemiluminescent reaction. The presence of a bioluminescent antibody or fragment is once again detected by luminescence. Exemplary bioluminescent compounds for purposes of labeling include, but are not limited to, luciferin, luciferase and aequorin (green fluorescent protein; see, e.g., U.S. Pat. Nos. 5,491,084, 5,625,048, 5,777,079, 5,795,737, 5,804,387, 5,874,304, 5,968,750, 5,976,796, 6,020,192, 6,027,881, 6,054,321, 6,096,865, 6,146,826, 6,172,188 and 6,265,548).

Free native or variant TSHβ and that bound to the TSH receptor can be identified using various technologies known to those of skill in the art for example, preparative scale immunoprecipitations is used to detect the presence of native or variant TSHβ. Monodispersed magnetic beads are also available as a support material which offers certain advantages over polydisperse agarose beads. Magnetic beads have the ability to bind extremely large protein complexes and the complete lack of an upper size limit for such complexes, as unlike agarose beads which are sponge-like porous particles of variable size, magnetic beads are small, solid and (in the case of monodisperse magnetic beads) spherical and uniform in size. The lower overall binding capacity of magnetic beads for immunoprecipitation make it much easier to match the quantity of antibody needed for diagnostic immunoprecipitations precisely with the total available binding capacity on the beads which results in decreased background and fewer false positives. The increased reaction speed of the immunoprecipitations using magnetic bead technologies results in superior results when the analyte protein is labile due to the reduction in protocol times and sample handling requirements which reduces physical stresses on the samples and reduces the time that the sample is exposed to potentially damaging proteases. Agarose bead-based immunoprecipitations can also be performed more quickly using small spin columns to contain the agarose resin and quickly remove unbound sample or wash solution with a brief centrifugation (Celis, J. E., Lauridsen, J. B., and Basse, B. (1994) Determination of antibody specificity by Western blotting and immunoprecipitation. In: Celis, J. E. (ed.), Cell Biology. A Laboratory Handbook, Academic Press, New York, Vol. 2, pp. 305-313. Mason, D. W., and Williams, A. F. (1986) Kinetics of antibody reactions and the analysis of cell surface antigens. In: Weir, D. M., Herzenberg, L. A., Blackwell, C., and Herzenberg, L. A. (ed.), Handbook of Experimental Immunology, Blackwell, Oxford, vol. 1, chapter 38). Cell bound TSHβ can be identified and using, but not limited to flow cytometry, fluorescence activated cell sorting (FACS™) as well as those methods based on magnetic beads such as Magnetic-activated cell sorting (MACS™) (see for example, Flow Cytometry First Principles by Alice Longobardi Givan (ISBN 0471382248), Practical Flow Cytometry by Howard M. Shapiro (ISBN 0471411256), Flow Cytometry for Biotechnology by Larry A. Sklar (ISBN 0195152344), Handbook of Flow Cytometry Methods by J. Paul Robinson, et al. (ISBN 0471596345), Current Protocols in Cytometry, Wiley-Liss Pub. (ISSN 1934-9297), Flow Cytometry in Clinical Diagnosis, v4, (Carey, McCoy, and Keren, eds), ASCP Press, 2007. (ISBN 0891895485), Ormerod, M. G. (ed.) (2000) Flow cytometry—A practical approach. 3rd edition. Oxford University Press, Oxford, UK. Ormerod, M. G. (1999) Flow Cytometry. 2nd edition. Bios Scientific Publishers, Ltd. Oxford. Flow Cytometry—a Basic Introduction. Michael G. Ormerod, 2008. (ISBN 978-0955981203). Thus, it can be appreciated that a wide variety technologies are currently available to implement the identification of TSHβ and TSHβ bearing cells for the prevention or treatment of TSHβ-related disorders.

Various additional aspects are described in greater detail in the subsections below.

### Examples

### Variant Mouse TSHβ

A novel TSHβ splice variant was identified in hematopoietic cells from mouse bone marrow (BM) using quantitative RT-PCR (qRT-PCR). The mice were 6-8 week old female C57BL/6 mice purchased from Harlan Sprague-Dawley™ (Indianapolis, Ind.). Care and use of mice were in accord with University of Texas Health Science Center at Houston™ institutional animal welfare guidelines. qRT-PCR analysis was done using primers targeted to several regions of mouse TSHβ mRNA using RNA isolated with an RNAeasy Protect Mini Kit™-50. Samples were treated with DNase using an RNase-Free DNase Set-50 (Qiagen™; Valencia, Calif.) according to the manufacturer's protocols. RNA concentrations were determined at A₂₆₀. Primer sets were purchased from IDT Technologies™ (Coralville; IA) or Superarray Bioscience™ Corp. (Frederick, Md.). qRT-PCR was performed on 100 ng total RNA using an iScript One-Step RT-PCR™ kit with SYBR Green™ (Bio-Rad™; Hercules, Calif.). A blank sample with RNase-free water was used for primer controls. Amplification was done in 96-well thin-wall plates sealed with optical quality film in a Mini-Opticon™ (Bio-Rad™) with a program of 10 min at 50° C. for cDNA synthesis, 5 min at 95° C. for reverse transcriptase inactivation, followed by 45 cycles of 95° C. for 10 s and 55° C. for 30 s for data collection. A melt curve was performed using a protocol of 1 min at 95° C., 1 min at 55° C., increasing the temperature in 0.5° C. increments for 80 cycles of 10 s each. Real-time PCR data were quantified using the 2^{-ΔΔCt} method of Livak and Schmittgen, 2001 (Analysis of relative gene expression data using real-time quantitative PCR and the 2(-Delta Delta C(T)) Method. Methods 2001; 25(4):402-8), samples were normalized to respective GAPDH values using a Gene Expression Macro Version 1.1 program (Bio-Rad™). The full-length mRNA sequence is shown in FIG. 1A and SEQ ID NO: 1), which indicates the positions of the five mouse TSHβ exons (designated E1-E5), with the translated portion beginning with the ATG (bolded) at the second nucleotide of exon 4 and extending to the TAA stop codon in exon 5.

A set of primers designated ‘470’ was used for PCR amplification with pituitary and bone marrow RNAs (SEQ ID NOS: 13 and 14). Those primer sequences, which span the known TSHβ coding region, were targeted to a region in exon 3 (470-5′; SEQ ID NO: 13) and exon 5 (470-3′; SEQ ID NO: 14). Also see SEQ ID NO: 1 and FIG. 1A. Using the 470 primer set, Applicants consistently observed a marked difference (26,987 fold greater) in the amount of amplified product for pituitary vs. bone marrow RNA (FIG. 1B). That pattern also held true using five additional upstream primers targeted to regions in exon 4 (designated UP1-UP5; SEQ ID NOS: 15-19, respectively) (FIGS. 1A and 1B) with a downstream primer targeted to exon 5 (designated 98-3′ or 98 reverses; SEQ ID NO: 21). Conversely, when qRT-PCR analysis was done using two primer sets targeted to exon 5 (98-5′ to 98-3′, and Superarray; FIG. 1A; SEQ ID NOS: 20-21), the fold difference in gene expression between pituitary vs. BM was 648 and 439, respectively (FIG. 1B). This represented a statistically-significant (p<0.01) 62.8-fold reduction (34,019 vs. 543) in the relative gene expression of the ratio of pituitary/BM TSHβ expression using upstream primer sequences targeted to exons 3 or 4 compared to primers targeted to exon 5 (FIG. 1B).

The inventors hypothesized that the qRT-PCR differences between BM and pituitary RNAs as a function of the primer target location were due to alternative splicing of the TSHβ gene at or near the junction of exons 4 and 5. In order to obtain a sequence of bone marrow TSHβ mRNA from that region, 5′ RACE analysis was done using a highly-purified preparation of BM RNA. RACE technology insured that only full-length, non-truncated mRNAs were used. 5′ RACE was done using a GeneRacer™ Kit (Invitrogen®; Carlsbad, Calif.). Briefly, highly pure RNA isolated from BM cells was dephosphorylated with calf intestinal phosphatase to insure that only full-length non-truncated mRNA was used. RNA was treated with tobacco acid pyrophosphatase to remove the 5′ cap structure from intact full-length mRNA. A 5′ RACE Oligo provided with the kit (SEQ ID NO: 22) was ligated to the 5′ end of the mRNA. The ligated mRNA was reverse transcribed using SuperScript III™ reverse transcriptase to create a RACE-ready first strand cDNA. The cDNA was amplified using Platinum Pfx™ DNA polymerase with the 5′ RACE Oligo primer and a TSH gene specific primer (GSP; SEQ ID NO: 23). The RACE PCR products were purified using an S.N.A.P. column provided with the kit. PCR products were cloned into a pCR4BLUNT-TOPO vector using a TOPO™ cloning kit (INVITROGEN). Chemically competent E. coli were transformed with 4 μl of the 5′ RACE PCR product. Transformed cells were then selected based on kanamycin resistance. The clones were grown overnight in LB broth in the presence of kanamycin. Plasmid DNA was purified using a QIAGEN QIAprep™ Spin Miniprep Kit (QIAGEN, Inc; Valencia, Calif.). Sequencing was done by Seqwright, Inc. (Houston, Tex.) using M13 primers.

A sequence, which was consistently obtained in multiple 5′ RACE cDNA clones, is shown in FIG. 1C and SEQ ID NO: 9. The underlined nucleotide regions are the 5′ RACE oligo and the 3′ TSHβ GSP (FIG. 1C). A gene blast search revealed complete homology to a portion of the mouse TSHβ gene as shown in FIG. 1D. A striking finding from these experiments was that all of the 5′ RACE sequences obtained from BM RNA included a portion of intron 4 that was contiguous with exon 5. A potential ATG (methionine) start codon is followed by a sequence that codes for 9 amino acids (MLRSLFFPQ) that are in-frame with TSHβ exon 5 beginning at nucleotide 186 (FIG. 1C). The analysis was performed by using a program for identifying an open reading frame (available at the National Library of Medicine at NIH at www.ncbi/nlm/nih.gov). ATG comprises an open reading frame with a Kozak sequence consisting of the ATC prior to the ATG triplet. Without being bound by theory, these data point to a modified splicing mechanism for BM TSHβ, which explains the low levels in PCR product from BM RNA using upstream primer sequences targeted to exons 3 or 4 vs. the abundance of product using primers targeted to exon 5.

Expression of Native TSHβ and Variant TSHβ in the Mouse

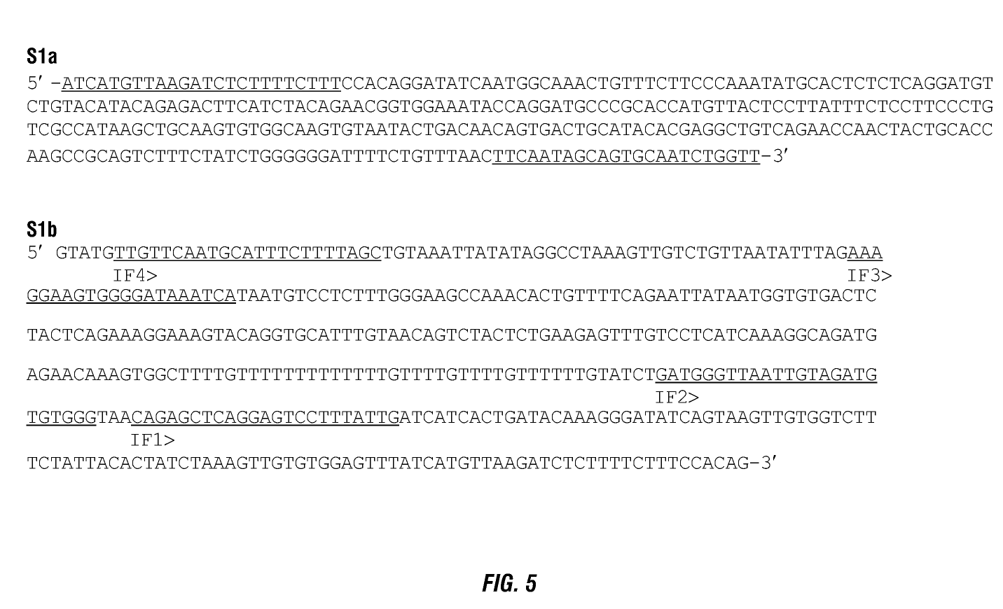

PCR was used to determine the level of expression of both native and variant TSHβ. Relative to the expression of native TSHβ, the TSHβ splice variant was expressed at low level in pituitary cells but at high levels in BM and thyroid cells. Conventional and realtime PCR analyses were done to determine whether the novel TSHβ splice variant was expressed in pituitary, BM, and/or thyroid tissues. For this, a new primer set designated ‘novel primers’ was used (FIG. 5—S1a; SEQ ID NOS: 24-25). The new primer set consisted of a 24 nucleotide upstream primer targeted to intron 4 (SEQ ID NO: 24), and a downstream primer targeted to a sequence located just after the TAA stop codon of exon 5 (SEQ ID NO: 25). If present, the novel TSHβ transcript would be amplified using these. FIG. 2A indicates the relative gene expression levels using the 470 primer set and the TSHβ novel primer set. By conventional PCR, transcript levels using the 470 primers were detectable only in pituitary RNA (FIG. 2A, top panel), whereas the novel TSHβ transcript was present in all three tissues—the pituitary, the BM, and the thyroid. These findings were confirmed by qRT-PCR (FIG. 2B), which indicated an overwhelming preference for the native TSH using the 470 primer set with pituitary vs. BM or thyroid RNA. In contrast, the amount of levels of qRT-PCR amplification were more similar in those three tissues (FIG. 2B), implying that there is a preferential use of the novel TSHβ splice variant in the BM and thyroid.

Because the novel TSHβ product was generated using an upstream primer targeted to a sequence of intron 4, experiments were done to rule out that amplification had occurred from genomic DNA. Three experiments confirmed that amplification was not due to genomic DNA. First, if genomic DNA were present, amplification with the 470 primer sets would yield larger PCR products due to the presence of introns 3 and 4. As seen in FIG. 2A (top panel), all three samples were devoid of PCR products larger than the anticipated 470 nucleotide size. Second, when BM RNA was tested in a one-step PCR reaction in the absence or in the presence of reverse transcriptase using the novel upstream primer, a PCR product was obtained only in the presence of reverse transcriptase and not in the absence of reverse transcriptase (FIG. 2C). Third, using four upstream primer sequences targeted to regions of intron 4 (FIG. 5 S1b; SEQ ID NOS: 26-29), all yielded products of the correct size from genomic DNA, but only the two primers for regions at the beginning of the RACE sequence yielded products from BM RNA (FIG. 2D). The latter not only confirmed the lack of genomic DNA in RNA preparations, but it also validated the 5′ RACE findings as accurately defining the beginning of the BM TSHβ splice variant.

Recombinant TSHβ Proteins:

To obtain recombinant proteins, native TSHβ and splice variant TSHβ DNAs were subcloned into pcDNA3.1/V5-His-TOPO™ vectors. Plasmid DNA was obtained using standard methods. CHO cells grown in serum-free CHO-CD media™ (Sigma-Aldrich™; St. Louis, Mo.) were transfected with native or novel plasmid DNA using an Amaxa electroporator (Amaxa Biosystems™; Gaithersburg, Md.). Cells were selected for stable transfectants by continuous culture in 1.2 mg/ml neomycin. 10⁷ cells were used to purify His-tagged recombinant proteins using a NI-NTA Fast Start™ Kit (Qiagen™). Estimate of protein concentration was determined using a Coomassie Plus-200 Protein assay™ (Pierce™; Rockford, Ill.). Recombinant proteins were stored at −80° C. in the presence of 0.5% bovine serum albumin for stabilization.

Immunoprecipitation was done using an anti-TSHβ antibody directed to a portion of the molecule that is shared by both native and novel TSHβ to confirm that transfected CHO cells produced TSHβ of the correct molecular size. 2×10⁶ non-transfected CHO cells, CHO cells transfected with native TSHβ, and CHO cells transfected with splice variant TSHβ were lysed on ice for 15 min in buffer consisting of 50 mM Tris-HCL (pH 7.4), 150 mM NaCl, 1 mM EGTA, 2 μg/ml aprotinin 1 μg/ml leupeptin, and 1 mM phenylmethyl sulfonyl fluoride (Sigma-Aldrich™, all reagents). Lysates were clarified by high speed centrifugation in a microfuge for 5 min. Supernatants were collected and pre-cleared by end-over-end mixing overnight with 50 μl of protein G plus agarose (Santa Cruz Biotechnologies™; Santa Cruz, Calif.). Protein G was removed from lysates by three successive centrifugation. Lysates were reacted by end-over-end mixing for 1 hr at 4° C. with 25 μl of anti-TSHβ antibody (N-19) (Santa Cruz Biotechnologies™) adsorbed to protein G. Protein G immune complexes were washed with lysis buffer and suspended in 30 μl of Laemmli sample buffer (Bio-Rad™; Hercules, Calif.) containing 5% β-mercaptoethanol, boiled for 5 min, and electrophoresed though a pre-cast 12% polyacrylamide gel (Bio-Rad™). Gels were fixed and exposed to silver staining using the reagents and methods of M. Barton Frank (http://omrf.ouhsc.edu/˜frank/SILVER.html).

The enzyme-linked immunoassay (EIA) used was similar to a procedure developed and published using an anti-mouse TSHβ antibody (Zhou Q, Wang H C, Klein J R. Characterization of novel anti-mouse thyrotropin monoclonal antibodies. Hybrid Hybridomics 2002; 21(1):75-9).

Statistical Analyses: Determination of statistical significance was done by ANOVA or by a t-test for two samples with unequal variance.

Variant TSHβ Protein is an Actively Secreted Protein:

The physiochemical characteristics of the novel TSHβ polypeptide predicted from the nucleotide sequence is shown in FIG. 3 and SEQ ID NO: 4. This consists of a 9 amino acid leader sequence followed by an eighty-four amino acid polypeptide of the mature protein molecule coded for by exon 5 up to the TSHβ stop codon (FIG. 3A). The difference between the novel TSHβ polypeptide (FIG. 3B, underlined residues) and the native TSHβ molecule is the lack of amino acids coded for by exon 4. The secondary structure of the novel TSHβ splice variant is shown in FIG. 3C. Without being bound by theory, the high hydrophobic moment index (grey line; >3.0) and the high transmembrane helix preference for the first 9 amino acids indicates that the protein may favor a transmembrane location for a likely signal peptide function.

Importantly, cell-free supernatants from CHO cells transfected with native and splice variant TSHβ constructs had high levels of TSHβ as detected by reactivity with an anti-mouse TSHβ-specific monoclonal antibody (FIG. 3D). Supernatants from control CHO cells transfected with a LacZ construct were non-reactive with the anti-TSHβ antibody. Since CHO cells were not transfected with the TSHβ gene, it is assumed that the observed activity is due to TSHβ alone. These findings collectively indicate that the novel TSHβ product is actively secreted from cells.

Cell lysates from non-transfected CHO cells were non-reactive by immunoprecipitation (FIG. 3E). Immunoprecipitation of cell lysates from CHO cells transfected with the native TSHβ construct produced a 17 kDa product. An 8 kDa product was precipitated from lysates of CHO cells transfected with the novel TSHβ construct (FIG. 3E).

Variant TSHβ is Biologically Active:

Recombinant native and splice variant proteins were also used to evaluate their ability to induce a cAMP response from mouse AM cells and rat FRTL-5 cells. FRTL-5 cells were obtained from American Type Culture Collection™ (Manassas, Va.). The moue alveolar macrophage cells line, AMJ2-C8 (American Type Culture Collection™), hereafter referred to as AM, was a gift from Dr. Chinnaswamy Jagannath, Department of Pathology, The University of Texas Health Science Center at Houston™. FRTL-5 cells were grown in DMEM supplemented with 10% FBS, 2 mM L-glutamine, 10 μg/ml insulin, 10 nM hydrocortisone, 5 μg/ml transferrin, 10 ng/ml gly-his-lys-acetate, 10 ng/ml somatostatin, and 10 mU/ml bovine TSH (Sigma-Aldrich™). AM cells were grown in defined serum-free hybridoma medium (Gibco-Invitrogen™; Carlsbad, Calif.) supplemented with 2 mM L-glutamine. 5-7 days prior to stimulation with native and splice variant TSHβ, media lacking bovine TSH was used for FRTL-5. FRTL-5 cells were grown to 80% confluency in 24 well Corning tissue culture plates (Fisher Scientific™; Pittsburgh, Pa.). AM cells were grown to a density of 1−2×10⁶/ml. As described by others, FRTL-5 cells were washed twice with HBSS containing 0.4% BSA, 220 mM sucrose, and 1 mM isobutylmethylxanthine (Sigma-Aldrich™). AM cells were washed and seeded at a density of 1×10⁶ cell/ml in 1.5 ml eppendorf tubes. Log₁₀M dilutions of recombinant native TSHβ, splice variant TSHβ, forskolin, or media for control cultures was added for 3 hr at 37° C. in a humidified 5% CO₂ incubator. Cell free supernatants were collected and assayed for cAMP activity using a commercial assay kit (R&D Systems™; Minneapolis, Minn.).

Mouse AM cells were confirmed to express the TSH receptor by RT-PCR (data not shown). Culture of AM cells with recombinant native and splice variant TSHβ induced a cAMP responses with native TSHβ having peak activity at a concentration of 10⁻¹° M, and splice variant TSHβ having peak activity at a concentration of 10⁻⁶M (FIG. 4A). Differences in those responses as a function of TSH concentration may reflect differences in receptor binding activities. Future studies are planned to examine this possibility.

Using FRTL-5 cells, both native and the novel TSHβ splice variant induced dose-dependent cAMP responses (FIG. 4B). Moreover, the optimal molar concentrations for these responses (10⁻¹⁰-10⁻¹²) were typical of TSH concentration used by others to induce cAMP responses in vitro. The cAMP response induced by recombinant TSHβ proteins, although low, were generally in line with some previous reports of cAMP responses from FRTL-5 cells (Chico Galdo V, Massart C, Jin L, et al. Acrylamide, an in vivo thyroid carcinogenic agent, induces DNA damage in rat thyroid cell lines and primary cultures. Mol Cell Endocrinol 2006; 257-258:6-14). A lower cAMP response from FRTL-5 cells also may be due to poor binding of mouse TSH to rat FRTL-5 cells, an interpretation that is supported by the fact that bovine TSH generated a stronger cAMP response from FRTL-5 cells (8.83 pmol/ml and 7.75 pmol/ml cAMP following stimulation with 10⁻⁷ M and 10⁻⁹ M bovine TSH, respectively). Collectively, these findings confirm that the polypeptide made from the TSHβ splice variant is biologically active with regard to its ability to induce a cAMP signal.

TSHβ Splice Variant Upregulated in the Thyroid During Systemic Virus Infection:

To determine if immune challenge by reovirus would result in changes in intrathyroidal levels of native and/or splice variant TSHβ, C57BL/6 mice were infected i.p. with 10^{7.5} pfu T3D reovirus. Use of reovirus was based on studies that demonstrated altered thyroid function following reovirus infection (Neufeld D S, Platzer M, Davies T F. Reovirus induction of MHC class II antigen in rat thyroid cells. Endocrinology 1989; 124(1):543-5; Srinivasappa J, Garzelli C, Onodera T, Ray U, Notkins A L. Virus-induced thyroiditis. Endocrinology 1988; 122(2):563-6). Reovirus serotype 3 Dearing strain (T3D reovirus) was purchased from the American Type Culture Collection (Manassas, Va.). Virus stocks were grown as previously described in (Montufar-Solis D, Garza T, Teng B B, Klein J R. Upregulation of ICOS on CD43⁺ CD4⁺ murine small intestinal intraepithelial lymphocytes during acute reovirus infection. Biochem Biophys Res Commun 2006; 342(3):782-90). Mice were infected i.p. with 10^{7.5} pfu T3D reovirus or with PBS to serve as non-infected control animals. Mice were euthanized after 2 days and thyroid tissues were recovered and used for RNA extraction for qRT-PCR analysis with native and splice variant primers.

RNA was extracted from the thyroid tissues isolated 48 hrs post infection, and qRT-PCR was done using the 470 and the novel TSHβ primer sets (previously described). It was determined that systemic virus infection did not alter the level of native TSHβ gene expression in the thyroid relative that of non-infected mice. However, there was a statistically-significant increase in gene expression of the TSHβ splice variant in the thyroid of virus-infected mice (FIG. 4B). These findings suggest that the intrathyroidal host response is linked to the TSHβ splice variant but not the native form of TSHβ.

Variant Human TSHβ

Having identified a novel splice variant isoform of the TSHβ subunit in mice that was preferentially produced by hematopoietic cells and by cells in the thyroid, and which is upregulated in the thyroid during systemic virus infection (shown in example above), the inventors sought to identify a TSHβ splice variant isoform in humans.

Identification and Tissue Expression of Human TSHβ Splice Variant Isoform:

Two primer sets were used for analysis of human TSHβ gene expression. One set, designated native TSHβ, was designed to amplify the complete human TSHβ open reading frame. The native TSHβ primer set consisted of an upstream primer targeted to a region in exon 2 prior to the TSHβ transcriptional start site (FWD TSHβ Human Native PCR Primer: 5′-AGCATGACTGCTCTCTTTCT-3′; SEQ ID NO: 30), and a downstream primer targeted to a region in exon 3 that began one nucleotide after the stop codon (RVR TSHβ Human Native and Novel PCR Primer: 5′-AACCAAATTGCAAATTATATCACTA-3′; SEQ ID NO: 31).

The second primer set (designated novel TSHβ) consisted of an upstream primer targeted to a region at the end of intron 2 (FWD TSHβ Human Novel PCR Primer: 5′-ATTATGCTCTCTTTTCTGTTCTTT-3′; SEQ ID NO: 32) with the same downstream primer used for native TSHβ (SEQ ID NO: 31).

PCR amplification was done using RNAs from human pituitary, thyroid, PBL, and BM.

Highly pure RNA from human pituitary, bone marrow, peripheral blood leukocytes (PBL), and thyroid tissues were purchased from BioChain Institute (Hayward, Calif.) from tissues that were obtained with informed consent. The following additional primers were also used:

FWD TSHα Human PCR Primer: 5′-ATGGATTACTACAGAAAATATGC-3′ (SEQ ID NO: 33); and

RVR TSHα Human PCR Primer: 5′-AGATTTGTGA TAATAACAAG TACT-3′ (SEQ ID NO: 34).

Primers were constructed and purchased from IDT Technologies (Coralville; IA). cDNA were made from RNA using an iScript cDNA Synthesis Kit (Bio-Rad; Hercules, Calif.) with a program of 5 minutes at 25° C., 30 minutes at 42° C., and 5 minutes at 85° C. An iScript kit with SYBR Green (Bio-Rad) was used for qRT-PCR using 20 ng cDNA A blank sample with RNase-free water was used for primer controls. Amplification was done in 96-well thin-wall plates sealed with optical quality film in a Mini-Opticon (Bio-Rad) using a program of 45 cycles of 95° C. for 10 s and 55° C. for 30 s for data collection. A melt curve was performed using a protocol of 1 min at 95° C., 1 min at 55° C., and increasing the temperature in 0.5° C. increments for 80 cycles of 10 s each. Gene expression values were normalized to 18s values for respective tissue RNAs according to a previously-disclosed method (Livak and Schmittgen, 2001) using a Gene Expression Macro Version 1.1 program (Bio-Rad). PCR-amplified products were analyzed by electrophoresis through a 1.2% agarose gel run at 60 V for 90 min followed by 10 min stain with 0.3 mg/ml ethidium bromide and a 10 min water de-stain.

RT-PCR yielded both native and novel TSHβ PCR products from pituitary RNA, but only novel TSHβ from thyroid and PBL RNAs (FIG. 6). Neither form of TSHβ was amplified from BM RNA (FIG. 6A). RT-PCR resulted in a product for TSHα from pituitary, thyroid, and PBL, but not BM (FIG. 6B). 18s gene expression was expressed at equivalent levels in all four samples (FIG. 6C).

To measure the differences in native vs. novel TSHβ gene expression, qRT-PCR was conducted. Gene expression values were normalized to 18s values for respective tissues RNAs using the method of Livak and Schmittgen (Livak and Schmittgen, 2001). Although both native and novel TSHβ forms were expressed in the pituitary, there was a 111-fold preference for native over novel TSHβ in the pituitary. This pattern was reversed in the thyroid and PBL where there was a 4,374-fold preference, and a 955-fold preference of novel over native TSHβ gene expression in the thyroid and in PBL, respectively.

qRT-PCR analysis was done to determine if the TSHα gene was expressed in tissues that expressed the TSHβ splice variant. The pattern of gene expression observed for the TSHβ splice variant also was present for TSHα as seen by high level of expression in the pituitary, modest level of expression in the thyroid, low but detectable expression level in PBL, and undetectable levels of TSHα in BM.

The identified variant TSHβ isoform in humans consisted of a twenty-seven nucleotide portion of intron 4 that is contiguous with the coding region of exon 5 of mouse TSHβ, resulting in a polypeptide that comprises 71.2% of the native TSHβ molecule.

Sequence of the Human TSHβ Splice Variant Isoform:

The human novel TSHβ sequence was subcloned into the pCR2.1 TOPO plasmid using the TOPO TA Cloning kit (Invitrogen; Carlsbad, Calif.). Briefly, the human novel TSHβ sequence was Taq-amplified from thyroid tissue using the following touchdown PCR program: 4 minutes at 95° C.; 10 cycles of 95° C. for 30 seconds, 65° C. and −1°/cycle for 60 seconds, and 72° C. for 90 seconds; 20 cycles of 95° C. for 30 seconds, 50° C. for 60 seconds, and 72° C. for 90 seconds; and a final 7 minute elongation step at 72° C. 2 μl of the resulting PCR product was included in the TOPO reaction and incubated at room temperature for 30 minutes prior to transformation of the TOP10 bacteria. Ampicillin-resistant clones were selected and plasmid DNA was isolated using the QIAprep™ Spin Miniprep Kit (Qiagen, Valencia, Calif.). Positive clones were identified by restriction digest with EcoRI. Sequences were obtained from SegWright™ (Houston, Tex.) using the M13R primer. Sequence analysis was performed using FinchTV™ v1.3.1s software and an NCBI BLAST search.

Sequence analysis of the novel TSHβ PCR product revealed complete homology to human TSHβ (GenBank accession no. NM 000549) (FIG. 8, SEQ ID NOS: 5 and 10), including the twenty-seven nucleotides in intron 2 that precede exon 3. Moreover, seven of the nine amino acids coded for by human TSHβ intron nucleotides were identical to mouse TSHβ within that region. Hence, there was a high degree of organizational similarity between the human and the mouse TSHβ splice variant.

A comparison of the nucleotide sequence of the human and mouse TSHβ splice variant is shown in FIG. 9A. Within the twenty-seven nucleotide region of the putative leader sequence (FIG. 9A, underlined nucleotides), eight nucleotides differed between the two species. However, this resulted in only two amino acid substitutions, as shown in FIG. 9B at amino acid positions three and four of the leader sequence. Because those substitutions consisted of amino acids that were primarily hydrophobic or uncharged polar, a potential transmembrane function of the human leader sequence remains likely, as was previously predicted for the mouse TSHβ splice variant molecule.

Without further elaboration, it is believed that one skilled in the art can, using the description herein, utilize the present invention to its fullest extent. The embodiments described herein are to be construed as illustrative and not as constraining the remainder of the disclosure in any way whatsoever. While the preferred embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

Applications of Variant TSHβ

The experiments below illustrate without limitation that various embodiments of the present disclosure have numerous applications.

Suppression of TSHβ Splice Variant Expression Using siRNA

TSHβ pSilencer 4.1-CMV Puro Construction:

The 27 nucleotide sequence of the mouse TSHβ splice variant located in intron 4 (first 27 nucleotides, FIG. 12A) was submitted to the siRNA Target Finder and siRNA Converter design program (Insert Design Tool for the pSilencer™ Vectors programs; Ambion, Austin, Tex.) to obtain candidate structures to serve as a TSHβ splice variant shRNA hairpin construct. This search identified the underlined nucleotide sequence (FIG. 12A) as a candidate for shRNA targeting for use in RNA-inhibition of the TSHβ splice variant isoform. Oligonucleotides for this were purchased from IDT, Inc. (Coralville, Iowa) and annealed to yield the oligonucleotide duplex shown in FIG. 12B (SEQ ID NOS: 11-12). Duplexes were cloned into the pSilencer 4.1-CMV puro expression vector (Ambion), which has a CMV promoter, a poly A signal region, and puromycin and ampicillin cassettes. Sequencing was performed to verify the identity of the final TSHβ pSilencer 4.1-CMV puro plasmid.

Generation of Stable TSHβ pSilencer AM Cell Line and Quantification of TSHβ Expression:

To determine the effects of TSHβ splice variant-specific shRNA on gene expression, a stable mammalian cell line was generated using the construct shown in FIG. 12B. The mouse AM cell line, an alveolar macrophage cell line that expresses high levels of the TSHβ splice variant, was transfected with the TSHβ pSilencer 4.1-CMV puro plasmid using an Amaxa Nucleofector (Lonza; Basel, Switzerland). Forty-eight hours post-transfection, puromycin was added to the cultures to generate a stable TSHβ pSilencer AM cell line.

Mock-transfected AM and stable TSHβ pSilencer AM cells were collected and processed for quantitative real-time PCR (qRT-PCR) to measure expression of TSHβ splice variant transcript levels at 48 hours post-transfection, and after 4 weeks of selection with puromycin. RNA was isolated using an RNeasy Protect Minikit (Qiagen; Valencia, Calif.) according to the manufacturer's instructions. For analysis of TSHβ isoform gene expression, SYBR Green qRT-PCR was performed using the iScript One-Step RT-PCR Kit with SYBR Green (BioRad; Hercules, Calif.) according to the manufacturer's instructions. Gene expression levels were determined by normalizing to GAPDH. As shown in FIG. 10, there was an early (48 hr) and persistent (4 wk) suppression of the TSHβ splice variant with no adverse effect on expression of the 18s housekeeping gene in the AM cells.

Suppression of Circulating T4 Levels by TSHβ Splice Variant Recombinant Proteins

Experimental Protocol Used for Determination of T4 Suppressive Effects by Recombinant Mouse TSHβ

To determine if the TSHβ splice variant isoform would alter the levels of circulating thyroid hormone, groups of C57BL/6 mice were injected intraperitoneally once daily for three days with either PBS (5 mice), or with 3 ug of recombinant TSHβ splice variant protein made in E. coli in Applicants' laboratory (4 mice). Twenty-four hours after the last injection, mice were euthanized, blood was collected, and total T4 levels were measured in the sera. Exposure of mice to the TSHβ splice variant protein resulted in a statistically-significant (p<0.001) 36.6% reduction in circulating T4 (FIG. 11). These findings demonstrate that the TSHβ splice variant protein can suppress thyroid hormone production in vivo.

As a summary, primers used in some of the Examples of the present disclosure are listed in Table 1 below.

TABLE 1
Primers used in some embodiments
of the present invention.

| Primer designation | Sequence |
|---|---|
| 470 forward | 5′-AAGAGCTCGGGTTGTTCAAA-3′ |
| (SEQ ID NO: 13) | |
| 470 reverse | 5′-CACATTTAACCAGATTGCACTG-3′ |
| (SEQ ID NO: 14) | |
| UP1 forward | 5′-TCTCCGTGCT TTTTGCTCTT-3′ |
| (SEQ ID NO: 15) | |
| UP2 forward | 5′-GCAAGCAGCATCCTTTTGTA-3′ |
| (SEQ ID NO: 16) | |
| UP3 forward | 5′-CGTGGATAGGAGAGAGTGTGC-3′ |
| (SEQ ID NO: 17) | |
| UP4 forward | 5′-TCAACACCACCATCTGTGCT-3′ |
| (SEQ ID NO: 18) | |
| UP5 forward | 5′-TGCTGGGTATTGTATGACACG-3′ |
| (SEQ ID NO: 19) | |
| 98 forward | 5′-CCGCACCATGTTACTCCTTA-3′ |
| (SEQ ID NO: 20) | |
| 98 reverse | 5′-ACAGCCTCGTGTATGCAGTC-3′ |
| (SEQ ID NO: 21) | |
| 5′ RACE oligo | 5′-CGACTGGAGCACGAGGACACTG |
| (forward) | AC-3′ |
| (SEQ ID NO: 22) | |
| TSHβ GSP (reverse) | 5′-TGCGGCTTGGTGCAGTAGTTGG |
| (SEQ ID NO: 23) | TTCTG-3′ |
| Novel TSHβ forward | 5′-ATCATGTTAAGATCTCTTTTCT |
| (SEQ ID NO: 24) | TT-3′ |
| Novel TSHβ reverse | 5′-AACCAGATTGCACTGCTATTGA |
| (SEQ ID NO: 25) | A-3′ |
| Intron primer 4 | 5′-TTGTTCAATGCATTTCTTTTAG |
| forward | C-3′ |
| (SEQ ID NO: 26) | |
| Intron primer 3 | 5′-GAAAGGAAGTGGGGATAAATCA-3′ |
| forward | |
| (SEQ ID NO: 27) | |
| Intron primer 2 | 5′-GATGGGTTAATTGTAGATGTGTG |
| forward | G-3′ |
| (SEQ ID NO: 28) | |
| Intron primer 1 | 5′-CAGAGCTCAGGAGTCCTTTATT |
| forward | G-3′ |
| (SEQ ID NO: 29) | |
| FWD TSHβ human native | 5′-AGCATGACTGCTCTCTTTCT-3′ |
| (SEQ ID NO: 30) | |
| RVR TSHβ human | 5′-AACCAAATTGCAAATTATATCA |
| native/novel | CTA-3′ |
| (SEQ ID NO: 31) | |
| FWD TSHβ human novel | 5′-ATTATGCTCTCTTTTCTGT |
| (SEQ ID NO: 32) | |
| FWD human TSHα | 5′-ATGGATTACTACAGAAAATATG |
| (SEQ ID NO: 33) | C-3′ |
| RVR human TSHα | 5′-AGATTTGTGA TAATAACAAGT |
| (SEQ ID NO: 34) | ACT-3′ |
| Superarray TSHβ | Cat. No. PPM30787A |
| (forward and reverse) | |
| Superarray GAPDH | Cat. No. PPM02946E |
| (forward and reverse) | |

Without further elaboration, it is believed that one skilled in the art can, using the description herein, utilize the present invention to its fullest extent. The embodiments described herein are to be construed as illustrative and not as constraining the remainder of the disclosure in any way whatsoever. While some embodiments may involve particular mammals, the present invention encompasses other mammals, including experimental animals, companion animals, farm animals, primates and humans. While the preferred embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. An isolated nucleic acid molecule that encodes the amino acid sequence of SEQ ID NOS: 4 or 8.

2. The isolated nucleic acid molecule of claim 1, wherein said nucleic acid molecule comprises the nucleotide sequence of SEQ ID NOS: 3 or 7.

3. An expression vector comprising the isolated nucleic acid molecule of claim 1.

4. The expression vector of claim 3, wherein said expression vector is selected from the group consisting of a plasmid, cosmid, bacteriophage, and viral expression vector.

5. The expression vector of claim 4, wherein said viral expression vector is selected from the group consisting of a baculovirus, cauliflower mosaic virus, CaMV, tobacco mosaic virus, and TMV.

6. A host cell comprising the expression vector of claim 3.

7. The host cell of claim 6, wherein said host cell is a eukaryotic cell.

8. The host cell of claim 7, wherein said eukaryotic cell is selected from the group consisting of COS cells, CHO cells, fibroblasts, VERO cells, BHK cells, HeLa cells, MDCK cells, 293 cells, 3T3 cells, and WI38 cells.

9. The host cell of claim 6, wherein said host cell is a prokaryotic cell.

10. The host cell of claim 9, wherein said prokaryotic cell is E. coli.

11. A method of producing a polypeptide, wherein said method comprises culturing the host cell of claim 6 under conditions that permit the expression of the expression vector of claim 3.

12. A substantially isolated polypeptide comprising the amino acid sequence of SEQ ID NOS: 4 or 8.

13. A substantially isolated antibody that binds TSH or TSH-β, wherein said antibody has immunospecificity for an epitope comprising the first 9 amino acids of SEQ ID NOS: 4 or 8.

14. The antibody of claim 13, wherein said antibody is a native antibody selected from the group consisting of IgM, IgD, IgG, IgA, IgE, and IgG.

15. The antibody of claim 13, wherein said antibody is an antibody fragment selected from the group consisting of Fab, Fab′, F(ab′)2, Fv, scFv, Fd, diabodies, and other antibody fragments that retain at least a portion of a variable region of an intact antibody.

16. A method of treating a subject with a TSHβ-related disorder, wherein said method comprises the administration of a TSH protein to said subject, and wherein said TSH protein comprises a variant TSHβ chain comprising the amino acid sequence of SEQ ID NOS: 4 or 8.

17. The method of claim 16, wherein said treatment comprises an intravenous administration of said TSH protein to said subject.

18. The method of claim 16, wherein said treatment comprises an oral administration of said TSH protein to said subject.

19. The method of claim 16, wherein said TSH-β-related disorder comprises at least one of adenoma, thyroid hormone resistance, hypopituitarism, hyperthyroidism, Graves' disease, congenital hypothyroidism (cretinism), hypothyroidism, Hashimoto's thyroiditis, autoimmune thyroid diseases, Graves' Ophthalmopathy, thyroid nodules, Pendred's Syndrome, post-traumatic stress disorder, Lyme disease, osteoporosis, obesity, infertility, autoimmune and inflammatory disorders, inflammation associated with single-organ or multi-organ failure or sepsis, and inflammation associated with chronic active hepatitis, alcoholic liver disease, or non-alcoholic fatty liver disease.

20. A method of treating a subject with a TSH-β-related disorder, wherein said method comprises the administration of an antagonist to said subject, and wherein said antagonist has specificity for an epitope comprising the first 9 amino acids of SEQ ID NOS: 4 or 8.

21. The method of claim 20, wherein said antagonist is the substantially isolated antibody of claim 13.
